SEQUENCE LISTING (27 sequences)

SEQ ID NO: 1 - DNA, 43 bp, synthetic construct
  misc_feature 1..43: note = oligonucleotide ON1
  source 1..43: mol_type = other DNA
agcaaatgtctagattatccctaacatttaaatgcaatgaaaa

SEQ ID NO: 2 - DNA, 10 bp, synthetic construct
  misc_feature 1..10: note = oligonucleotide ON2
  source 1..10: mol_type = other DNA
tccctttttt

SEQ ID NO: 3 - DNA, 28 bp, synthetic construct
  misc_feature 1..28: note = oligonucleotide ON3
  source 1..28: mol_type = other DNA
tctagattatcccttttttttttttttt

SEQ ID NO: 4 - DNA, 48 bp, synthetic construct
  misc_feature 1..48: note = oligonucleotide ON4
  source 1..48: mol_type = other DNA
gaagcaaatgtctagattatcccttttttttttttttttttttttttt

SEQ ID NO: 5 - DNA, 67 bp, synthetic construct
  misc_feature 1..67: note = oligonucleotide ON5
  source 1..67: mol_type = other DNA
gaagcaaatgtctagattatccctttttttttttttttttttttttttttttttgtttaaacattta

SEQ ID NO: 6 - DNA, 51 bp, synthetic construct
  misc_feature 1..51: note = oligonucleotide ON6
  source 1..51: mol_type = other DNA
agggggggagggagaggggtaccccctatccttagtcaccaccggccccct

SEQ ID NO: 7 - DNA, 38 bp, synthetic construct
  source 1..38: mol_type = other DNA
gggagaggggtacccccttttttttttttttttttttt

SEQ ID NO: 8 - DNA, 48 bp, synthetic construct
  source 1..48: mol_type = other DNA
ctttgactcccatggtggcgtacccccttttttttttttttttttttt

SEQ ID NO: 9 - DNA, 31 bp, synthetic construct
  source 1..31: mol_type = other DNA
tgttttgttttacccctttggtttttttttt

SEQ ID NO: 10 - DNA, 38 bp, synthetic construct; identical to SEQ ID NO: 7
  source 1..38: mol_type = other DNA

SEQ ID NO: 11 - DNA, 43 bp, synthetic construct
  source 1..43: mol_type = other DNA
ggagggagaggggtaccccctaaacatttaaatgcaatgaaaa

SEQ ID NO: 12 - DNA, 1332 bp, synthetic construct
  misc_feature 1..1332: note = plasmid PL1
  source 1..1332: mol_type = other DNA
gccgaattatattatttttgccaaataatttttaacaaaagctctgaagtcttcttcatttaaattcttagatgatacttcatctggaaaattgtcccaattagtagcatcacgctgtgtaatacgactcactatagggggtacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcaggtgggagggtacgttcgtgataaacaaac

SEQ ID NO: 13 - DNA, 1179 bp, synthetic construct
  misc_feature 1..1179: note = plasmid PL2
  source 1..1179: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgtaatacgactcactatagggggtacgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcaggtgggagggtacgttcgtgataaacaaac

SEQ ID NO: 14 - DNA, 1137 bp, synthetic construct
  misc_feature 1..1137: note = plasmid PL3
  source 1..1137: mol_type = other DNA
ctgcagccgaattatattatttttgccaaataatttttaacaaaagctctgaagtcttcttcatttaaattcttagatgatacttcatctggaaaattgtcccaattagtagcatgccacgctgtgtaatacgactcactatagggataatctagacatttgcttctgacacaactgtgttcactagcaacctcaaacagacaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgc

SEQ ID NO: 15 - DNA, 1667 bp, synthetic construct
  misc_feature 1..1667: note = plasmid PL4
  source 1..1667: mol_type = other DNA
cctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 16 - DNA, 1111 bp, synthetic construct
  misc_feature 1..1111: note = plasmid PL5
  source 1..1111: mol_type = other DNA
cctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtaatgagtcctggactgaaacggactcatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 17 - DNA, 1287 bp, synthetic construct
  misc_feature 1..1287: note = plasmid PL6
  source 1..1287: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtaggcgcgccagtccttctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaagcgactcggacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 18 - DNA, 1667 bp, synthetic construct
  misc_feature 1..1667: note = plasmid PL7
  source 1..1667: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaagagacggggtacgttcgtgatacggccgcttcgagcaga

SEQ ID NO: 19 - DNA, 1092 bp, synthetic construct
  source 1..1092: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgtaatacgactcactatagggggtacgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 20 - DNA, 1678 bp, synthetic construct
  source 1..1678: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactataggggtaaaacaaaacacacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatggtgtctaagggggaggagctgttcaccggcgtggtgcccatcctggtggagctggacggcgacgtgaacggccacaagttcagcgtgagcggggagggcgagggcgacgccacctacggcaagctgaccctgaagttcatctgcacaacggggaagctgcccgtgccctggcccaccctggtgaccaccctgacctacggcgtgcagtgcttcagccggtaccccgaccacatgaagcagcacgacttcttcaagagcgccatgcccgaggggtacgtgcaggagcggaccatcttcttcaaggacgacgggaactacaagacccgcgccgaggtgaagttcgagggggacaccctggtgaaccgcatcgagctgaagggcatcgacttcaaggaggacggcaacatcctggggcacaagctggagtacaactacaacagccacaacgtgtacatcatggccgacaagcagaagaacggcatcaaggtgaacttcaagatccggcacaacatcgaggacggcagcgtgcagctggccgaccactaccagcagaacacccccatcggcgacggccccgtgctcctgcccgacaaccactacctgtccacccagagcgccctgagcaaggacccaaacgagaagcgggaccacatggtcctgctggagttcgtgaccgccgccggcatcaccctgggcatggacgagctgtacaagcgctcccgcgacatcagccacggcttccctcccgccgtggccgcccaggacgacggcaccctgcccatgagctgcgcccaggagagcggcatggaccggcacccggccgcctgcgccagcgcccggatcaacgtgtgatagggatccaacaaaaaaaacaaaaaaaaaaccaaagagacgcggccgcttcgagcagacatgataagatacatt

SEQ ID NO: 21 - DNA, 1793 bp, synthetic construct
  source 1..1793: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagcggccgcttcgagcagacatgataagatacattgatgatc

SEQ ID NO: 22-23 - skipped (2 entries)

SEQ ID NO: 24 - DNA, 16 bp, synthetic construct
  source 1..16: mol_type = other DNA
aaaaaaaaagcaggtg

SEQ ID NO: 25-26 - skipped (2 entries)

SEQ ID NO: 27 - DNA, 21 bp, synthetic construct
  source 1..21: mol_type = other DNA
ccttcagctcgatgcggttca

(19) United States
(12) Patent Application Publication
(10) Pub. No.: US 2026/0317365 A1
(43) Pub. Date: Oct. 15, 2026

(54) CIRCULAR RNA ANALOGS COMPRISING 7-METHYLGUANOSINE CAP, PREPARATION, AND APPLICATION THEREOF

(71) Applicant: Siec Badawcza Lukasiewicz PORT Polski Osrodek Rozwoju Technologii, Wroclaw (PL)

(72) Inventors: Adam Mamot, Warszawa (PL); Malgorzata Wasinska-Kalwa, Warszawa (PL); Katarzyna Frankowska, Warszawa (PL); Karol Czubak, Skarzysko Koscielne (PL); Marcin Warminski, Warszawa (PL); Joanna Kowalska, Warszawa (PL); Jacek Jemielity, Warszawa (PL); Dominika Nowis, Warszawa (PL); Jakub Golab, Warszawa (PL)

(21) Appl. No.: 19/485,434

(22) PCT Filed: May 20, 2024
(86) PCT No.: PCT/EP2024/063844

§ 371 Date: Nov. 18, 2025

(30) Foreign Application Priority Data
May 18, 2023 (EP) 23461592.0

(51) Int. Cl. C07H 21/02; C07H 1/00

(52) U.S. Cl. CPC C07H 21/02; C07H 1/00

(57) ABSTRACT

The presented invention relates to a novel class of nucleic acids, which have circular topology and contain at least one unnatural internucleotide linkage containing a 7-methylguanosine cap analog.

### SUMMARY OF THE INVENTION

The presented invention relates to a novel class of nucleic acids, which have circular topology and contain at least one unnatural internucleotide linkage containing a 7-methylguanosine cap analog. The main objects of the disclosed invention are analogs of circular RNA (circRNA) that are obtained by the synthesis of 5′-modified precursors (pre-circRNAs) and their subsequent circularization via a chemical reaction. The circularization occurs due to the presence of two chemically reactive functional groups, one at RNA 5′ end and another at the 3′ end. The functional group at the 5′ end is incorporated co-transcriptionally, whereas the reactive group at the 3′ end is incorporated post-transcriptionally. The invention encompasses RNAs of different lengths (from 2 nt to 20000 nt), including messenger RNAs (RNAs encoding a protein). The circRNAs encoding a protein according to this invention undergo translation with an efficiency higher than chemically unmodified circRNAs of the same sequence. One of the embodiments of the invention discloses circRNA analogs that are obtained from pre-circRNAs containing a functionalized 7-methylguanosine cap analog at their 5′ end. Such circRNAs containing caps are more efficiently translated than circRNAs of the same sequence without the cap. Methods of preparation of circRNA analogs, methods of preparation of corresponding pre-circRNAs, methods of preparation of a protein or peptide, and application of the disclosed circRNA analogs in the context of gene therapies and vaccinations are also disclosed.

### FIELD OF THE INVENTION

The presented invention relates to RNA molecules, methods of producing circular RNA molecules, methods of producing a protein or peptide in vitro, in cells, or living organisms, and methods of treating or preventing a disease.

### BACKGROUND OF THE INVENTION

Despite its inherent fragility, RNA is one of the most potent substances in the context of therapeutic gene delivery.¹ The potency of therapeutic messenger RNA (mRNA) stems from both advanced methods of delivery and chemical modification of the mRNA itself. Both in serum and in the cytosol, mRNA is primarily targeted by exonucleases (e.g. decapping complexes, deadenylases, exosomes).² Circularization is one of the most protective modifications of mRNA, as it prevents exonucleolytic digestion.³

Currently known methods of RNA circularization are based on chemical reactions^{4, 5}, enzymatic reactions,⁶⁻⁹ and reactions catalyzed by catalytic sequences embedded in circRNA precursors¹⁰⁻¹³ (pre-circRNA).

The reported methods of circularization that are based on chemical reactions rely on the use of short RNA sequences, chemically synthesised via phosphoramidite method, such as 3′ phosphorylated RNA or 3′-amino RNA, which undergo condensation induced by cyanogen bromide (BrCN) or 1-ethyl-3-(3-dimethylaminopropyl) carbodiimide (EDC).^{4, 5} The phosphoramidite solid phase synthesis of nucleic acids is a robust method of production of modified and unmodified oligonucleotides, however it is not practical for the production of long (>100 nt) RNA sequences. In consequence, circRNA obtained with these methods encode relatively short sequences and has limited use in the context of RNAs encoding a protein or peptide, which are usually significantly longer.

Moreover, RNA circularization can be carried out with ligases, especially T4 RNA ligase I and T4 RNA ligase II.^{6, 7, 14} Both enzymes require 5′ monophosphorylated precursors, which are first adenylated and subsequently ligated with the 3′ OH of the ribose at the 3′ end of a different or the same RNA strand.¹⁵ Both enzymes are capable of intramolecular ligation (circularization) of RNA (if reaction occurs with the 3′ end of the same RNA strand); however, they have been found to be strongly dependent on target RNA length and sequence.^{11, 12, 14, 16}, This is detrimental, as it difficult to predict or improve performance of such enzymes.

The most recognised methods of RNA circularization involve catalytic nucleic acids.^{8-12, 17} Wesselhoeft et al. have circularized RNA in vitro by using the permuted intron-exon (PIE) system and optimized self-splicing intronic sequences.^{11, 12} The PIE system consists of fused partial exons flanked by half-intron sequences.^{10, 17} During the self-splicing reaction, two transesterification reactions occur. First, 3′ hydroxyl group of a guanosine nucleotide engages 5′ splice site. Second, the freed hydroxyl group at the end of the intermediate engages the 3′ splice site, which results in circularization.

Litke et al. have shown the Twister-optimized RNA durable overexpression (Tornado) system for RNA circularization.⁸ Tornado-expressed transcripts contain an RNA of interest flanked by Twister ribozymes that spontaneously undergo autocatalytic cleavage. This leaves 5′-hydroxyl and 2′,3′-cyclic phosphate termini, which are then ligated by the RNA ligase RtcB.

The so-far-reported methods based on catalytic nucleic acid activity require sequence design and development, therefore cannot be used on pre-circRNA of any given sequence. Furthermore, pre-circRNA's ability to undergo circularization can be hindered by the presence of chemical modifications, such as natural and synthetic nucleosides and nucleobases.¹²

One of the biggest flaws of circRNA in the context of gene delivery and gene therapies is low protein expression. The main pipelines of eukaryotic translational machinery are based on cap-dependent mechanisms.^{18, 19} None of the discovered or synthesised circular RNA (circRNA) contain elements such as 7-methylguanosine cap, or any kind of 5′ nucleoside analog.^{20, 21} Due to circular and “cap-less” structure, the translation of currently known circRNA occurs in an IRES (internal ribosome entry site)-dependent manner.^{5, 7, 9, 11, 12, 19, 22, 23} In eukaryotic cells, cap-dependent translation is much more effective than IRES-dependent translation.¹⁹ On the other hand, the stability of circRNA is higher, in consequence, circRNAs exhibit significantly longer half-lives and the protein expression from these mRNAs lasts longer.^{12, 14}

WO/2021/113777 discloses circular RNAs and transfer vehicles, along with related compositions and methods of treatment. Pharmaceutical compositions comprising such circular RNAs as well as precursor RNAs and materials useful in producing the precursor or circular RNAs have been also disclosed in cited document.

WO/2019/236673 describes a vector for making circular RNA, allowing the production of a circular RNA that is translatable or biologically active inside eukaryotic cells.

EP4008336 discloses a recombinant nucleic acid molecule of the transcriptional circular RNA, recombinant expression vector, pre-circularized RNA, circular RNA, recombinant host cell, pharmaceutical composition and protein preparing method. The transcription product of the recombinant nucleic acid molecule is a circular RNA which contains a specific IRES element.

US20150079630 discloses cyclic RNA and a method for production thereof which comprises the use of T4 DNA ligase.

None of the previously reported methods are suitable for incorporating a 7-methylguanosine cap structure within protein-coding circRNA.

### SUMMARY

The present disclosure provides novel analogs of circRNAs that can be prepared from pre-circRNAs via a chemical reaction (chemical circularization of RNA), regardless of the circRNA sequence, length or size. Due to chemical nature of the circularization process, it enables incorporation of 7-methylguanosine cap within the structure of circRNA. Unexpectedly, the presence of the cap structure within a cricRNA analog molecule increases its translational activity, in comparison to chemically unmodified circRNAs of the same sequence. Therefore, present disclosure gives unprecedented access to circRNAs that undergo translation via cap-dependent mechanism.

The present disclosure provides a circRNA analog prepared by in vitro transcription reaction followed by a subsequent chemical circularization reaction, wherein said circularization is obtained in the absence of protein enzymes or catalytic nucleic acid sequences.

The present disclosure provides a circRNA analog that contains either a 7-methylguanosine cap analog structure or both a 7-methylguanosine cap analog structure and an internal ribosomal entry site sequence.

The present disclosure provides a circRNA analog according to formula 1:

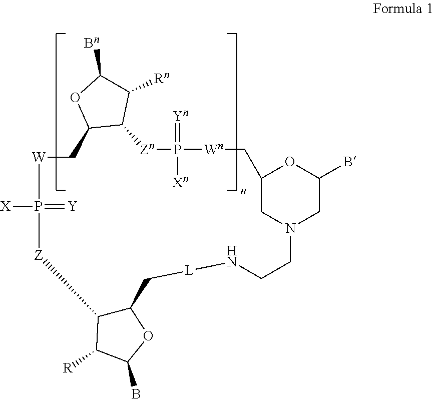

wherein:
n is any integer with a value from 1 to 20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker, wherein said linker L comprises of a natural, modified or unnatural 7-methylguanosine cap structure.

In some embodiments, the linker L is a structure according to Formula 2, Formula 3 or Formula 4,

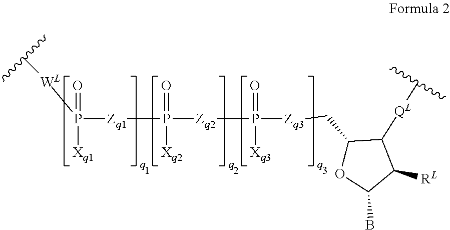

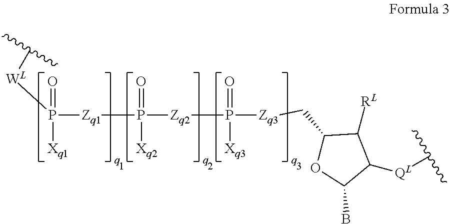

wherein:
B is 7-methylguanine, natural or unnatural 7-methylguanine derivative or analog,
each of q₁ through q₃ is independently 0, 1 or 2, wherein q₁+q₂+q₃ is equal 3 or 4;
X_{q1}, X_{q2}, X_{q3} is independently O, BH₃, or S;
Z_{q1} and Z_{q2} is independently O, NH, CH₂, C (halogen)₂ or CH (halogen);
Z_{q3} is O, S, NH, CH₂, C (halogen)₂ or CH (halogen);
W^{L} is a single chemical bond, O, NH, S, CH₂ or a linear or branched linker;
Q′ is a single chemical bond, CH₂, or a linear or branched linker;
R^{L} is H, OH, O-Me, O-alkyl, O-[(substituted)alkyl].

In some embodiments, the linker L is a structure according to formula 4

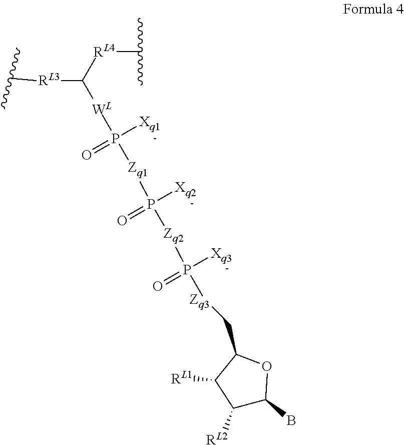

wherein:
B is 7-methylguanine, natural or unnatural 7-methylguanine derivative or analog,
each of q₁ through q₃ is independently 0, 1 or 2, wherein q₁+q₂+q₃ is equal 3 or 4;
X_{q1}, X_{q2}, X_{q3} is independently O, BH₃, or S;
Z_{q1} and Z_{q2} is independently O, NH, CH₂, C (halogen)₂ or CH (halogen);
Z_{q3} is O, S, NH, CH₂, C (halogen)₂ or CH (halogen);
W^{L} is a single chemical bond, O, NH, S, CH₂ or a linear or branched linker;
R^{L1} and R^{L2} are independently H, OH, O-Me, O-alkyl, O-[(substituted)alkyl], wherein at least one of RLI and R^{L2} is not OH;
R^{L3} and R^{L4} are independently single chemical bonds, CH₂ or a linear or branched linker.

In some embodiments, the linker L comprises of a detectable label.

In some embodiments, the linker L is a structure selected among of following formulas:

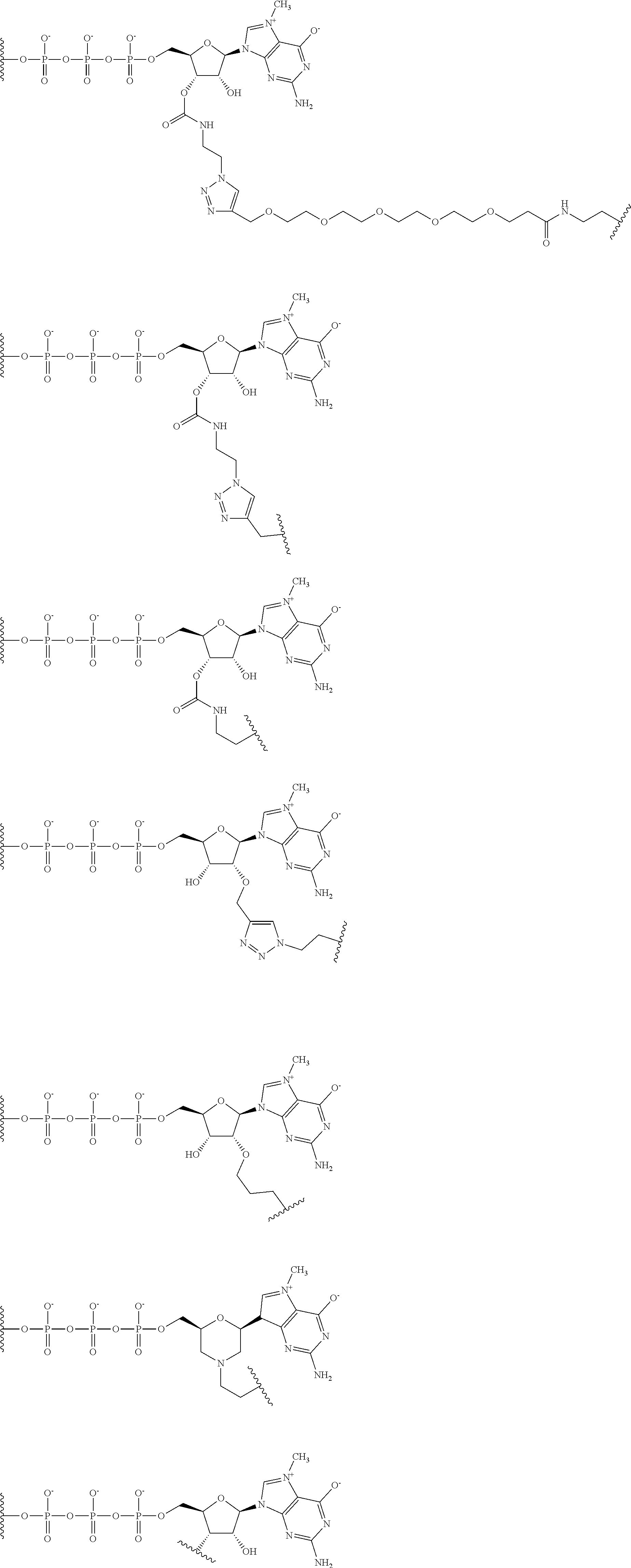

In some embodiments, the linker L is a structure selected among of following formulas:

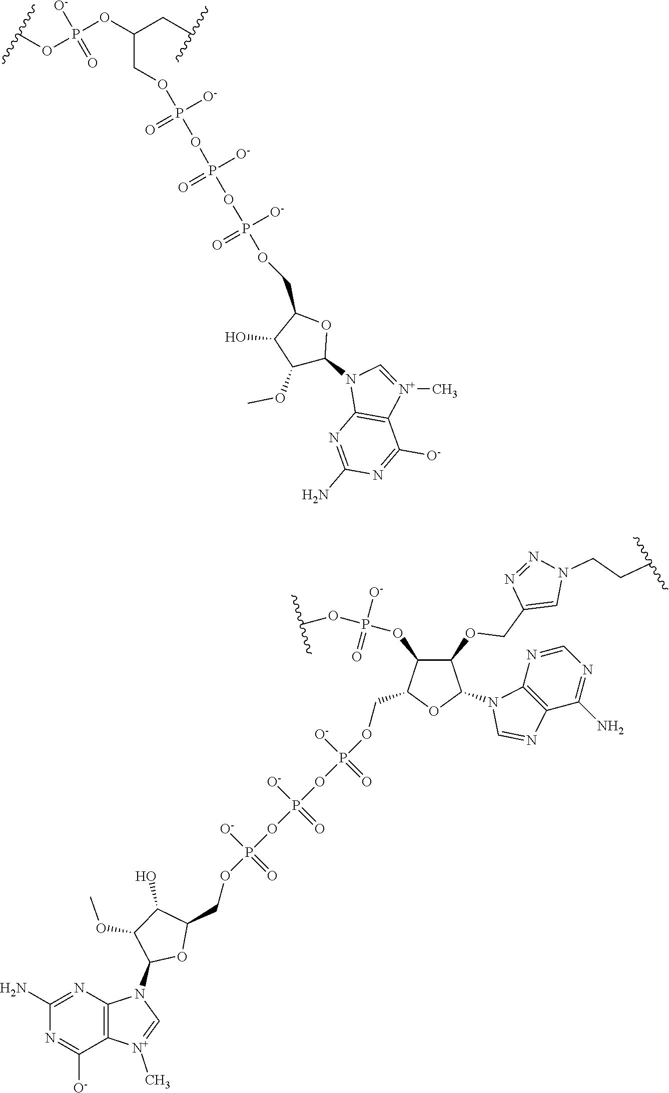 [SMILES: *CC(COP(=O)([O-])OP(=O)([O-])OP(=O)([O-])OC[C@H]1O[C@@H](n2c[n+](C)c3c([O-])nc(N)nc32)[C@H](OC)[C@@H]1O)OP(=O)([O-])O* ; *CCn1cc(CO[C@@H]2[C@H](OP(=O)([O-])O*)[C@@H](COP(=O)([O-])OP(=O)([O-])OP(=O)([O-])OC[C@H]3O[C@@H](n4c[n+](C)c5c([O-])nc(N)nc54)[C@H](OC)[C@@H]3O)O[C@H]2n2cnc3c(N)ncnc32)nn1]

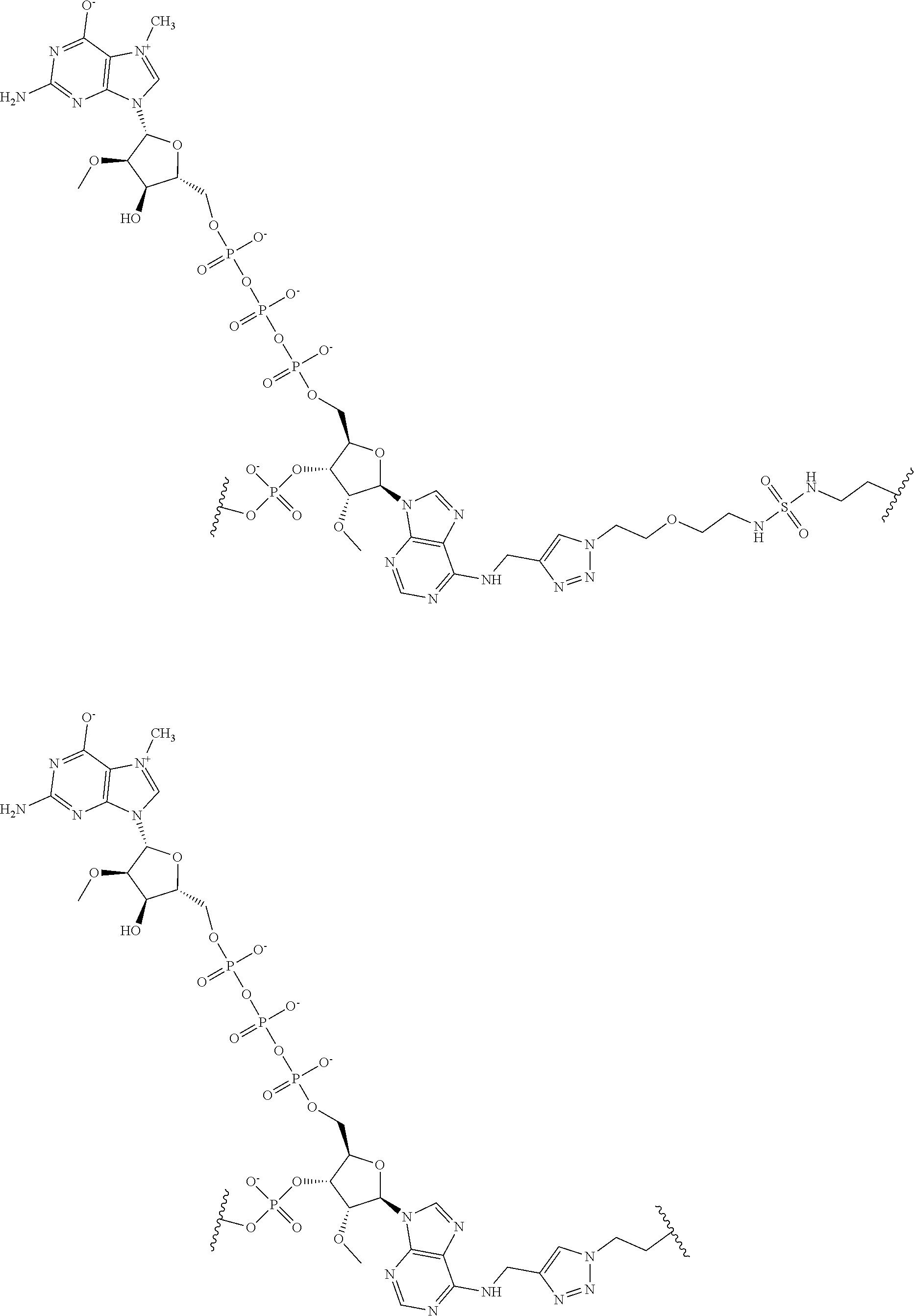 [SMILES: *CCNS(=O)(=O)NCCOCCn1cc(CNc2ncnc3c2ncn3[C@@H]2O[C@H](COP(=O)([O-])OP(=O)([O-])OP(=O)([O-])OC[C@H]3O[C@@H](n4c[n+](C)c5c([O-])nc(N)nc54)[C@H](OC)[C@@H]3O)[C@@H](OP(=O)([O-])O*)[C@H]2OC)nn1 ; *CCn1cc(CNc2ncnc3c2ncn3[C@@H]2O[C@H](COP(=O)([O-])OP(=O)([O-])OP(=O)([O-])OC[C@H]3O[C@@H](n4c[n+](C)c5c([O-])nc(N)nc54)[C@H](OC)[C@@H]3O)[C@@H](OP(=O)([O-])O*)[C@H]2OC)nn1]

The present disclosure provides a method of synthesis of circRNA analogs described above, characterized by that a circRNA analog precursor (formula 7)

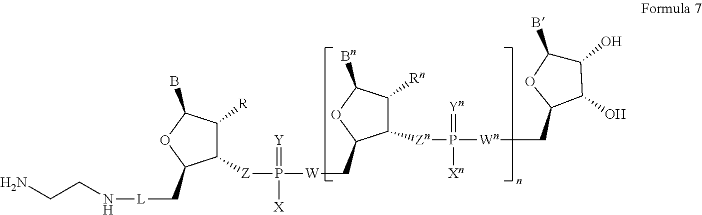

wherein:
n=1-20 000;
X and each of X¹ through Xⁿ is independently OH, BH₃, NH₂, or SH;
Y and each of Y¹ through Yⁿ is independently O or S;
Z and each of Z¹ through Zⁿ is independently O, S or NH;
W and each of W¹ through Wⁿ is independently O, S or NH;
R and each of R^{L} through Rⁿ is independently H, OH, alkyl, O-alkyl or halogen;
B, B′ and each of B¹ through Bⁿ is independently a natural, modified or unnatural nucleoside base;
L is a linear or branched linker; preferably the linker L is a structure selected among of formulas which have been presented above;
is first oxidized during step (i) of the method, yielding a dialdehyde derivative (formula 8),

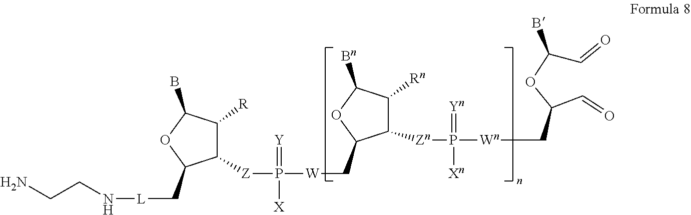

wherein:
n=1-20 000;
X and each of X¹ through Xⁿ is independently OH, BH₃, NH₂, or SH;
Y and each of Y¹ through Yⁿ is independently O or S;
Z and each of Z¹ through Zⁿ is independently O, S or NH;
W and each of W¹ through Wⁿ is independently O, S or NH;
R and each of R^{L} through Rⁿ is independently H, OH, alkyl, O-alkyl or halogen;
B, B′ and each of B¹ through Bⁿ is independently a natural, modified or unnatural nucleoside base;
L is a linear or branched linker;
which is next incubated in reductive environment during step (ii) of the method, yielding the desired circRNA analog.

In some embodiments, step (i) and step (ii) are carried out in one reactor. In some embodiments, step (i) of the method is carried out in the presence of NaIO₄, preferably at a concentration below 10 mM, at a temperature below 60° C. In some embodiments, step (ii) of the method is carried out in a buffered solution, preferably at pH 5-8, in presence of NaBH₃CN below 100 mM. In some embodiments, step (i) or step (ii) of the method are carried out in presence of a natural, modified, or unnatural nucleic acid oligomer that is complementary to a RNA precursor sequence. In some embodiments, the circRNA analog is isolated from the reaction mixture by a known method of RNA isolation, such as precipitation in alcohol, size exclusion chromatography, gel electrophoresis, or high-performance liquid chromatography (HPLC).

The present disclosure provides a use of a circRNA analog described above in protein production in an in vitro setup.

The present disclosure provides a circRNA analog described above for use in a therapeutic or diagnostic method, in particular for protein production in an in vivo setup.

The present disclosure provides a pharmaceutical composition comprising of a circRNA analog described above.

### Definitions

Unless defined otherwise, all terms used herein have the same meaning as are commonly understood by one of skill in the art to which this invention belongs.

The terms “nucleic acid” and “nucleic acid polymer” refer to a polymeric substance composed of ribo or deoxyribo derivatives and of naturally occurring or synthetic nucleoside monomers, including noncanonical linkages and any degree of polymerization. These phrases include, but are not limited to, natural or synthetic RNA and DNA, as well as nucleic acids composed of phosphorothioate, boranophosphate, selenophosphate, and phosphonate moieties, morpholino nucleic acids (MNA), locked nucleic acid (LNA), peptide nucleic acid (PNA), and threose nucleic acid (TNA).

The terms “modification”, “modifying group”, and “modified” in the context of nucleic acids refer to any chemical moiety that may be attached to a nucleic acid molecule. This includes, but is not limited to, attachment of the modifying group to the sugar, nucleobase, internucleotide phosphate, or 5′-5′-oligophosphate of a nucleic acid molecule.

The term “internucleotide linkage” refers to a covalent or non-covalent bond or bonds that connect two monomers of a nucleic acid polymer.

The terms “nucleoside base”, “nucleobase” and “nitrogenous base” refer to a part of nucleic acid molecules. The referred nucleobases may be involved in base pairing, base stacking, and other inter- and intramolecular interactions. The referred nucleobases include, but are not limited to natural, modified, unnatural or synthetic purine (such as adenine, guanine, hypoxanthine, xanthine, N7-methylguanosine, N6-methyladenosine, N2-methyladenosine) and pyrimidine (such as cytosine, thymidine, uracil, dihydrouracil, 5-methylcytosine, 5-hydroxymethylcytosine, pseudouridine, methylpseudouridine, nicotinamide) nucleobases, as well as 2-aminopurine, 2,6-diaminopurine, flavin, C8-substituted adenine, C8-substituted guanine, C7-substituted 7-deazaadenine, C7-substituted 7-deazaguanine, C5-substituted uridine, C5-substituted cytosine, di- and tricyclic nucleobases.

The terms “nucleic acid sequence”, “nucleotide sequence”, “nucleoside sequence”, “nucleobase sequence” and “sequence” refer to the order and composition of a series of nucleobases within a nucleic acid molecule.

The terms “nucleobase pair” and “base pair” refer to a fundamental unit of double-stranded nucleic acids consisting of two nucleobases bound to each other by hydrogen bonds. The term “base pairing” refers to specific hydrogen bonding between nucleobases, that can be canonical (Watson-Crick base paring) or non-canonical (e.g. wobble base paring, Hoogsten base paring).

The terms “hybridization” and “hybridize” refer to interaction of two or more nucleic acid strands. It is characterized by that, that under appropriate conditions distinct nucleic acid strands maintain a sequence-dependent structure (e.g. a double helix, a triple helix, an I-motif, a G-quadruplex).

The term “complementary” refers to relation between sequences of two or more strands of a nucleic acid or nucleic acids. The complementary sequences or strands are characterised by that they undergo hybridization and form a sequence-dependent structure. The complementarity can be full or partial.

The terms “linear” and “circular” in context of nucleic acids refer to the topology of a nucleic acid molecule. Linear nucleic acids are linear polymers with no linkage between 5′ and 3′ ends. Circular nucleic acids are polymers with natural or modified linkage between 5′ and 3′ ends.

The terms “circularization” and “cyclization” refer to a process of transformation of a linear nucleic acid polymer to a circular nucleic acid polymer.

The terms “circRNA precursor” and “pre-circRNA” refer to a linear nucleic acid polymer that can undergo circularization leading to a circular nucleic acid polymer.

The term “linker” in context of circular nucleic acids refers to any natural or synthetic combination of chemical moieties that covalently join 5′ and 3′ ends of the polymer. The term “linker” includes, but is not limited to any linear or branched combination of alkyl, alkenyl, alkynyl, ether, ester, amide, amine, carbamate, triazol, phosphoester, amidophosphate, or phosphonate moieties. In particular it can be selected among of following structures:

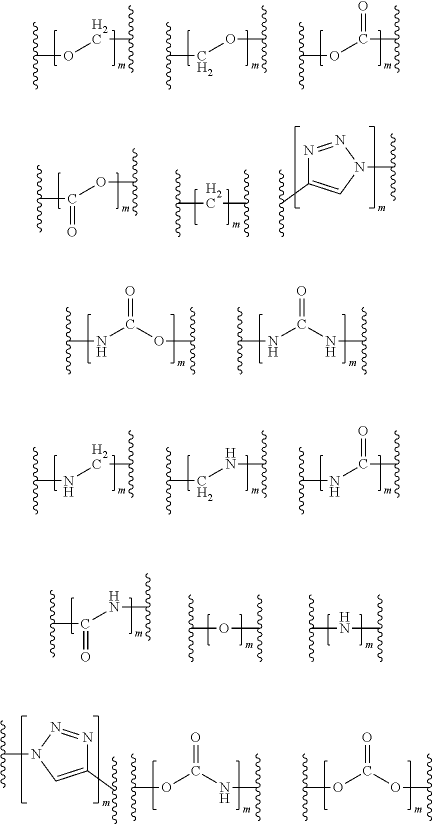

wherein m is a natural number from 1 to 100.

The term “cap analog” refers to any compound that contains natural or modified 5′-5′-oligophosphate linking a natural or modified nucleosides, nucleotides, or nucleic acids. The term “Cap analog” refers to canonical, as well as non-canonical cap structure and includes, but is not limited to Cap0, Cap1, Cap2, TMG-cap, NAD-cap, and FAD-cap, as well as cap structures modified within oligophosphate bridge, ribose, or nucleobases. In particular, it can be selected among of structures described in any document cited in subject description, esspecially: WO/2009/149253, WO/2008/157688, WO/2011/015347, WO/2022/191725, WO/2018/015845, WO/2014/093627, WO/2021/162566, WO/2021/162567 and WO/2017/130151.

The term “natural, modified or unnatural 7-methylguanosine cap structure” refers to any compound that contains 7-methylguanosine or its natural or modified derivative. The term includes, but is not limited to cap analogs such as m⁷GTP, m⁷GpppG, m⁷GpppA, Cap0, Cap1, and Cap2.

The terms “internal ribosomal entry site” and “IRES” refer to RNA sequences that are characterised by their ability facilitate cap-independent initiation of translation by interaction or binding cellular proteins involved in formation of ribosomes and translation. The term “IRES sequence” includes, but is not limited to Encephalomyocarditis virus (EMCV) IRES, Coxsackievirus B³ (CVB3) IRES, respiratory syncytial virus (RSV) m⁶A methylation sites, viral IRES sequences, human IRES sequences, m⁶A methylation sites, sequences containing m⁶A modification, eIF4G aptamers, and artificial IRES sequences.^{11, 12, 22, 24}

The terms “transcription” or “transcription reaction” refer to methods for enzymatic synthesis of RNA that is complementary to a DNA template. Transcription can occur in cells, in cellular systems or in artificial setups (as in in vitro transcription reaction). Transcription is carried out in presence of a DNA-dependent RNA polymerase, ribonucleoside triphosphates (NTPs), and a DNA template. During the reaction, the sequence of the DNA template is copied and amplified in form of RNA molecules. Optionally, the transcription can be carried out in the presence of additional reagents, such as transcription initiators, transcription terminators, or modified nucleoside triphosphates.

The terms “label” and “detectable label” refer to any compound, particle, or any combination of compounds and/or particles, that may be directly or indirectly detected. The label can be associated with a target molecule by covalent and/or non-covalent interactions. The label can be a radioisotope, NMR label, spin label, a chromophore, a fluorophore, a hapten. The preferred label includes but is not limited to ²H, ¹³C, ¹⁵C, ³²P, ¹⁹F, cyanines, fluoresceines, rhodamines, coumarines, perylenes, naphthalimides, nitro dyes, azo dyes, BODIPY, biotin, desthiobiotin, folic acid and N-acetylgalactosamine.

The terms “periodate oxidation”, “cis-diol oxidation” and “ribose oxidation” refer to a process of cleavage of carbon-carbon bonds of vicinal cis-diols present in the structure of a natural or synthetic nucleic acids leading to a dialdehyde derivative of a nucleic acid.

The terms “reductive amination” and “amination” in the context of nucleic acid modification or circularization refer to amination reaction involving a dialdehyde derivative of a nucleic acid.

The term “reductive environment” refers to conditions under which chemical reduction reactions occur. The referred reductive environment includes but is not limited to the presence of sodium cyanoborohydride, sodium borohydride, pyridine borane, or sodium ascorbate.

The terms “halo” and “halogen” refer to all halogens, such as chloro, fluoro, bromo, and iodo groups.

The term “alkyl” refers to a single bond chain of hydrocarbons ranging from 1 to 20 carbon atoms and includes but is not limited to methyl, ethyl, propyl, isopropyl, n-butyl, sec-butyl, isobutyl, tert-butyl, pentyl, isoamyl, hexyl, octyl, and dodecanyl groups.

The term “O-alkyl” denotes the group-OR, where R is an alkyl, and includes but is not limited to O-methoxy, O-ethoxy, O-propoxy, O-isopropoxy, O-butoxy, O-isobutoxy, and O-pentoxy groups.

The terms “ethylenediamine motif”, “EDA motif”, “EDA group”, and “EDA” refer to a chemical motif of R—CH₂—NH—(CH₂)₂—NH₂, where R is any substituent. Structure of the EDA motif may as well be recognised in an ionic form or in a form of a salt.

The term “reactive functional group” refers to any functional group that is incorporated at the RNA 5′ or 3′ end that can react with another (same or different) reactive group in a selective manner in presence of other (natural or unnatural) chemical moieties contained within structure of the circRNA precursor. The representative pairs of 5′ and 3′ reactive groups which are reactive towards each other, include but are not limited to EDA and dialdehyde, hydrazide and dialdehyde, azide and alkyne, azide and cyclooctyne derivative, amino and carboxylic group, amino and fluorosulfonylgroup, azide and phosphine, etc.

The terms “nucleic acids alcohol precipitation”, “nucleic acids precipitation”, and “alcohol precipitation” refer to the process of nucleic acid isolation. It is a technique to desalt and concentrate nucleic acids from aqueous solutions. Addition of polar organic solvents (e. g. ethanol, isopropanol, acetone) and salts (e. g. sodium acetate, ammonium acetate, lithium chloride, sodium perchlorate, lithium perchlorate) to nucleic acid solution causes the precipitation of nucleic acids.

The terms “polyacrylamide gel electrophoresis” and “PAGE” refer to a technique used for separation of biological macromolecules according to their electrophoretic mobility. Electrophoretic mobility is a function of the mass, conformation, topology, and charge of the molecule. When PAGE is performed in denaturing conditions, it provides information on the composition of the nucleic acid species in the sample.

The terms “reversed-phase high pressure chromatography”, “RP-HPLC, and “HPLC” refer to a technique for separation, identification, quantification, and isolation of components of a sample mixture. It relies on pumps constituting a flow of pressurized liquid mobile phase (a mixture of solvents and solutions) through a solid matrix of the stationary phase. Components of the sample interact differently with mobile and stationary phases and are in equilibrium between adsorbed and solubilized states. This is facilitated by a combination of hydrophobic, dipole-dipole and ionic interactions. As chromatography progresses, the analytes are resolved based on their elution velocity, can be detected by their molecular properties, and collected into separate fractions (peaks). The elution velocity of individual components of the sample is parameterized by the retention time of the fraction and the peak shape. The active component of the stationary phase is a monolith or particle solid resin of organic and/or inorganic composition, such as silica, poli (styren-co-divinylbenzen), or other polymers. The mobile phase is an aqueous or organic solution or a mixture of aqueous and organic solutions. The chromatography may be performed in ion-pair mode, in elevated temperatures, and for analytical as well as preparative purposes.

The terms “translation in vitro”, “protein synthesis in vitro”, and “protein expression in vitro” refer to a process characterised as an enzymatic production of polypeptides based on sequence of RNA or RNA analog in an artificial system. An in vitro system includes but is not limited to artificial reaction mixture, cellular lysates, nuclear lysates, cell cultures, and tissue cultures.

The terms “translation in vivo”, “protein synthesis in vivo”, and “protein expression in vivo” refer to a process characterised as an enzymatic production of polypeptides based on sequence of RNA or RNA analog in a natural system. An in vivo system includes but is not limited to whole organisms, organs, cellular and tissue transplants of human, animal, plant, fungal, or bacterial origin.

The terms “therapeutic protein” or “therapeutic proteins” refer to a polypeptide molecule that may be utilized in the course of therapy or medical studies. The classes of therapeutic proteins include but are not limited to antigens, antibodies, immunogenic proteins, fluorescent proteins, reporter proteins, protein hormones, membrane proteins, transcription factors, regulatory proteins, systems for gene editing and systems for gene regulation.

The terms “therapeutic gene” or “therapeutic genes” refer to a sequence of a nucleic acid containing an open-reading frame encoding the amino acid sequence of the therapeutic protein. Therapeutic genes may consist of but are not limited to sequences encoded in natural or synthetic viral DNA or RNA, plasmid vectors, RNA, mRNA or circRNA.

The term “pharmaceutical composition” refers to a composition of substances comprising of active substances and helper substances, such as pharmaceutically acceptable solvents and/or carriers. Pharmaceutical composition may be formulated as a solid, liquid, aqueous solution, aqueous emulsion, suspension, lipid nanoparticles, liposomes, or liposomal suspension. The term pharmaceutical composition includes but is not limited to compositions designed for expression of a therapeutic genes and proteins in in vitro and in vivo setup.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows the general concept of the preparation of circRNA analogs comprising 7-methylguanosine (exemplified using EDA-linker-Cap1). A) First, the precursor of circRNA analog (FG-linker-RNA) is synthesized during an in vitro transcription (IVT) reaction. Due to the presence of a transcription initiator (FG-linker-AG), the transcribed RNA contains a reactive functional group (FG) at the 5′ end. Next, the 3′ end reactive functional group is incorporated into RNA post-synthetically, by either enzymatic reaction or, preferably, chemical reaction. The 5′ and 3′ reactive functional groups are reactive towards each other either spontaneously or in the presence of a specific trigger. B) As an example, a precursor of circRNA analog (EDA-linker-RNA or EDA-linker-Cap1-RNA) is synthesized during IVT. Due to the presence of a transcription initiator EDA-linker-AG or EDA-linker-Cap1, the transcribed RNA contains an ethylenediamine (EDA) moiety at the 5′ end. The precursor is first chemically modified at the 3′ end by treatment with periodate. This causes selective oxidation of the 3′ ribose, which is prone to nucleophilic attack by the EDA moiety. Finally, the addition of a reductive agent (i.e. NaBH₃CN) leads to an irreversible covalent linkage of RNA's 5′ and 3′ ends.

FIG. 2 shows a comparison of enzymatic and chemical circularization. 5′-modified RNA (pre-circRNA2) was used as a precursor for chemical circularization. As a reference, 5′-monophosphorylated RNA of the same sequence (pre-circRNA1) was used as a substrate in enzymatic circularization catalyzed by T4 RNA ligase I. The substrates and products of chemical and enzymatic reactions were analyzed by PAGE (6% AA, AA:MBAA=19:1, 7 M Urea, 1×TBE). The yields of the reactions were determined by densitometry. M refers to linear RNA length marker. HMW refers to linear RNA of length ranging from 1500 nt to 6000 nt.

FIG. 3 shows an RNase R-mediated removal of linear RNA in the presence of circular RNA. Samples of a linear precursor of circRNA analog (pre-circRNA2) and products of chemical circularization (a mixture of pre-circRNA2 and circRNA2) were treated with a processive 3′ exonuclease (RNase R) that selectively hydrolyses linear RNA species. After RNase R treatment the samples were analyzed by PAGE (6% AA, AA:MBAA=38:1, 7 M Urea, 1×TBE). M refers to linear RNA length marker. HMW refers to linear RNA of length ranging from 2000 nt to 6000 nt.

FIG. 4 shows a RNase H digestion assay to confirm the circular topology of chemically circularized RNAs. Samples of a circRNA analog (circRNA2) and its linear precursor (pre-circRNA2) underwent DNA-dependent endonucleolytic cleavage catalyzed by RNase H and subsequent PAGE. In presence of targeting DNA oligonucleotide, the circRNA analog is nicked. As a result, nicked circRNA exhibits electrophoretic mobility of a linear precursor. On the other hand, cleavage of the linear precursor leads to two RNA molecules of lower size. PAGE conditions: 6% AA, AA:MBAA=38:1, 7 M Urea, 1×TBE. M refers to linear RNA length marker. HMW refers to linear RNA of length ranging from 2000 nt to 6000 nt.

FIG. 5 shows a polyacrylamide gel electrophoresis of circRNA-influence of gel crosslinking degree. Samples of a RNA length marker, a linear precursor of circRNA analog (pre-circRNA2), and products of chemical circularization (a mixture pre-circRNA2 and circRNA2) were analyzed by PAGE. Two types of 6% polyacrylamide gels were used in parallel: one with higher crosslinking degree (AA:MBAA=19:1, 7 M Urea, 1×TBE) and one with lower crosslinking degree (AA:MBAA=38:1, 7 M Urea, 1×TBE). The migration of circRNA is slower than that of linear RNA. Electrophoretic mobility differences are pronounced in gel with a higher crosslinking degree. HMW refers to linear RNA of length ranging from 2000 nt to 6000 nt.

FIG. 6 shows an optimization of RNA conformation using complementary DNA oligonucleotides. A) Prediction of minimum free energy structure of RNA5 sequence generated with RNAfold web server. B) Prediction of tertiary structure of an RNA sequence comprising flanking regions of RNA5 sequence generated with RNAComposer web server. The predictions indicate, that 3′ poliA sequence may distance 5′ and 3′ ends and hinder any reaction between them. Fluorescence spectra of Cy5-RNA5-Cy3 probe recorded at 4° C. (C) or 20° C. (D) before (w/o ON) or after hybridization with one of the complementary DNA oligonucleotides (ON2-ON5). Emergence of 660 nm emission band indicates the occurrence of the FRET phenomenon facilitated by decreased distance between 5′ and 3′ ends.

FIG. 7 shows the influence of complementary DNA oligonucleotides on chemical circularization. Pre-circRNA10 was used as a precursor for chemical circularization. Before the oxidation step, the precursor was annealed with one of the complementary DNA oligonucleotides (ON2-ON5). After the reaction, the samples were analyzed by PAGE (6% AA, AA:MBAA=38:1, 7 M Urea, 1×TBE). As a reference, samples of precursor before reaction (NR) and products of reaction without complementary DNA (w/o ON) were included. M refers to linear RNA length marker. HMW refers to linear RNA of length ranging from 3000 nt to 6000 nt. The relative amounts of circular product (P), linear dimer (D), and unreacted substrate(S) were determined by densitometry.

FIG. 8 shows an attempted separation of linear and circular RNA using IE-HPLC (A), SEC-HPLC (B) and RP-HPLC (C). A sample containing a mixture of linear and circular RNA (pre-circRNA2+circRNA2) was chromatographed using three different modes and columns. As a reference, linear RNA (pre-circRNA2) was resolved in same conditions. IE-HPLC conditions: CIMac PrimaS™ 0.1 mL Analytical Column (2 μm); A—50 mM HEPES pH 7.0; B—200 mM sodium pyrophosphate, 50 mM HEPES pH 8.5; 0% B for 2 min, 0-100% B in 6 min, 100% B for 2 min, 100-0% B in 2 min, 0% B for 1 min; flow 1.0 ml/min at 25° C. SEC-HPLC conditions: Yarra 3 μm SEC-4000 300×7.8 mm; A—100 mM Na₂HPO₄, 0.025% NaN₃ pH 6.8; 100% A for 50 min; flow 1.2 ml/min at 25° C.; RP-HPLC conditions: RNASept Prep C18 50×7.8 mm 2 μm; A—100 mM HAA, 10% MeCN, pH 7.0; B—100 mM HAA, 75% MeCN, pH 7.0; 20-70% B in 25 min; flow: 1.0 ml/min at 60° C.

FIG. 9 shows the purification of circRNA using reversed-phase high-pressure liquid chromatography. Products of chemical circularization of pre-circRNA4 were chromatographed under denaturing conditions (60° C., hexylammonium acetate buffer and acetonitrile mixture as a mobile phase). The UV-absorbing eluate was collected and separated into fractions that corresponded to different peaks of absorbance. Next, the fractions were concentrated and analyzed by PAGE, along with EP5-GLuc before and after circularization. NR-precursor (pre-circRNA4), Cr-crude products of circularization, F1-F3-concentrated HPLC fractions, M-linear RNA length marker. HMW refers to linear RNA of length ranging from 3000 nt to 6000 nt. PAGE conditions: 6% AA, AA:MBAA=38:1, 7 M Urea, 1×TBE. Purified circRNA is in fraction 2.

FIG. 10 shows the influence of mobile phase composition on RP-HPLC of circRNA. A mixture of linear (pre-circRNA14) and circular (circRNA14) RNAs was resolved with RP-HPLC. Each chromatography was performed in presence of different ion-pairing agent: A) hexylammonium acetate; B) triethylammonium acetate; C) 2-ethylhexylammonium acetate; D) dibutylammonium acetate; E) N,N-dimethylhexylammonium acetate; F) N,N-diisopropylammonium acetate.

FIG. 11 shows a PAGE analysis of selected circRNA precursors and circRNA after HPLC and RNase R treatment. M-linear RNA length marker. HMW refers to linear RNA of length ranging from 3000 nt to 6000 nt. PAGE conditions: 6% AA, AA:MBAA=38:1, 7 M Urea, 1×TBE.

FIG. 12 shows the translational activity of circRNA analogs in vitro. HEK 293T cells were transfected with unmodified i.e. enzymatically obtained circRNA (circRNA11) or modified, i.e. chemically obtained circRNA (circRNA12 with unnatural linkage, and circRNA13 with a cap structure) encoding Gaussia luciferase, a reporter protein secreted by the cells to the medium. The circRNA precursors (pre-circRNA11-13) were used as a references. The cell media were harvested at timepoints (4, 24, 48, 72, 96, 120, 144 and 168 hours post transfection) and used for quantification of the reporter protein levels with luciferase bioluminescence assay. The detected luminescence from all timepoints was summarized to give the total luminescence. The data presented in the graph derives from tree independent replicates, error bars represent a standard error of the mean (SEM).

FIG. 13 shows the translational activity of circRNA analogs in vitro (continued). HEK 293T cells were transfected with unmodified (circRNA11) or modified circRNA (circRNA12, 13) encoding Gaussia luciferase, a reporter protein secreted by the cells to the medium. The circRNA precursors (pre-circRNA11-13) were used as a reference. The cell media were harvested at timepoints (4, 24, 48, 72, 96, 120, 144 and 168 hours post transfection) and used for quantification of the reporter protein levels with luciferase bioluminescence assay. The data presented in the graph derives from tree independent replicates, error bars represent a standard error of the mean (SEM).

FIG. 14 shows the translational activity of circRNA analogs in vitro. A549, HEK 293T, Hep G2, HeLa, mouse dendritic cells as well as macrophages (established from bone marrow monocytes) were transfected with unmodified (circRNA11) or modified circRNA (circRNA13) encoding Gaussia luciferase, a reporter protein secreted by the cells to the medium. The circRNA precursors (pre-circRNA11 and pre-circRNA13) as well as mock transfection (water instead of RNA) were used as a reference. Cell media were harvested at timepoints (4, 24, 48, 72, 96, 120, 144 and 168 hours post transfection or 4, 24, 48, 72, 96 and 120 hours post transfection as in case of dendritic cells) and used for quantification of the reporter protein levels with luciferase bioluminescence assay. The detected luminescence from all timepoints was summarized to give the total luminescence that was subsequently normalizes to the average luminescence obtained for circRNA11. The data presented in the graph derives from tree independent replicates, error bars represent a standard error of the mean (SEM).

FIG. 15 shows the translational activity of circRNA analogs in vitro. A549, HEK 293T, HeLa, Hep G2 were transfected with unmodified (enzymatically obtained) circRNA (circRNA23, circRNA26), modified circRNA obtained from a precursor carrying EDA-PEG₅-AG at the 5′ terminus (circRNA12, circRNA24 and circRNA27) or modified circRNA obtained from a precursor carrying EDA-PEG₅-Cap1 at the 5′ terminus (circRNA13, ciercRNA25 and circRNA28) encoding Gaussia luciferase, a reporter protein secreted by the cells to the medium. The ctrl transfection (water instead of RNA) was used as a reference. The cell media were harvested at timepoints 4, 24, 48, 72, 96, 120, 144 and 168 hours post transfection) and used for quantification of the reporter protein levels with luciferase bioluminescence assay. The detected luminescence from all timepoints was summarized to give the total luminescence that was subsequently normalized to the average luminescence obtained for linRNA33. The data presented in the graph derives from tree independent replicates, error bars represent a standard error of the mean (SEM).

FIG. 16 shows the translational activity of circRNA analogs in vitro (continued). A549 cells were transfected with modified circRNAs with EDA-PEG₅-Cap1, Cap-MW3 and Cap-AR67 at 5′ terminus (circRNA 25, 31, 32) encoding Gaussia luciferase, a reporter protein secreted by the cells to the medium. The circRNA precursors (pre-circRNA25, 31, 32) were used as a reference. The cell media were harvested at timepoints (4, 24, 48, 72, 96, 120, 144 and 168 hours post transfection) and used for quantification of the reporter protein levels with luciferase bioluminescence assay. The data presented in the graph derives from tree independent replicates, error bars represent a standard error of the mean (SEM).

FIG. 17 shows the translational activity of circRNA analogs in vitro. A549, HEK 293T, HeLa, Hep G2 were transfected with modified circRNAs obtained from precursors carrying EDA-PEG₅-Cap1, Cap-MW3, or Cap-AR67 at the 5′ terminus (circRNA25, circRNA31 and circRNA32) encoding Gaussia luciferase, a reporter protein secreted by the cells to the medium. The ctrl transfection (water instead of RNA) was used as a reference. The cell media were harvested at timepoints (4, 24, 48, 72, 96, 120, 144 and 168 hours post transfection) and used for quantification of the reporter protein levels with luciferase bioluminescence assay. The detected luminescence from all timepoints was summarized to give the total luminescence. The data presented in the graph derives from tree independent replicates, error bars represent a standard error of the mean (SEM).

FIG. 18 shows the translational activity of circRNA analogs in vitro. A549, HEK 293T, HeLa, Hep G2 were transfected with unmodified and modified circRNAs obtained from precursors carrying 5-monophosphate, EDA-PEG₅-AG, EDA-PEG₅-Cap1, Cap-AR67, or Cap-AR^{L03} at the 5′ terminus (circRNA7, circRNA7A, circRNA8, circRNA34 and circRNA35) encoding Gaussia luciferase, a reporter protein secreted by the cells to the medium. The ctrl transfection (water instead of RNA) was used as a reference. The cell media were harvested at timepoints 4, 24, 48, 72 hours (post-transfection) and used for quantification of the reporter protein levels with luciferase bioluminescence assay. The detected luminescence from all timepoints was summarized to give the total luminescence. The data presented in the graph derives from tree independent replicates; error bars represent a standard error of the mean (SEM).

FIG. 19 shows the translational activity of circRNA analogs in vitro (continued). HEK 293T cells were transfected with N1-methylpseudouridine (m¹ψ) unmodified (circRNA18A-m¹ψ) or modified circRNA (circRNA30-m¹ψ) encoding Gaussia luciferase, a reporter protein secreted by the cells to the medium. The unmodified circRNA with uridine (circRNA18A) and modified circRNA with uridine (circRNA30) were used as a reference. The cell media were harvested at timepoints (4, 24, 48, 72, 96, 120, 144 and 168 hours post transfection) and used for quantification of the reporter protein levels with luciferase bioluminescence assay. The data presented in the graph derives from tree independent repelicates, error bars represent a standard error of the mean (SEM).

FIG. 20 shows the translational activities of circRNA analogs in an in vitro cell-free system. Flexi Rabbit Reticulocyte Lysate was incubated for 1 h with a mixture of circRNAs (unmodified circRNA29 or modified circRNA30) and cap-dependent translation inhibitor (m⁷GpsppG dinucleotide). The reaction was immediately placed into a microplate reader (Synergy H1, BioTek). 50 μL of h-coelenterazine in 1×PBS was added and the luminescence was measured at 25° C. The assay was repeated to calculate the and mean and standard error (SEM).

### DETAILED DESCRIPTION

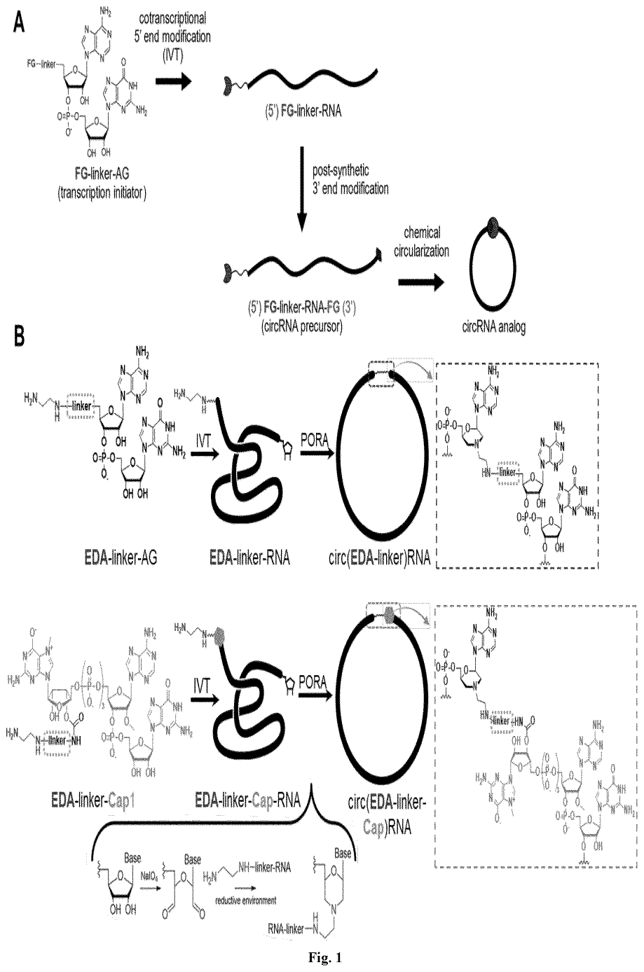

The present invention relates to circular RNA (circRNA) analogs obtained by circularization of a circRNA precursor containing two reactive functional groups, one at the 5′-end and a second at the 3′-end. [FIG. 1A] The reactive functional group at the 5′-end is incorporated into RNA during enzymatic synthesis (i.e. in vitro transcription reaction). The 3′-end reactive functional group is incorporated into RNA post-synthetically, by either enzymatic reaction or, preferably, by chemical reaction. The 5′ and 3′ reactive groups are reactive towards each other either spontaneously or in the presence of a specific trigger, such as the addition of a chemical reagent, addition of a catalysts, a change of temperature, the presence of light, or a change in the pH of the solution, etc. Representative pairs of 5′ and 3′ reactive groups reactive towards each other include, but are not limited to: EDA and dialdehyde, hydrazide and dialdehyde, azide and alkyne, azide and cyclooctyne derivative, amino and carboxylic group, amino and fluorosulfonylgroup, azide and phosphine, etc.

A representative example of a functional group at the 5′-end is the EDA motif (EDA-linker-RNA). [FIG. 1B] The precursor is a modified nucleic acid that can be prepared by in vitro transcription. The 5′-EDA motif can be readily installed during the in vitro transcription (IVT) reaction if the transcription is carried out in the presence of an EDA-modified transcription initiator [see Table 1 for examples]. This is beneficial, as unlike other methods of chemical circularization, the precursor's length is not limited by the efficiency of RNA solid-phase synthesis. The 5′-EDA motif and the nucleic acid chain of the EDA-linker-RNA are joined by a linker. The linker itself may be a linear or branched combination of simple chemical moieties (such as carbohydrate or PEG linkers), or it may contain more complex elements, such as detectable labels or a 7-methylguanosine cap analog. The circRNA precursor can bear additional modifications, such as detectable labels or 7-methylguanosine cap analogs, as long as they do not interfere with the circularization reaction. This is beneficial, as in contrast to enzymatic circularization, the user is not restricted by the activity and substrate-specificity of a particular enzyme. The chemical circularization reaction is a two-step process. First, the circRNA precursor is selectively oxidized with periodate at the 3′ end to introduce a reactive functional group (a dialdehyde) at the 3′ end of RNA. This occurs via ring-opening oxidation and the formation of a reactive acyclic dialdehyde derivative of the terminal 3′-end ribose. During the second step, the 3′-end dialdehyde derivative spontaneously reacts with the 5′ EDA motif. This step requires a reductive environment for optimal performance. If a reductive environment is ensured during this reaction, the reduction of the formed intermediate occurs, making the whole process irreversible. Except for the presence of the 5′-EDA motif (5′ reactive group) and the 3′ dialdehyde motif (3′ reactive group), the chemical circularization has no requirements concerning the chemical structure or the sequence of the circRNA precursor. This is beneficial, as in contrast to circularization techniques based on activity of catalytic nucleic acids (PIE and Tornado systems), the invented chemical circularization does not require sequence engineering and construct cloning for the preparation of the circRNA precursor.

circRNA precursors containing EDA motif were prepared by means of IVT and subsequently circularized [Table 1]. As a comparison, precursors bearing a 5′ monophosphate moiety and an identical sequence were subjected to enzymatic circularization catalysed by T4 RNA ligase I or T4 RNA ligase II. Unexpectedly, the yield of chemical circularization, in comparison to enzymatic circularization, is less dependent on the precursor sequence and is often more efficient [FIG. 2].

The circular topology of the circRNA analogs obtained by chemical circularization can be verified using exonucleolytic digestion assays, endonucleolytic assays, as well as electrophoretic mobility assays.

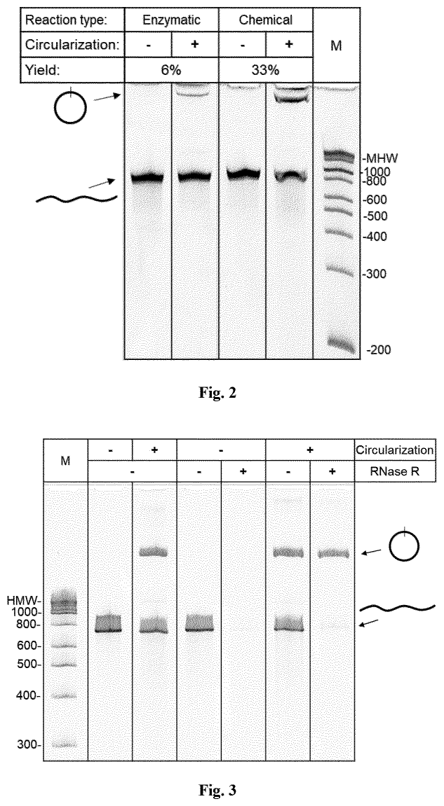

Exonucleolytic digestion assays allow one to distinguish linear RNAs from circular RNAs, as exonucleases can hydrolyse only RNA molecules that contain free 5′ or 3′ end. On the other hand, circular RNAs are resistant to exonucleolytic hydrolysis as they lack free 5′ and 3′ ends. RNase R is a processive 3′ exonuclease, that is most commonly used for such assay.³ The products of chemical circularization were treated with RNase R [FIG. 3]. In contrary to the precursors, the circRNA analogs were resistant to RNase R hydrolysis.

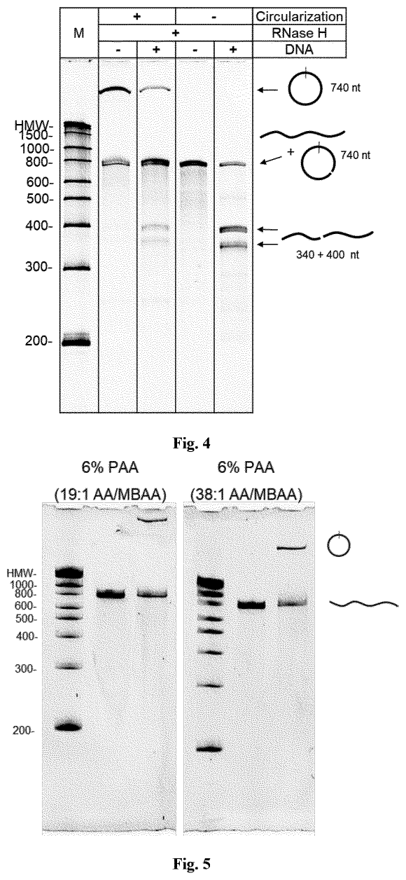

Endonucleolytic assays are based on a phenomenon of nicking and linearization of circRNA. Site-selective and sequence-dependent cleavage of a circular RNA molecule leads to a single linear RNA molecule of equal size. On the other hand, cleavage of a linear RNA molecule leads to two linear RNA molecules of smaller sizes. Such assay can be performed with RNase H, an endonuclease that nicks RNA in RNA-DNA duplexes.²⁵ RNase H and targeting DNA oligonucleotides were used for nicking of the products of chemical circularization [FIG. 4]. The assay confirmed that, in contrast to the precursors, nicking of the circRNA analogs leads to their linearization.

Electrophoretic mobility of nucleic acids is affected by their size, charge, conformation, and topology.^{26, 27} Thanks to that, circRNA and their precursors can be separated during polyacrylamide gel electrophoresis in denaturing conditions [FIG. 5]. The crosslinking degree of the polyacrylamide gel matrix is a crucial factor for the separation of RNA based on their topology. Electrophoretic separation of linear and circular RNA using a cross-linked polyacrylamide gel is significantly better than with regular or commercially available agarose gels.²⁷

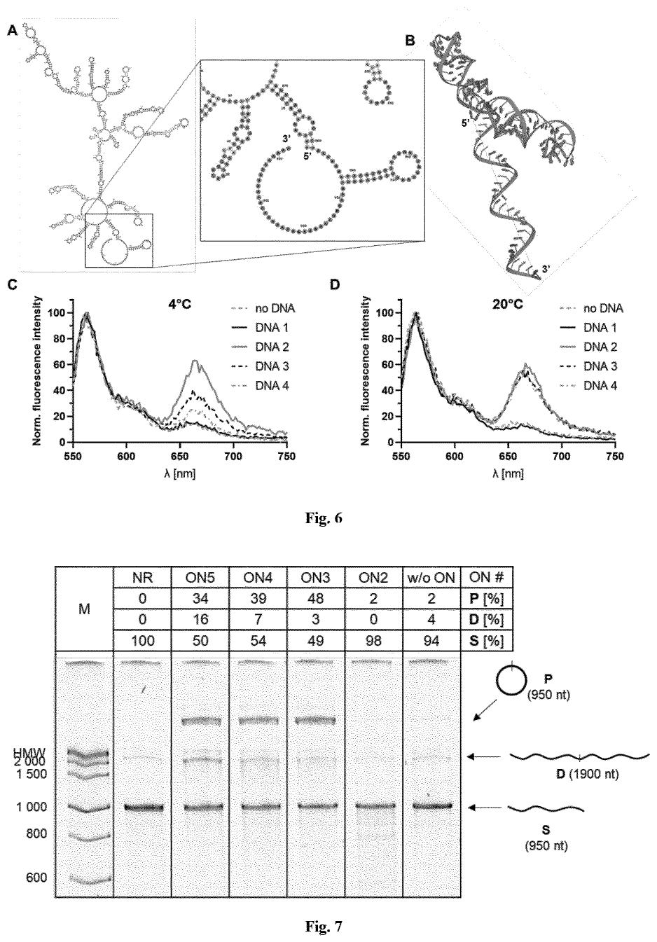

The circularization efficiency is affected by the sequence of the circRNA precursor [Tab. 2]. In some cases, such as sequences containing a 3′ poliA sequence, the circularization yield can be increased by the addition of a complementary nucleic acid oligomer [Tab. 3]. During the reaction, the oligomer and precursor hybridize and affect the conformation of the precursor. Using an oligomer with an optimal design, the conformational change of the precursor decreases the distance between the 5′ and 3′ ends [FIG. 6]. In consequence, the optimal distance and orientation of the 5′ and 3′ ends increase the circularization yield [FIG. 7].

The present invention also relates to methods of preparation and isolation of circRNA analogs. This includes development of chromatographic methods of linear and circular RNA resolution.

Previously reported methods of chromatography of circularization products were based on size-exclusion chromatography in an HPLC setup (SEC-HPLC).^{11, 12, 14} This method allows one to separate sample components based on hydrodynamic radius and is considered to provide one of the mildest chromatographic conditions. SEC-HPLC was used to resolve the products of PIE-based circularization.^{11, 12, 14} In these cases, the precursor and circRNA significantly differ in molecular size between each other, as well as form side products (such as free introns). In case of synthetic polymers (such as polystyrene or poli (ethyneneoxide)) the hydrodynamic radius (Stokes radius) is decreased upon circularization.²⁸ This property was used to separate linear and cyclic polymers using gel permeation chromatography (GPC). However, the decrease in the Stokes radius of an RNA molecule caused by its circularization has not been documented. One could speculate that intramolecular interactions, that contribute to the existence of a landscape of RNA conformers, may diminish the influence of topological change on the differentiation of Stokes radii of circular and linear RNA.²⁹ This could also explain why SEC-HPLC under mild conditions leads to the coelution of intact and nicked circRNA.^{12, 14}

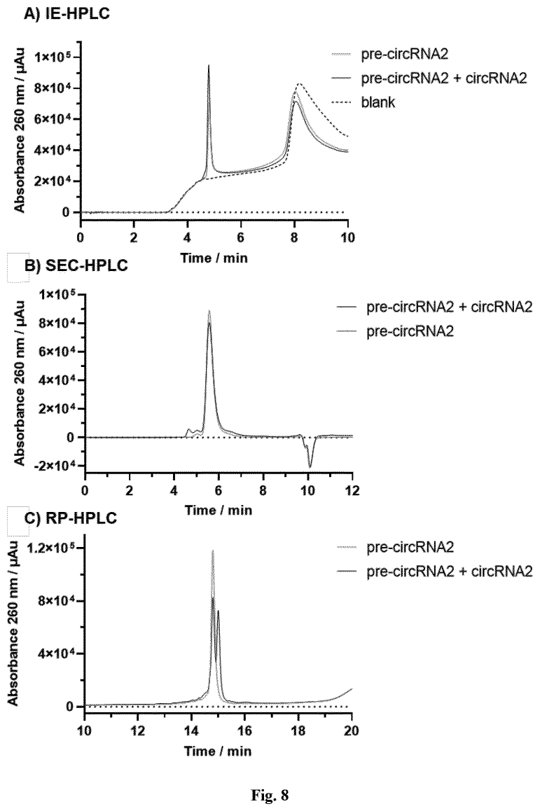

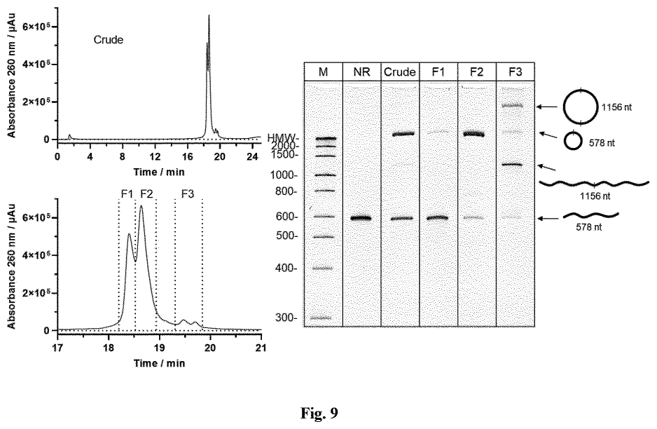

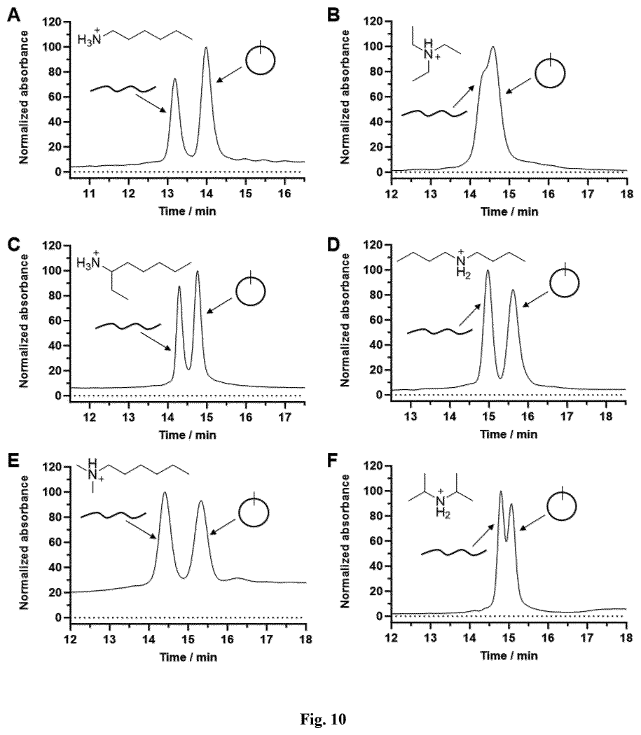

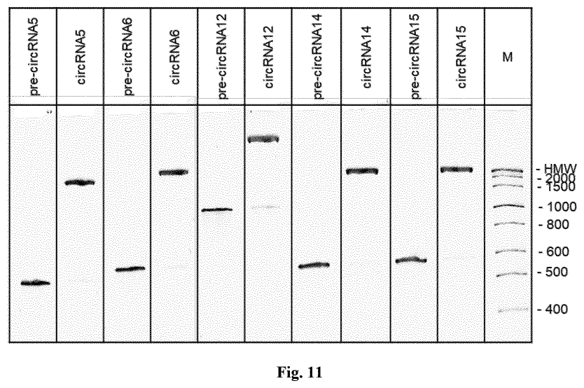

During the development of the invention, we found that both size-exclusion chromatography (SEC-HPLC) and ion-exchange HPLC (IE-HPLC) are insufficiently effective for separating linear and circular RNA of the same molar mass [FIG. 8]. Reversed-phase HPLC (RP-HPLC) in ion-pair mode was found to be the most suitable method for the separation of circularization products [FIG. 8, 9]. Chromatography was performed using polistyren-divinylbenzen (PSDVB) resins, elevated temperature (50-80° C.), and a mixture of an aqueous solution of ion pair reagent with acetonitrile as a mobile phase. The separation of linear and circular RNA is affected by composition of the mobile phase, namely the chosen ion pair reagent. The most effective resolution was obtained with 100 mM n-hexylammonium acetate (HAA), with a linear gradient of acetonitrile (45-58.5% in 25 min, flowrate 1.00 ml/min at 60° C.) [FIG. 10].

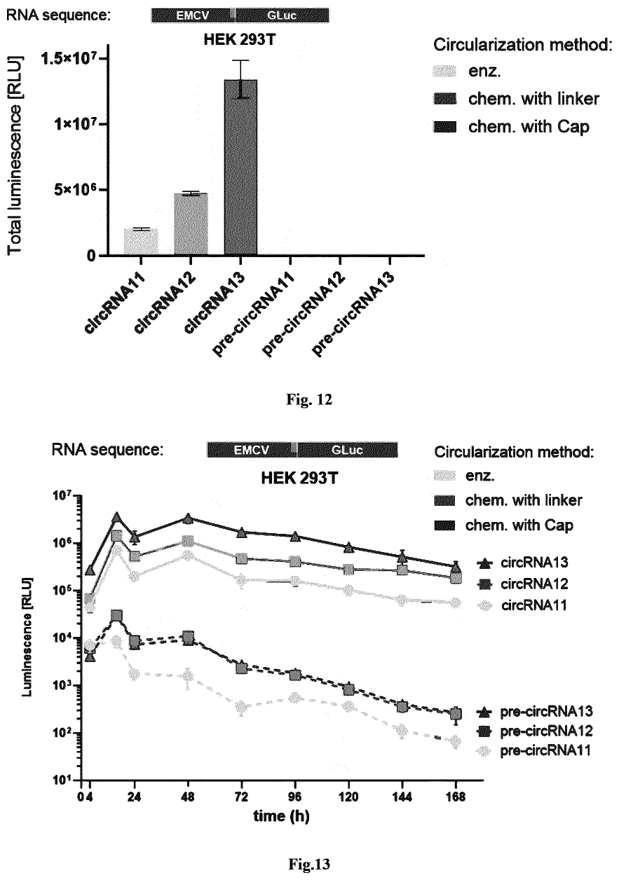

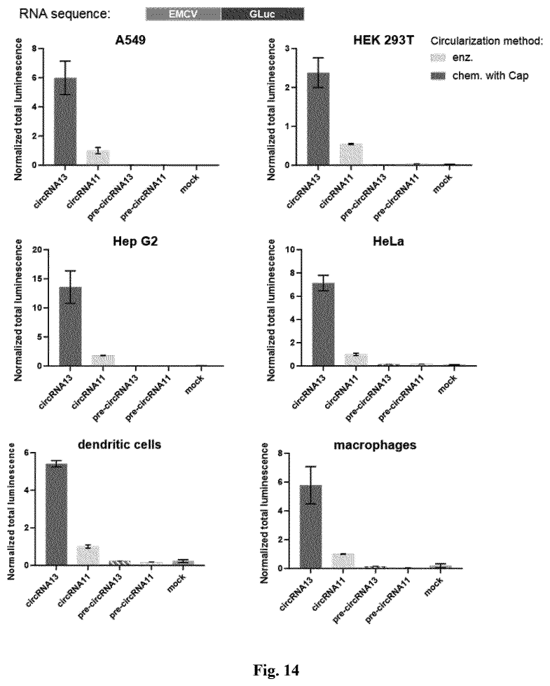

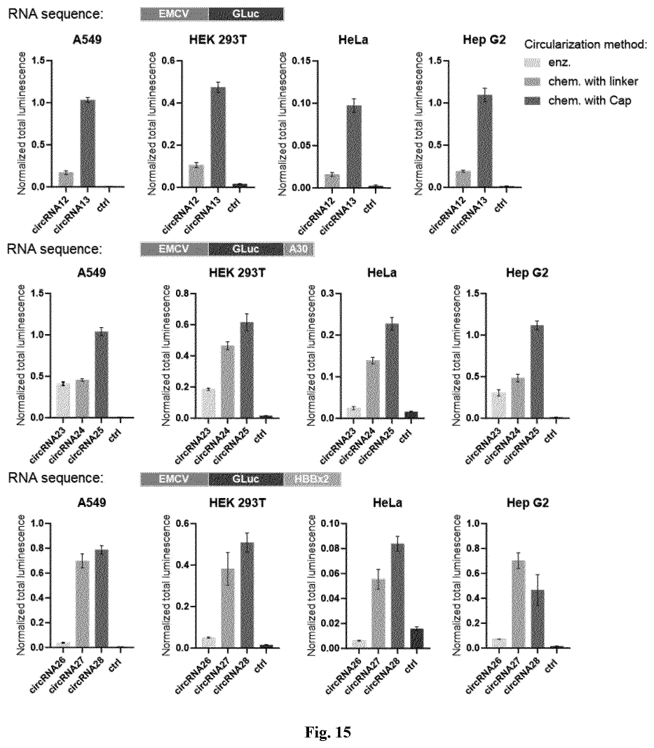

The present invention also relates to therapeutic application of circRNA analogs. To demonstrate the applicability of protein-encoding circRNA analogs in gene delivery, circRNA analogs encoding a reporter protein, Gaussia luciferase, were synthesized and expressed in cells. The levels of the reporter protein were higher in the case of expression of chemically modified circRNA analogs (circRNA12, 13) than unmodified circRNA (circRNA11) [FIG. 12]. Both modified and unmodified circRNAs exhibited increased duration of protein expression in comparison to their linear precursors (pre-circRNA11-13) [FIG. 13]. This result shows that chemical modification within the circRNA analogs is beneficial in the context of therapeutic protein expression. To better demonstrate the applicability of the invention, a broader range of mRNA sequences was investigated. The unmodified circRNAs (circRNA23, circRNA26), circular RNAs modified with a chemical linker (circRNA12, circRNA24 and circRNA27), and with modified Cap1 structure (circRNA13, circRNA25, circRNA28) were tested. These variants featured diverse sequence elements, including but not limited to a 3′-UTR comprising a tandem sequence of HBB and a polyA tract. Notably, across all instances, products resulting from chemical circularization exhibited augmented translation efficiency, as illustrated in FIG. 15.

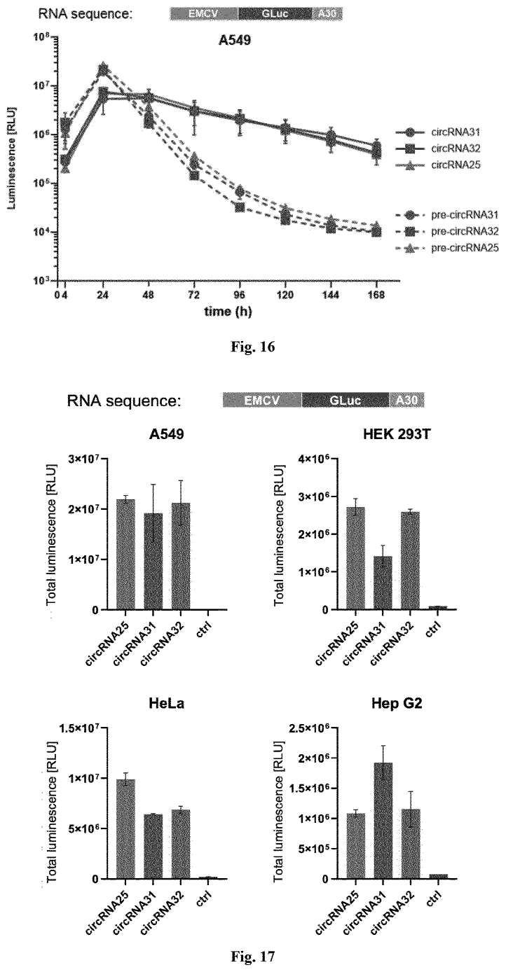

To investigate whether the position of the linker connecting the 5′-cap structure to mRNA 3′ end has an influence on the translational activity of coding cricRNAs, circRNA analogs modified with one of three different caps (EDA-PEG₅-Cap1, Cap-MW3, and Cap-AR67) and encoding protein Gaussia luciferase were synthesized and transfected into cells. All modified circRNAs (circRNA25, 31, 32) exhibited prolonged protein expression in comparison to their linear precursors (pre-circRNA25, 31, 32) [FIG. 16]. Furthermore, in vitro experiments showed that the expression levels of the reporter protein were comparable for all of chemically modified circRNA analogs (circRNA25, 31, 32) [FIG. 17]. These results show that circRNA analogs with various chemically modified structures are beneficial in the context of therapeutic protein expression.

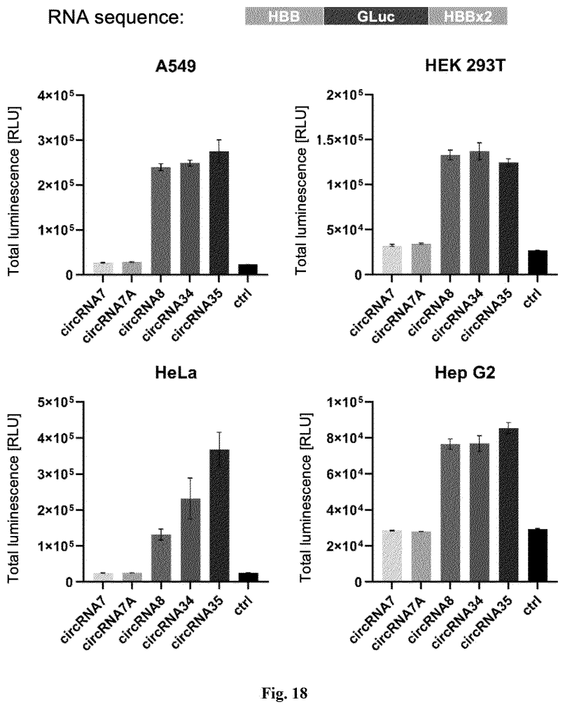

To investigate the influence of a linker connecting the 5′-cap structure to the mRNA 3′ end on the translational activity of coding cricRNAs, circRNA analogs modified with an EDA-PEG₅ linker, one of three different caps (EDA-PEG₅-Cap1, Cap-AR67, and Cap-AR^{L03}), and unmodifier circRNA and encoding protein Gaussia luciferase were synthesized and transfected into cells. All modified circRNAs (circRNA8, 34, 35) exhibited higher protein expression level compared to unmodified circRNA7 and modified RNA7A [FIG. 18]. These results show that circRNA analogs with various chemically modified structures are beneficial in the context of therapeutic protein expression.

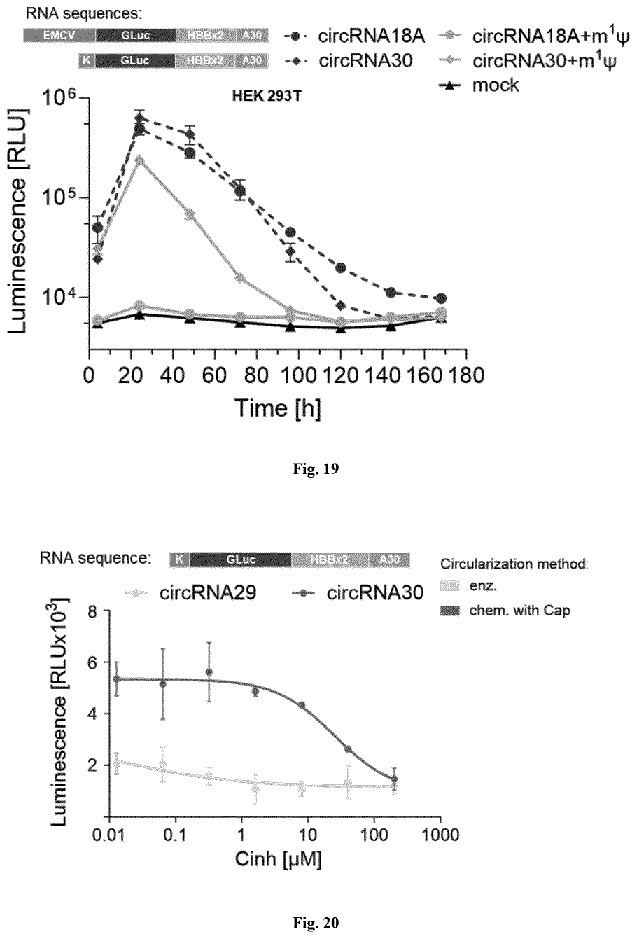

N1-methylpseudouridine (mly) is a pivotal modification that reduces the unwanted reactogenicity of therapeutic mRNA. However, the integration of mly and other noncanonical nucleobases carries the potential to disrupt the functionality of RNA elements, such as Internal Ribosome Entry Site (IRES) or autocatalytic sequences necessary for RNA circularization (PIE method), by inducing alterations in their secondary structure. In light of this, we tested whether an endocyclic cap structure could serve as a viable substitute for the IRES element, thereby facilitating the translation of circular RNA (circRNA) harboring the m′y base modification. To this end, the linear precursors were synthesized through in vitro transcription (IVT) in the presence of mly-triphosphat and subjected to either chemical or enzymatic circularization. Following standard procedures, the resulting circRNAs were transfected into HEK cells. We found that, circRNA30+m¹ψ exhibited only a marginal reduction in translational activity compared to its m¹ψ-free counterpart, as illustrated in FIG. 19. This indicated that the circRNAs according to the present inventio are the first circRNAs compatible with mRNA body modifications such as m¹ψ.

In order to validate the cap-dependent translation mechanism of the capped circRNA analogs, an assessment was conducted by comparing the expression levels of unmodified circRNA29 and modified circRNA30 in a cell-free system (rabbit reticulocyte lysate, RRL) in the presence of a cap-dependent translation inhibitor, namely m⁷GpsppG dinucleotide. Notably, an escalation in the inhibitor concentration led to a reduction in the level of reporter protein resulting from the translation of circRNA30, while no significant impact was observed on the translation of circRNA29 [FIG. 20]. This indicates that the circRNAs according to the present invention, are the first circRNAs that lead to protein production via a cap-dependent mechanism.

Tables and their Brief Descriptions

Table 1: List of DNA templates (linearized plasmids) and corresponding RNA sequences with description and properties. Column description: #plasmid—designation of a plasmid; Restrictase—name of the enzyme used for linearization of the plasmid; #RNA sequence—designation of a RNA sequence encoded in the DNA template for IVT; RNA sequence elements—5′ and 3′ untranslated regions (5′ UTR and 3′ UTR), protein-coding sequence (CDS), sequence downstream of the 3′ UTR (3′ tail); RNA length-total length of the transcript; RNA E260—molar extinction coefficient calculated for particular RNA sequence.³⁰ HBB: human betaglobin UTR (5′ or 3′) sequence, EMCV: Encephalomyocarditis virus IRES, RSV: respiratory syncytial virus IRES, OligoIRES: synthetic IRES, eGFP: enhanced green fluorescent protein sequence, GLuc: Gaussia-Dura luciferase sequence, A30: 30 nucleotide-long poliadenine sequence.

| | | | | | | | RNA | |
|---|---|---|---|---|---|---|---|---|
| # | | # RNA | RNA sequence elements | | | | length | RNA E₂₆₀ |
| Plasmid | Restrictase | sequence | 5′ UTR | CDS | 3′ UTR | 3′ tail | [nt] | [μM⁻¹cm⁻¹] |
| PL1 | BamHI | RNA1 | — | eGFP | — | — | 740 | 8.93 |
| PL2 | BamHI | RNA2 | — | GLuc | — | — | 578 | 6.99 |
| PL3 | BamHI | RNA3 | HBB | | — | — | 626 | 7.58 |
| | PmeI | RNA4 | | | HBBx2 | — | 918 | 11.0 |
| | AarI | RNA5 | | | HBBx2 | A30 | 952 | 11.5 |
| PL4 | BamHI | RNA6 | EMCV | | — | — | 1152 | 13.9 |
| PL5 | | RNA7 | RSV | | — | — | 598 | 7.25 |
| PL6 | | RNA8 | OligoIRES | | — | — | 711 | 9.35 |
| PL7 | Esp3I | RNA9 | EMCV | | HBBx2 | A30 | 1446 | 17.86 |
| PL8 | AarI | RNA10 | Kozak | | HBBx2 | A30 | 908 | 10.99 |
| PL9 | Esp3I | RNA11 | EMCV | eGFP | — | pAC | 1492 | 13.51 |
| PL10 | Eco52I | RNA12 | EMCV | GLuc | spacer | | 1612 | 19.61 |
| PL11 | Esp3I | RNA13 | EMCV | | — | A30 | 1207 | 14.39 |
| PL7 | PmeI | RNA14 | EMCV | | HBBx2 | — | 1446 | 17.24 |

TABLE 2
List of circRNA and yields of their chemical or enzymatic circularization.

| | # RNA | | Circularization | DNA | Circularization |
|---|---|---|---|---|---|
| # circRNA | sequence | IVT initiator | type | oligonucleotide | yield [%] |
| circRNA1 | RNA1 | GMP | Enz. (T4 Lig. 1) | w/o ON | 5-10 |
| circRNA2 | | EDA-PEG₅- | Chem. | | 30-45 |
| | | AG | | | |
| circRNA3 | RNA2 | GMP | Enz. (T4 Lig. 1) | | 36 |
| circRNA4 | | EDA-PEG₅- | Chem. | | 60 |
| | | AG | | | |
| circRNA5 | | EDA-PEG₅- | | | 53 |
| | | Cap1 | | | |
| circRNA6 | RNA3 | EDA-PEG₅- | Chem. | | 46 |
| | | Cap1 | | | |
| circRNA7 | RNA4 | GMP | Enz. (T4 Lig. 1) | w/o ON | 13 |
| | | | Enz. (T4 Lig. 2) | w/o ON | 31 |
| | | | | ON1 | 1-45 |
| circRNA7A | | EDA-PEG₅- | Chem. | ON1 | 63 |
| | | AG | | | |
| circRNA8 | | EDA-PEG₅- | Chem. | w/o ON | 10 |
| | | Cap1 | | ON1 | 50 |
| circRNA9 | RNA5 | GMP | Enz. (T4 Lig. 1) | w/o ON | 9 |
| | | | Enz. (T4 Lig. 2) | w/o ON | 32 |
| | | | | ON4 | 61 |
| circRNA10 | | EDA-PEG₅- | Chem | w/o ON | 2 |
| | | Cap1 | | ON4 | 40 |
| circRNA11 | RNA6 | GMP | Enz. (T4 Lig. 1) | w/o ON | 10 |
| | | | Enz. (T4 Lig. 2) | ON6 | 46 |
| circRNA12 | | EDA-PEG₅- | Chem | w/o ON | 44 |
| | | AG | | ON6 | 44 |
| circRNA13 | | EDA-PEG₅- | | ON6 | 41 |
| | | Cap1 | | | |
| circRNA14 | RNA7 | GMP | Enz. (T4 Lig. 1) | w/o ON | 40 |
| | | | Enz. (T4 Lig. 2) | | 84 |
| circRNA15 | | EDA-PEG₅- | Chem | | 52 |
| | | AG | | | |
| circRNA16 | RNA8 | GMP | Enz. (T4 Lig. 1) | | 5-10 |
| circRNA17 | | EDA-PEG₅- | Chem | | 53 |
| | | AG | | | |
| circRNA18A | RNA9 | GMP | Enz. (T4 Lig. 2) | ON7 | 56 |
| circRNA18 | | EDA-PEG₅- | Chem | ON7 | 57 |
| | | Cap1 | | | |
| circRNA19 | RNA10 | EDA-PEG₅- | Chem | ON8 | 58 |
| | | Cap1 | | | |
| circRNA20 | RNA11 | GMP | Enz. (T4 Lig. 1) | ON9 | 1-3 |
| circRNA21 | | EDA-PEG₅- | Chem | | 37-51 |
| | | AG | | | |
| circRNA22 | RNA12 | NA | PIE | NA | 34-54 |
| circRNA23 | RNA13 | GMP | Enz. (T4 Lig. 2) | ON10 | 40 |
| circRNA24 | | EDA-PEG₅- | Chem | | 45 |
| | | AG | | | |
| circRNA25 | | EDA-PEG₅- | | | 57 |
| | | Cap1 | | | |
| circRNA26 | RNA14 | GMP | Enz. (T4 Lig. 2) | ON11 | 52 |
| circRNA27 | | EDA-PEG₅- | Chem | | 45 |
| | | AG | | | |
| circRNA28 | | EDA-PEG₅- | | | 35 |
| | | Cap1 | | | |
| circRNA29 | RNA10 | GMP | Enz. (T4 Lig. 2) | ON8 | 46 |
| circRNA30 | | EDA-PEG₅- | Chem | | 53 |
| | | Cap1 | | | |
| circRNA31 | RNA13 | Cap-MW3 | Chem | ON10 | 45 |
| circRNA32 | | Cap-AR67 | | | 43 |
| linRNA33 | RNA5 | Cap1 | — | — | NA |
| circRNA34 | RNA4 | Cap-AR67 | Chem | ON1 | 54 |
| circRNA35 | | Cap-AR103 | | | 56 |

Column description: # circRNA—designation of a circRNA and corresponding pre-circRNA; # RNA sequence—designation of an RNA sequence encoded in the DNA template for IVT; IVT initiator—compound used as transcription initiator in IVT reaction; Circularization type—type of reaction (chemical or enzymatic catalyzed by T4 RNA ligase I or T4 RNA ligase II) used for RNA circularization; DNA oligonucleotide—information on whether a complementary DNA oligonucleotide was used during particular circularization reaction; Circularization yield—fraction of the circRNA precursor converted to particular circRNA product during circularization reaction, determined by PAGE of crude circularization products and densitometric analysis.

TABLE 3
List of complementary DNA oligonucleotides used for RNA circularization
and FRET experiments. Column descriptions: #ON-designation of a DNA
oligonucleotide sequence; # RNA sequence-designation of an RNA sequence;
ON sequence-sequence of the designated oligonucleotide.

| # | # RNA | |
|---|---|---|
| ON | sequence | ON sequence |
| ON1 | RNA4 | AGCAAATGTCTAGATTATCCCTAACATTTAAATGCAATGAAAA |
| ON2 | RNA5 | TCCCTTTTTT |
| ON3 | | TCTAGATTATCCCTTTTTTTTTTTTTTT |
| ON4 | | GAAGCAAATGTCTAGATTATCCCTTTTTTTTTTTTTTTTTTTTTTTTT |
| ON5 | | GAAGCAAATGTCTAGATTATCCCTTTTTTTTTTTTTTTTTTTTTTTTTTTTTTTG |
| | | TTTAAACATTTA |
| ON6 | RNA6 | AGGGGGGGAGGGAGAGGGGTACCCCCTATCCTTAGTCACCACCGGCCCCCT |
| ON7 | RNA9 | GGGAGAGGGGTACCCCCTTTTTTTTTTTTTTTTTTTTT |
| ON8 | RNA10 | CTTTGACTCCCATGGTGGCGTACCCCCTTTTTTTTTTTTTTTTTTTTT |
| ON9 | RNA11 | TGTTTTGTTTTACCCCTTTGGTTTTTTTTTT |
| ON10 | RNA13 | GGGAGAGGGGTACCCCCTTTTTTTTTTTTTTTTTTTTT |
| ON11 | RNA14 | GGAGGGAGAGGGGTACCCCCTAAACATTTAAATGCAATGAAAA |

TABLE 4
List of plasmids and inserted sequences, used for preparation of
IVT templates. Underlined is the coding sequence from the T7
RNA polymerase transcription start site to the site of linearization.
The recognition sequence for BamHI(GGATCC), PmeI (GTTTAAAC),
AarI (AAAAAAAAAGCAGGTG), Esp3I (AACGTCTC) and Eco52I (CGGCCG)
are in bold with the cut site between nucleotides in italic.

| | |
|---|---|
| PL1 | GCCGAATTATATTATTTTTGCCAAATAATTTTTAACAAAAGCTCTGAAGTCTTCTTCATTTAAATTC |
| | TTAGATGATACTTCATCTGGAAAATTGTCCCAATTAGTAGCATCACGCTGTGTAATACGACTCACT |
| | ATAGGGGGTACGCCACCATGGTGAGCAAGGGCGAGGAGCTGTTCACCGGGGTGGTGCCCATCCTG |
| | GTCGAGCTGGACGGCGACGTAAACGGCCACAAGTTCAGCGTGTCCGGCGAGGGCGAGGGCGATGC |
| | CACCTACGGCAAGCTGACCCTGAAGTTCATCTGCACCACCGGCAAGCTGCCCGTGCCCTGGCCCAC |
| | CCTCGTGACCACCCTGACCTACGGCGTGCAGTGCTTCAGCCGCTACCCCGACCACATGAAGCAGCA |
| | CGACTTCTTCAAGTCCGCCATGCCCGAAGGCTACGTCCAGGAGCGCACCATCTTCTTCAAGGACGA |
| | CGGCAACTACAAGACCCGCGCCGAGGTGAAGTTCGAGGGCGACACCCTGGTGAACCGCATCGAGC |
| | TGAAGGGCATCGACTTCAAGGAGGACGGCAACATCCTGGGGCACAAGCTGGAGTACAACTACAAC |
| | AGCCACAACGTCTATATCATGGCCGACAAGCAGAAGAACGGCATCAAGGTGAACTTCAAGATCCG |
| | CCACAACATCGAGGACGGCAGCGTGCAGCTCGCCGACCACTACCAGCAGAACACCCCCATCGGCG |
| | ACGGCCCCGTGCTGCTGCCCGACAACCACTACCTGAGCACCCAGTCCGCCCTGAGCAAAGACCCC |
| | AACGAGAAGCGCGATCACATGGTCCTGCTGGAGTTCGTGACCGCCGCCGGGATCACTCTCGGCAT |
| | GGACGAGCTGTACAAGTAAGGATCCTAGGGCCGCAGCTCGCTTTCTTGCTGTCCAATTTCTATTAA |
| | AGGTTCCTTTGTTCCCTAAGTCCAACTACTAAACTGGGGGATATTATGAAGGGCCTTGAGCATTTG |
| | GATTCTGCCTAATAAAAAACATTTATTTTCATTGCGTTTAGCTCGCTTTCTTGCTGTCCAATTTCTAT |
| | TAAAGGTTCCTTTGTTCCCTAAGTCCAACTACTAAACTGGGGGATATTATGAAGGGCCTTGAGCAT |
| | TTGGATTCTGCCTAATAAAAAACATTTATTTTCATTGCATTTAAATGTTTAAACAAAAAAAAAAAA |
| | AAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAAAAAAAAAAAGCAGGTGG |
| | GAGGGTACGTTCGTGATAAACAAAC |
| PL2 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATCACGCTGTGTAATAC |
| | GACTCACTATAGGGGGTACGCCACCATGGGAGTCAAAGTTCTGTTTGCCCTGATCTGCATCGCTGT |
| | GGCCGAGGCCAAGCCCACCGAGAACAACGAAGACTTCAACATCGTGGCCGTGGCCAGCAACTTCG |
| | CGACCACGGATCTCGATGCTGACCGCGGGAAGTTGCCCGGCAAGAAGCTGCCGCTGGAGGTGCTC |
| | AAAGAGTTGGAAGCCAATGCCCGGAAAGCTGGCTGCACCAGGGGCTGTCTGATCTGCCTGTCCCA |
| | CATCAAGTGCACGCCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGATTCCTGGGTTCAAG |
| | GACTTGGAGCCCTTGGAGCAGTTCATCGCACAGGTCGATCTGTGTGTGGACTGCACAACTGGCTGC |
| | CTCAAAGGGCTTGCCAACGTGCAGTGTTCTGACCTGCTCAAGAAGTGGCTGCCGCAACGCTGTGCG |
| | ACCTTTGCCAGCAAGATCCAGGGCCAGGTGGACAAGATCAAGGGGGCCGGTGGTGACTAAGGAT |
| | CCTAGGGCCGCAGCTCGCTTTCTTGCTGTCCAATTTCTATTAAAGGTTCCTTTGTTCCCTAAGTCCA |
| | ACTACTAAACTGGGGGATATTATGAAGGGCCTTGAGCATTTGGATTCTGCCTAATAAAAAACATTT |
| | ATTTTCATTGCGTTTAGCTCGCTTTCTTGCTGTCCAATTTCTATTAAAGGTTCCTTTGTTCCCTAAGT |
| | CCAACTACTAAACTGGGGGATATTATGAAGGGCCTTGAGCATTTGGATTCTGCCTAATAAAAAACA |
| | TTTATTTTCATTGCATTTAAATGTTTAAACAAAAAAAAAAAAAAAAAAAAAAAAAAAAAAAAAAA |
| | AAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAGCAGGTGGGAGGGTACGTTCGTGATAAACAAAC |
| PL3 | CTGCAGCCGAATTATATTATTTTTGCCAAATAATTTTTAACAAAAGCTCTGAAGTCTTCTTCATTTA |
| | AATTCTTAGATGATACTTCATCTGGAAAATTGTCCCAATTAGTAGCATGCCACGCTGTGTAATACG |
| | ACTCACTATAGGGATAATCTAGACATTTGCTTCTGACACAACTGTGTTCACTAGCAACCTCAAACA |
| | GACACCATGGGAGTCAAAGTTCTGTTTGCCCTGATCTGCATCGCTGTGGCCGAGGCCAAGCCCACC |
| | GAGAACAACGAAGACTTCAACATCGTGGCCGTGGCCAGCAACTTCGCGACCACGGATCTCGATGC |
| | TGACCGCGGGAAGTTGCCCGGCAAGAAGCTGCCGCTGGAGGTGCTCAAAGAGTTGGAAGCCAATG |
| | CCCGGAAAGCTGGCTGCACCAGGGGCTGTCTGATCTGCCTGTCCCACATCAAGTGCACGCCCAAGA |
| | TGAAGAAGTTCATCCCAGGACGCTGCCACACCTACGAAGGCGACAAAGAGTCCGCACAGGGCGGC |
| | ATAGGCGAGGCGATCGTCGACATTCCTGAGATTCCTGGGTTCAAGGACTTGGAGCCCTTGGAGCAG |
| | TTCATCGCACAGGTCGATCTGTGTGTGGACTGCACAACTGGCTGCCTCAAAGGGCTTGCCAACGTG |
| | CAGTGTTCTGACCTGCTCAAGAAGTGGCTGCCGCAACGCTGTGCGACCTTTGCCAGCAAGATCCAG |
| | GGCCAGGTGGACAAGATCAAGGGGGCCGGTGGTGACTAAGGATCCTAGGGCCGCAGCTCGCTTTC |
| | TTGCTGTCCAATTTCTATTAAAGGTTCCTTTGTTCCCTAAGTCCAACTACTAAACTGGGGGATATTA |
| | TGAAGGGCCTTGAGCATTTGGATTCTGCCTAATAAAAAACATTTATTTTCATTGCGTTTAGCTCGCT |
| | TTCTTGCTGTCCAATTTCTATTAAAGGTTCCTTTGTTCCCTAAGTCCAACTACTAAACTGGGGGATA |
| | TTATGAAGGGCCTTGAGCATTTGGATTCTGCCTAATAAAAAACATTTATTTTCATTGCATTTAAATG |
| | TTTAAACAAAAAAAAAAAAAAAAAAAAAAAAAAAAAAAAAAGCAGGTGGGAGGGTACGTTCGT |
| | GATAAACAAACGGCCGC |
| PL4 | CCTGCAGCCGAATTATATTATTTTTGCCAAATAATTTTTAACAAAAGCTCTGAAGTCTTCTTCATTT |
| | AAATTCTTAGATGATACTTCATCTGGAAAATTGTCCCAATTAGTAGCATGCCACGCTGTGTAATAC |
| | GACTCACTATAGGGGGTACCCCTCTCCCTCCCCCCCTAACGTTACTGGCCGAAGCCGCTTGGAATA |
| | AGGCCGGTGTGCGTTTGTCTATATGTTATTTTCCACCATATTGCCGTCTTTTGGCAATGTGAGGGCC |
| | CGGAAACCTGGCCCTGTCTTCTTGACGAGCATTCCTAGGGGTCTTTCCCCTCTCGCCAAAGGAATG |
| | CAAGGTCTGTTGAATGTCGTGAAGGAAGCAGTTCCTCTGGAAGCTTCTTGAAGACAAACAACGTCT |
| | GTAGCGACCCTTTGCAGGCAGCGGAACCCCCCACCTGGCGACAGGTGCCTCTGCGGCCAAAAGCC |
| | ACGTGTATAAGATACACCTGCAAAGGCGGCACAACCCCAGTGCCACGTTGTGAGTTGGATAGTTGT |
| | GGAAAGAGTCAAATGGCTCTCCTCAAGCGTATTCAACAAGGGGCTGAAGGATGCCCAGAAGGTAC |
| | CCCATTGTATGGGATCTGATCTGGGGCCTCGGTGCACATGCTTTACATGTGTTTAGTCGAGGTTAA |
| | AAAACGTCTAGGCCCCCCGAACCACGGGGACGTGGTTTTCCTTTGAAAAACACGATGATAATATG |
| | GCCACACATATGGGAGTCAAAGTTCTGTTTGCCCTGATCTGCATCGCTGTGGCCGAGGCCAAGCCC |
| | ACCGAGAACAACGAAGACTTCAACATCGTGGCCGTGGCCAGCAACTTCGCGACCACGGATCTCGA |
| | TGCTGACCGCGGGAAGTTGCCCGGCAAGAAGCTGCCGCTGGAGGTGCTCAAAGAGTTGGAAGCCA |
| | ATGCCCGGAAAGCTGGCTGCACCAGGGGCTGTCTGATCTGCCTGTCCCACATCAAGTGCACGCCCA |
| | AGATGAAGAAGTTCATCCCAGGACGCTGCCACACCTACGAAGGCGACAAAGAGTCCGCACAGGGC |
| | GGCATAGGCGAGGCGATCGTCGACATTCCTGAGATTCCTGGGTTCAAGGACTTGGAGCCCTTGGAG |
| | CAGTTCATCGCACAGGTCGATCTGTGTGTGGACTGCACAACTGGCTGCCTCAAAGGGCTTGCCAAC |
| | GTGCAGTGTTCTGACCTGCTCAAGAAGTGGCTGCCGCAACGCTGTGCGACCTTTGCCAGCAAGATC |
| | CAGGGCCAGGTGGACAAGATCAAGGGGGCCGGTGGTGACTAAGGATCCTAGGGCCGCAGCTCGC |
| | TTTCTTGCTGTCCAATTTCTATTAAAGGTTCCTTTGTTCCCTAAGTCCAACTACTAAACTGGGGGAT |
| | ATTATGAAGGGCCTTGAGCATTTGGATTCTGCCTAATAAAAAACATTTATTTTCATTGCGTTTAGCT |
| | CGCTTTCTTGCTGTCCAATTTCTATTAAAGGTTCCTTTGTTCCCTAAGTCCAACTACTAAACTGGGG |
| | GATATTATGAAGGGCCTTGAGCATTTGGATTCTGCCTAATAAAAAACATTTATTTTCATTGCATTTA |
| | AATGTTTAAACAAAAAAAAAAAAAAAAAAAAAAAAAAAAAAAAAAAGCAGGTGGGAGGGTACGT |
| | TCGTGATAAACAAACGGCCGCT |
| PL5 | CCTGCAGCCGAATTATATTATTTTTGCCAAATAATTTTTAACAAAAGCTCTGAAGTCTTCTTCATTT |
| | AAATTCTTAGATGATACTTCATCTGGAAAATTGTCCCAATTAGTAGCATGCCACGCTGTGTAATAC |
| | GACTCACTATAGGGGGTAATGAGTCCTGGACTGAAACGGACTCATATGGGAGTCAAAGTTCTGTTT |
| | GCCCTGATCTGCATCGCTGTGGCCGAGGCCAAGCCCACCGAGAACAACGAAGACTTCAACATCGT |
| | GGCCGTGGCCAGCAACTTCGCGACCACGGATCTCGATGCTGACCGCGGGAAGTTGCCCGGCAAGA |
| | AGCTGCCGCTGGAGGTGCTCAAAGAGTTGGAAGCCAATGCCCGGAAAGCTGGCTGCACCAGGGGC |
| | TGTCTGATCTGCCTGTCCCACATCAAGTGCACGCCCAAGATGAAGAAGTTCATCCCAGGACGCTGC |
| | CACACCTACGAAGGCGACAAAGAGTCCGCACAGGGCGGCATAGGCGAGGCGATCGTCGACATTCC |
| | TGAGATTCCTGGGTTCAAGGACTTGGAGCCCTTGGAGCAGTTCATCGCACAGGTCGATCTGTGTGT |
| | GGACTGCACAACTGGCTGCCTCAAAGGGCTTGCCAACGTGCAGTGTTCTGACCTGCTCAAGAAGTG |
| | GCTGCCGCAACGCTGTGCGACCTTTGCCAGCAAGATCCAGGGCCAGGTGGACAAGATCAAGGGGG |
| | CCGGTGGTGACTAAGGATCCTAGGGCCGCAGCTCGCTTTCTTGCTGTCCAATTTCTATTAAAGGTT |
| | CCTTTGTTCCCTAAGTCCAACTACTAAACTGGGGGATATTATGAAGGGCCTTGAGCATTTGGATTCT |
| | GCCTAATAAAAAACATTTATTTTCATTGCGTTTAGCTCGCTTTCTTGCTGTCCAATTTCTATTAAAG |
| | GTTCCTTTGTTCCCTAAGTCCAACTACTAAACTGGGGGATATTATGAAGGGCCTTGAGCATTTGGA |
| | TTCTGCCTAATAAAAAACATTTATTTTCATTGCATTTAAATGTTTAAACAAAAAAAAAAAAAAAAA |
| | AAAAAAAAAAAAAAAAAGCAGGTGGGAGGGTACGTTCGTGATAAACAAACGGCCGCT |
| PL6 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGGTAGGCGCGCCAGTCCTTCTGTAGCGACCCTTTGCAGGCAGCGGAACCC |
| | CCCACCTGGCGACAGGTGCCTCTGCGGCCAAAAGCCACGTGTATAAGATACACCTGCAAAGGCGG |
| | CACAACCCCAGTGCCACGTTGTGAGTTGGATAGTTGTGGAAAGAGTCAAATGGCTCTCCTCAAGCG |
| | TATTCAACAAGCGACTCGGACATATGGGAGTCAAAGTTCTGTTTGCCCTGATCTGCATCGCTGTGG |
| | CCGAGGCCAAGCCCACCGAGAACAACGAAGACTTCAACATCGTGGCCGTGGCCAGCAACTTCGCG |
| | ACCACGGATCTCGATGCTGACCGCGGGAAGTTGCCCGGCAAGAAGCTGCCGCTGGAGGTGCTCAA |
| | AGAGTTGGAAGCCAATGCCCGGAAAGCTGGCTGCACCAGGGGCTGTCTGATCTGCCTGTCCCACAT |
| | CAAGTGCACGCCCAAGATGAAGAAGTTCATCCCAGGACGCTGCCACACCTACGAAGGCGACAAAG |
| | AGTCCGCACAGGGCGGCATAGGCGAGGCGATCGTCGACATTCCTGAGATTCCTGGGTTCAAGGAC |
| | TTGGAGCCCTTGGAGCAGTTCATCGCACAGGTCGATCTGTGTGTGGACTGCACAACTGGCTGCCTC |
| | AAAGGGCTTGCCAACGTGCAGTGTTCTGACCTGCTCAAGAAGTGGCTGCCGCAACGCTGTGCGACC |
| | TTTGCCAGCAAGATCCAGGGCCAGGTGGACAAGATCAAGGGGGCCGGTGGTGACTAAGGATCCTA |
| | GGGCCGCAGCTCGCTTTCTTGCTGTCCAATTTCTATTAAAGGTTCCTTTGTTCCCTAAGTCCAACTA |
| | CTAAACTGGGGGATATTATGAAGGGCCTTGAGCATTTGGATTCTGCCTAATAAAAAACATTTATTT |
| | TCATTGCGTTTAGCTCGCTTTCTTGCTGTCCAATTTCTATTAAAGGTTCCTTTGTTCCCTAAGTCCAA |
| | CTACTAAACTGGGGGATATTATGAAGGGCCTTGAGCATTTGGATTCTGCCTAATAAAAAACATTTA |
| | TTTTCATTGCATTTAAATGTTTAAACAAAAAAAAAAAAAAAAAAAAAAAAAAAAAAAAAAGCAG |
| | GTGGGAGGGTACGTTCGTGATAAACAAACGGCCGCT |
| PL7 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGGTACCCCTCTCCCTCCCCCCCTAACGTTACTGGCCGAAGCCGCTTGGAA |
| | TAAGGCCGGTGTGCGTTTGTCTATATGTTATTTTCCACCATATTGCCGTCTTTTGGCAATGTGAGGG |
| | CCCGGAAACCTGGCCCTGTCTTCTTGACGAGCATTCCTAGGGGTCTTTCCCCTCTCGCCAAAGGAA |
| | TGCAAGGTCTGTTGAATGTCGTGAAGGAAGCAGTTCCTCTGGAAGCTTCTTGAAGACAAACAACGT |
| | CTGTAGCGACCCTTTGCAGGCAGCGGAACCCCCCACCTGGCGACAGGTGCCTCTGCGGCCAAAAG |
| | CCACGTGTATAAGATACACCTGCAAAGGCGGCACAACCCCAGTGCCACGTTGTGAGTTGGATAGTT |
| | GTGGAAAGAGTCAAATGGCTCTCCTCAAGCGTATTCAACAAGGGGCTGAAGGATGCCCAGAAGGT |
| | ACCCCATTGTATGGGATCTGATCTGGGGCCTCGGTGCACATGCTTTACATGTGTTTAGTCGAGGTTA |
| | AAAAACGTCTAGGCCCCCCGAACCACGGGGACGTGGTTTTCCTTTGAAAAACACGATGATAATAT |
| | GGCCACACATATGGGAGTCAAAGTTCTGTTTGCCCTGATCTGCATCGCTGTGGCCGAGGCCAAGCC |
| | CACCGAGAACAACGAAGACTTCAACATCGTGGCCGTGGCCAGCAACTTCGCGACCACGGATCTCG |
| | ATGCTGACCGCGGGAAGTTGCCCGGCAAGAAGCTGCCGCTGGAGGTGCTCAAAGAGTTGGAAGCC |
| | AATGCCCGGAAAGCTGGCTGCACCAGGGGCTGTCTGATCTGCCTGTCCCACATCAAGTGCACGCCC |
| | AAGATGAAGAAGTTCATCCCAGGACGCTGCCACACCTACGAAGGCGACAAAGAGTCCGCACAGGG |
| | CGGCATAGGCGAGGCGATCGTCGACATTCCTGAGATTCCTGGGTTCAAGGACTTGGAGCCCTTGGA |
| | GCAGTTCATCGCACAGGTCGATCTGTGTGTGGACTGCACAACTGGCTGCCTCAAAGGGCTTGCCAA |
| | CGTGCAGTGTTCTGACCTGCTCAAGAAGTGGCTGCCGCAACGCTGTGCGACCTTTGCCAGCAAGAT |
| | CCAGGGCCAGGTGGACAAGATCAAGGGGGCCGGTGGTGACTAAGGATCCTAGGGCCGCAGCTCG |
| | CTTTCTTGCTGTCCAATTTCTATTAAAGGTTCCTTTGTTCCCTAAGTCCAACTACTAAACTGGGGGA |
| | TATTATGAAGGGCCTTGAGCATTTGGATTCTGCCTAATAAAAAACATTTATTTTCATTGCGTTTAGC |
| | TCGCTTTCTTGCTGTCCAATTTCTATTAAAGGTTCCTTTGTTCCCTAAGTCCAACTACTAAACTGGG |
| | GGATATTATGAAGGGCCTTGAGCATTTGGATTCTGCCTAATAAAAAACATTTATTTTCATTGCATTT |
| | AAATGTTTAAACAAAAAAAAAAAAAAAAAAAAAAAAAAAAAAAAAAAGAGACGGGGTACGTTCGTG |
| | ATACGGCCGCTTCGAGCAGA |
| PL8 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATCACGCTGTGTAATAC |
| | GACTCACTATAGGGGGTACGCCACCATGGGAGTCAAAGTTCTGTTTGCCCTGATCTGCATCGCTGT |
| | GGCCGAGGCCAAGCCCACCGAGAACAACGAAGACTTCAACATCGTGGCCGTGGCCAGCAACTTCG |
| | CGACCACGGATCTCGATGCTGACCGCGGGAAGTTGCCCGGCAAGAAGCTGCCGCTGGAGGTGCTC |
| | AAAGAGTTGGAAGCCAATGCCCGGAAAGCTGGCTGCACCAGGGGCTGTCTGATCTGCCTGTCCCA |
| | CATCAAGTGCACGCCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGATTCCTGGGTTCAAG |
| | GACTTGGAGCCCTTGGAGCAGTTCATCGCACAGGTCGATCTGTGTGTGGACTGCACAACTGGCTGC |
| | CTCAAAGGGCTTGCCAACGTGCAGTGTTCTGACCTGCTCAAGAAGTGGCTGCCGCAACGCTGTGCG |
| | ACCTTTGCCAGCAAGATCCAGGGCCAGGTGGACAAGATCAAGGGGGCCGGTGGTGACTAAGGATC |
| | CTAGGGCCGCAGCTCGCTTTCTTGCTGTCCAATTTCTATTAAAGGTTCCTTTGTTCCCTAAGTCCAA |
| | CTACTAAACTGGGGGATATTATGAAGGGCCTTGAGCATTTGGATTCTGCCTAATAAAAAACATTTA |
| | TTTTCATTGCGTTTAGCTCGCTTTCTTGCTGTCCAATTTCTATTAAAGGTTCCTTTGTTCCCTAAGTC |
| | CAACTACTAAACTGGGGGATATTATGAAGGGCCTTGAGCATTTGGATTCTGCCTAATAAAAAACAT |
| | TTATTTTCATTGCATTTAAATGTTTAAACAAAAAAAAAAAAAAAAAAAAAAAAAAAAAAAAAAG |
| | CAGGTGGGAGGGTACGTTCGTGATAAACAAACGGCCGCT |
| PL9 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGTAAAACAAAACACACCCCTCTCCCTCCCCCCCTAACGTTACTGGCCGAA |
| | GCCGCTTGGAATAAGGCCGGTGTGCGTTTGTCTATATGTTATTTTCCACCATATTGCCGTCTTTTGG |
| | CAATGTGAGGGCCCGGAAACCTGGCCCTGTCTTCTTGACGAGCATTCCTAGGGGTCTTTCCCCTCTC |
| | GCCAAAGGAATGCAAGGTCTGTTGAATGTCGTGAAGGAAGCAGTTCCTCTGGAAGCTTCTTGAAG |
| | ACAAACAACGTCTGTAGCGACCCTTTGCAGGCAGCGGAACCCCCCACCTGGCGACAGGTGCCTCT |
| | GCGGCCAAAAGCCACGTGTATAAGATACACCTGCAAAGGCGGCACAACCCCAGTGCCACGTTGTG |
| | AGTTGGATAGTTGTGGAAAGAGTCAAATGGCTCTCCTCAAGCGTATTCAACAAGGGGCTGAAGGA |
| | TGCCCAGAAGGTACCCCATTGTATGGGATCTGATCTGGGGCCTCGGTGCACATGCTTTACATGTGT |
| | TTAGTCGAGGTTAAAAAACGTCTAGGCCCCCCGAACCACGGGGACGTGGTTTTCCTTTGAAAAACA |
| | CGATGATAATATGGCCACACATATGGTGTCTAAGGGGGAGGAGCTGTTCACCGGCGTGGTGCCCA |
| | TCCTGGTGGAGCTGGACGGCGACGTGAACGGCCACAAGTTCAGCGTGAGCGGGGAGGGCGAGGG |
| | CGACGCCACCTACGGCAAGCTGACCCTGAAGTTCATCTGCACAACGGGGAAGCTGCCCGTGCCCT |
| | GGCCCACCCTGGTGACCACCCTGACCTACGGCGTGCAGTGCTTCAGCCGGTACCCCGACCACATGA |
| | AGCAGCACGACTTCTTCAAGAGCGCCATGCCCGAGGGGTACGTGCAGGAGCGGACCATCTTCTTC |
| | AAGGACGACGGGAACTACAAGACCCGCGCCGAGGTGAAGTTCGAGGGGGACACCCTGGTGAACC |
| | GCATCGAGCTGAAGGGCATCGACTTCAAGGAGGACGGCAACATCCTGGGGCACAAGCTGGAGTAC |
| | AACTACAACAGCCACAACGTGTACATCATGGCCGACAAGCAGAAGAACGGCATCAAGGTGAACTT |
| | CAAGATCCGGCACAACATCGAGGACGGCAGCGTGCAGCTGGCCGACCACTACCAGCAGAACACCC |
| | CCATCGGCGACGGCCCCGTGCTCCTGCCCGACAACCACTACCTGTCCACCCAGAGCGCCCTGAGCA |
| | AGGACCCAAACGAGAAGCGGGACCACATGGTCCTGCTGGAGTTCGTGACCGCCGCCGGCATCACC |
| | CTGGGCATGGACGAGCTGTACAAGCGCTCCCGCGACATCAGCCACGGCTTCCCTCCCGCCGTGGCC |
| | GCCCAGGACGACGGCACCCTGCCCATGAGCTGCGCCCAGGAGAGCGGCATGGACCGGCACCCGGC |
| | CGCCTGCGCCAGCGCCCGGATCAACGTGTGATAGGGATCCAACAAAAAAAACAAAAAAAAAACC |
| | AAAGAGACGCGGCCGCTTCGAGCAGACATGATAAGATACATT |
| PL10 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGGAGACCCTCGACCGTCGATTGTCCACTGGTCAACAATAGATGACTTACA |
| | ACTAATCGGAAGGTGCAGAGACTCGACGGGAGCTACCCTAACGTCAAGACGAGGGTAAAGAGAG |
| | AGTCCAATTCTCAAAGCCAATAGGCAGTAGCGAAAGCTGCAAGAGAATGAAAATCCGTTGACCTT |
| | AAACGGTCGTGTGGGTTCAAGTCCCTCCACCCCCACGCCGGAAACGCAATAGCCGAAAAACAAAA |
| | AACAAAAAAACCCCCCTCTCCCTCCCCCCCTAACGTTACTGGCCGAAGCCGCTTGGAATAAGGCCG |
| | GTGTGCGTTTGTCTATATGTTATTTTCCACCATATTGCCGTCTTTTGGCAATGTGAGGGCCCGGAAA |
| | CCTGGCCCTGTCTTCTTGACGAGCATTCCTAGGGGTCTTTCCCCTCTCGCCAAAGGAATGCAAGGTC |
| | TGTTGAATGTCGTGAAGGAAGCAGTTCCTCTGGAAGCTTCTTGAAGACAAACAACGTCTGTAGCGA |
| | CCCTTTGCAGGCAGCGGAACCCCCCACCTGGCGACAGGTGCCTCTGCGGCCAAAAGCCACGTGTAT |
| | AAGATACACCTGCAAAGGCGGCACAACCCCAGTGCCACGTTGTGAGTTGGATAGTTGTGGAAAGA |
| | GTCAAATGGCTCTCCTCAAGCGTATTCAACAAGGGGCTGAAGGATGCCCAGAAGGTACCCCATTGT |
| | ATGGGATCTGATCTGGGGCCTCGGTGCACATGCTTTACATGTGTTTAGTCGAGGTTAAAAAACGTC |
| | TAGGCCCCCCGAACCACGGGGACGTGGTTTTCCTTTGAAAAACACGATGATAATATGGCCACACAT |
| | ATGGGAGTCAAAGTTCTGTTTGCCCTGATCTGCATCGCTGTGGCCGAGGCCAAGCCCACCGAGAAC |
| | AACGAAGACTTCAACATCGTGGCCGTGGCCAGCAACTTCGCGACCACGGATCTCGATGCTGACCG |
| | CGGGAAGTTGCCCGGCAAGAAGCTGCCGCTGGAGGTGCTCAAAGAGTTGGAAGCCAATGCCCGGA |
| | AAGCTGGCTGCACCAGGGGCTGTCTGATCTGCCTGTCCCACATCAAGTGCACGCCCAAGATGAAG |
| | AAGTTCATCCCAGGACGCTGCCACACCTACGAAGGCGACAAAGAGTCCGCACAGGGCGGCATAGG |
| | CGAGGCGATCGTCGACATTCCTGAGATTCCTGGGTTCAAGGACTTGGAGCCCTTGGAGCAGTTCAT |
| | CGCACAGGTCGATCTGTGTGTGGACTGCACAACTGGCTGCCTCAAAGGGCTTGCCAACGTGCAGTG |
| | TTCTGACCTGCTCAAGAAGTGGCTGCCGCAACGCTGTGCGACCTTTGCCAGCAAGATCCAGGGCCA |
| | GGTGGACAAGATCAAGGGGGCCGGTGGTGACTAAAAAAAACAAAAAACAAAACGGCTATTATGC |
| | GTTACCGGCGAGACGCTACGGACTTAAATAATTGAGCCTTAAAGAAGAAATTCTTTAAGTGGATGC |
| | TCTCAAACTCAGGGAAACCTAAATCTAGTTATAGACAAGGCAATCCTGAGCCAAGCCGAAGTAGT |
| | AATTAGTAAGACCAGTGGACAATCGACGGATAACAGCATATCTAGCGGCCGCTTCGAGCAGACAT |
| | GATAAGATACATTGATGATC |

### EXAMPLES

The following examples are provided solely to demonstrate the invention and to explain its aspects. The following examples should not be considered as limiting or demonstrating the full scope of the invention. Example 1 demonstrates the synthesis of representative transcription initiator containing EDA group suitable for the synthesis of pre-circRNAs-starting material for the circularization according to the chemical circularization method w/o the cap (used as a reference). Examples 2-15 demonstrate the synthesis of a representative transcription initiators containing cap structure equipped with an EDA group suitable for the synthesis of capped pre-circRNAs-starting material for the circularization according to the chemical circularization method that produces capped circRNA. Examples 16-22 describe the preparation of circRNAs according to the invention as well as reference circRNAs obtained by state-of-the-art methods used in comparative studies. Examples 23-25 describe the procedures for circRNA purification and structure confirmation. Example 26 described the evaluation of the translational activity of capped and unmodified circ-mRNAs in a cell-free system. Examples 27 and 28 describe the optimization of RNA conformation for optimal chemical circularization. Example 29 describes the evaluation of the translational activity of circ-mRNAs in living cells.

### Example 1. Synthesis of EDA-PEG₅-AG

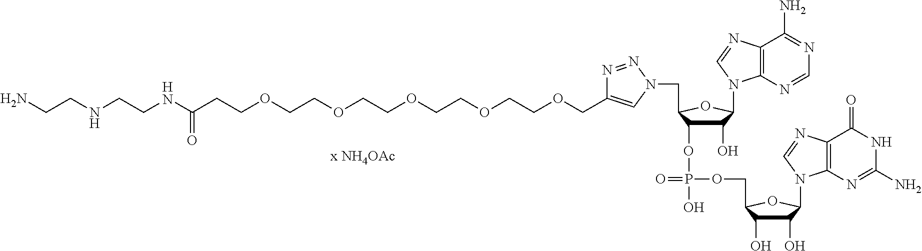

Diethylenetriamine (33.7 μl, 310 μmol, Sigma) was added to a solution of alkyne-PEG5-NHS ester (12.5 mg, 31.2 μmol, Sigma) in DMF (200 μl). After 150 min of vortexing at 20° C. aqueous solution of ammonium acetate (50 μl, 500 mM, HPLC-grade) and acetic acid (60 μl, 50%) were added to afford a mixture of pH ~7. Next, a solution of N3-AG (28 μmol, 2.00 ml) 31 and a solution of Cu(II)-THPTA complex (60 μl, 50 mM) were combined with the mixture. After that, sodium ascorbate (31 mg, 157 μmol) was added, and the reaction mixture was stirred at 20° C. and monitored by HPLC. After two hours solution of triethylammonium acetate (200 μl, 1 M, HPLC-grade), a solution of Cu(II)-THPTA complex (20 μl, 50 mM), and sodium ascorbate (114 mg, 586 μmol) were added, and the reaction was continued overnight. The reaction was quenched with EDTA and resolved using HPLC-column: Gemini 5 μm NX—C18 110 Å 250×10 mm; flow 5 ml/min at 23° C.; solvent A: 50 mM NH₄OAc pH 5.9; solvent B: acetonitrile; elution: from 0% to 41% B in 25 min, then from 41% to 100% B in 10 min, then 100% B for 5 min. HPLC eluate containing the desired product was freeze-dried and resuspended in water in three repetitions to remove the traces of solvents and ammonium acetate. The final product EDA-PEG₅-AG was obtained in the form of a solution of an ion pair with ammonium acetate (9.2 μmol, 95% purity by HPLC) with 33% yield. MS ESI(−): m/z=1025.8, calculated for C₃₈H₅₈N₁₆O₁₆P⁻ m/z: 1025.4.

### Example 2. Synthesis of N3-GDP

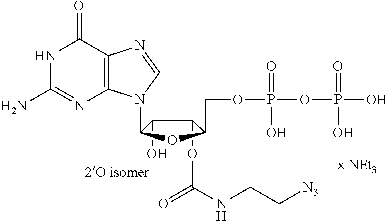

1,1-Carbonyldiimidazole (CDI) (1.21 g, 7.5 mmol, 10 eqiuv.) was added to a solution of GDP (9052.5 mOD₂₆₀, 500 mg, 750 μmol) in DMSO (15 mL). The reaction mixture was stirred at room temperature and monitored by RP-HPLC. After five hours of reaction, CDI excess was hydrolysed with water (200 μL), and the 2-azidoethylamine (7.5 mmol, 620 μL, 10 equiv.) was added, followed by DBU (3.0 mmol, 448 μL). The resulting mixture was stirred overnight at room temperature. The reaction progress was monitored by RP-HPLC. After completion of the linker attachment, the solution wad was diluted with hydrochloric acid (1% aq.) until pH 1 was reached, and the mixture was stirred overnight at room temp. The neutralised mixture was purified on DEAE-Sephadex column. The eluate was concentrated to yield a triethylammonium salt of the final product as a mixture of 2′-O/3′-O regioisomers (7638 mOD₂₆₀, 633 μmol, 84%).

### Example 3. Synthesis of N₃-m⁷GDP

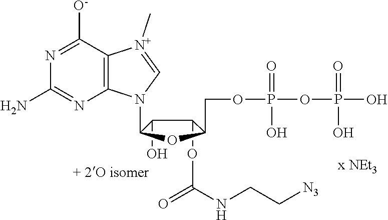

Methyl iodide (6.33 mmol, 394 μL, 10 equiv.) was added to a suspension of N3-GDP (mixture of isomers) (7638 mOD₂₆₀, 350 mg, 633 μmol) in DMSO (6.33 mL), and the resulting mixture was stirred at room temperature. The reaction progress was monitored by RP-HPLC. After four hours the reaction was quenched with water (60 mL) and extracted with diethyl ether (2×50 mL). The aqueous phase was neutralised with NaHCO₃ and purified on a DEAE-Sephadex column. The eluate was concentrated to yield a triethylammonium salt of the final product as a mixture of 2′-O/3′-O regioisomers (5282.6 mOD₂₆₀, 463 μmol, 82%).

### Example 4. Synthesis of N₃-m⁷GpppAmG (or N₃-Cap1)

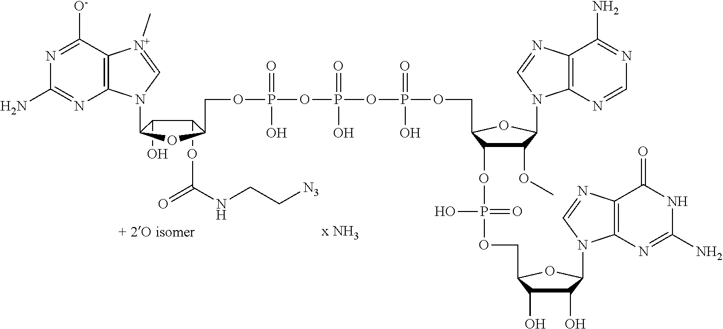

N₃-m⁷GDP (mix of regioisomers) (1778 mOD₂₆₀, 156 μmol, 88 mg, 1.5 equiv) and Im-pAmpG (2833.6 mOD₂₆₀, 78.5 mg, 104 μmol) 21 were suspended in DMF (2.0 mL) and ZnCl₂ (228 mg, 1.67 mmol, 16 equiv.) was added. The resulting mixture was stirred, and the reaction progress was monitored by RP-HPLC. After completion, the reaction was terminated by the addition of aqueous EDTA (20 mL), and the pH was adjusted to 6 with solid NaHCO₃. The product was purified by semipreparative RP-HPLC with the use of a linear gradient (0-40%) of acetonitrile in ammonium acetate (pH 5.9, 0.05 M) over 60 min. Collected fractions were combined and concentrated to remove the excess of ACN. The product (mixture of 2′-O/3′-O regioisomers) (3232 mOD₂₆₀, 55 μmol, 53%) was obtained as a white powder after lyophilisation. ¹H NMR (500 MHz, D₂O, 25° C.): δ=9.21 (s, 1H, H8_{m7G}, isomer 3′), 9.17 (s, 1H, H8_{m7G}, isomer 2′) 8.57 (s, 2H, H8_{A}, isomer 2′ and 3′), 8.31 (s, 2H, H2_{A}, isomer 2′ and 3′), 8.04 (s, 2H, H8_{G}, isomer 2′ and 3′), 6.10 (m, 2H, H1′_{A}, isomer 2′ and 3′), 6.02 (d, ³J_{H-H}=3.3 Hz, 1H, H1′_{m7G} isomer 2′), 5.89 (d, ³J_{H-H}=5.6 Hz, 1H, H1′_{m7G} isomer 3′), 5.80 (m, 2H, H1′_{G}, isomer 2′ and 3′), 5.47 (m, 1H, H2′_{m7G} isomer 2′), 5.23 (m, 1H, H3′_{m7G} isomer 3′), 4.93 (m, 2H, H3′_{A}, isomer 2′ and 3′), 4.78 (m, overlapped with HDO, 2H, H2′_{G}, H2′_{m7G} isomer 3′), 4.67 (m, 1H, H3′_{m7G} isomer 2′), 4.54-4.46 (m, 2H, H3′_{G}, H4′_{m7G} isomer 3′, 2H, H4′_{A}, isomer 2′ and 3′), 4.45-4.41 (m, 2H, H2′_{A}, isomer 2′ and 3′), 4.35 (m, 2H, H4′_{G}, H4′_{m7G} isomer 2′, 4H, H5′_{m7G}, H5″_{m7G}, isomer 2′ and 3′), 4.30-4.14 (m, 8H, H5′_{G}, H5″_{G}, H5′_{A}, H5″_{A}, isomer 2′ and 3′), 4.04 (s, 6H N7-CH_{3 m7G}, isomer 2′ and 3′), 3.49 (m, 6H, CH₃, 2′-O—CH₃, isomer 2′ and 3′), 3.45-3.30 (m, 8H, CH₂—CH₂, isomer 2′ and 3′).

### Example 5. Synthesis of EDA-PEG₅-m⁷GpppAₘG (or EDA-PEG₅-Cap1)

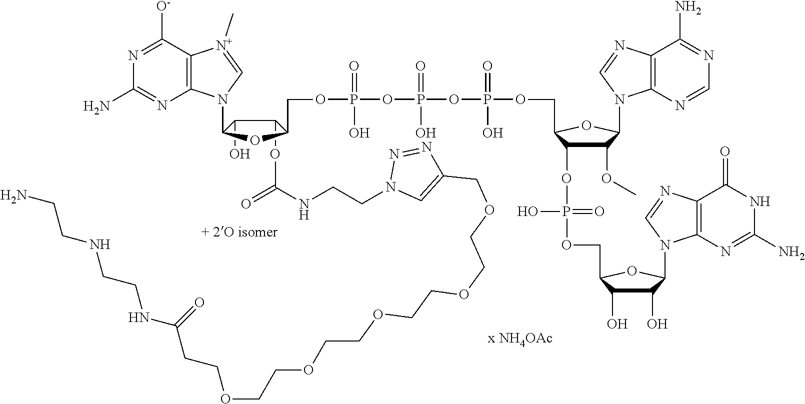

Diethylenetriamine (9.6 μl, 90 μmol, Sigma) was added to a solution of alkyne-PEG₅-NHS ester (12.0 mg, 29.9 μmol, Sigma) in DMF (180 μl). After 60 min of vortexing at 25° C. aqueous solution of triethylammonium acetate (520 μl, 50 mM, HPLC-grade) and acetic acid (62 μl, 50%) were added to afford a mixture of pH 6-7. Next, a solution of N₃-m⁷GpppAₘG (400 μl, 26 μmol) and a solution of Cu(II)-THPTA complex (96 μl, 50 mM) were combined with the mixture. After that, sodium ascorbate (49 mg, 248 μmol) was added, and the reaction mixture was stirred at 25° C. and monitored by HPLC. After two hours a solution of Cu(II)-THPTA complex (48 μl, 50 mM), and sodium ascorbate (25 mg, 126 μmol) were added, and the reaction was continued overnight. The reaction was quenched with EDTA and resolved using HPLC-column: Gemini 5 μm NX—C18 110 Å 250×10 mm; flow 5 ml/min at 23° C.; solvent A: 50 mM NH₄OAc pH 5.9; solvent B: water/acetonitrile mixture 1:2 (v/v); elution: from 0% to 25% B in 45 min, then from 25% to 100% B in 5 min, then 100% B for 5 min. HPLC eluate containing the desired product was freeze-dried and resuspended in water in three repetitions to remove the traces of solvents and ammonium acetate. The final product EDA-PEG₅-AG was obtained in the form of a solution of an ion pair with ammonium acetate (10.5 μmol, 99% purity by HPLC) with 40% yield. HRMS: m/z=1631.42458, 815.20983 (Teor. [M−H]⁻: m/z=1631.42375, [M−2H]²⁻: m/z=815.20824)

### Example 6. Synthesis of 1-(2-tert-butoxycarbamoyl-ethyl)-3-(4′,4″-dimethoxytriphenylmethoxy)-propan-2-ol

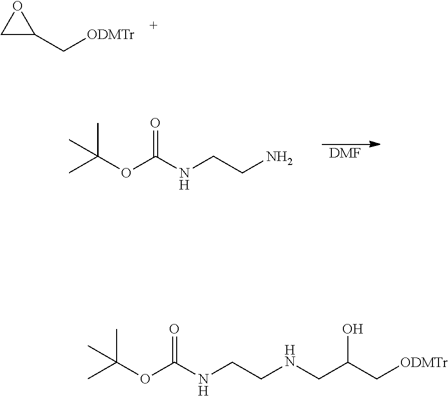

Tert-butyl N-(2-aminoethyl) carbamate (2.35 mL, 14.9 mmol) was added to a solution of DMTr-glycidol (1.12 g, 2.98 mmol; synthesized as described by Punt et al.) in DMF (15 mL) and the mixture was stirred at 60° C. for 24 h. More tert-butyl N-(2-aminoethyl) carbamate (2.35 mL, 14.9 mmol) was added and the mixture was stirred for another 16 h. Then, the DMF was evaporated with toluene and the product was isolated by flash chromatography (silica gel column, 0-10% water in acetonitrile) to give after evaporation 869 mg (1.62 mmol, 54%) of 1-(2-tert-butoxycarbamoyl-ethyl)-3-(4′,4″-dimethoxytriphenylmethoxy)-propan-2-ol as a white gum.

¹H NMR (500 MHz, DMSO-d6, 25° C.): δ=7.40 (m, 2H, ArH), 7.31 (m, 2H, ArH), 7.30 (m, 2H, ArH), 7.22 (m, 4H, ArH), 7.21 (m, 1H, ArH), 6.88 (m, 4H, ArH), 6.69 (t, ³J_{H,H}=5.3 Hz, 1H, —NH-Boc), 4.72 (d, ³J_{H,H}=4.2 Hz, 1H, —OH), 3.73 (s, 6H, CH_{3 DMTr}), 3.68 (m, 1H, H2), 2.97 (m, 2H, Boc-NH—CH₂), 2.93 (m, 1H, H3), 2.86 (dd, ²J_{H,H}=8.9 Hz, ³J_{H,H}=5.9 Hz, 1H, H3′), 2.63 (dd, ²J_{H,H}=11.8 Hz, ³J_{H,H}=4.0 Hz, 1H, H1), 2.52 (m, 2H, Boc-NH—CH₂CH_{2 EDA}), 2.45 (dfd, ²J_{H,H}=11.8 Hz, ³J_{H,H}=7.5 Hz, 1H, H1′z), 1.36 (s, 9H, CH_{3 Boc}) ppm; ¹³C NMR (126 MHz, DMSO-d6, 25° C.): δ=158.0, 155.6, 145.2, 135.9, 135.9, 129.7, 127.8, 127.7, 126.5, 113.1, 85.1, 77.4, 68.9, 66.1, 55.0, 52.7, 49.0, 28.2 ppm

### Example 7. Synthesis of 1-(N,N′-bis(tert-butoxycarbonyl)ethylenediamine)-3-(4′,4″-dimethoxy-triphenylmethoxy)-propan-2-ol

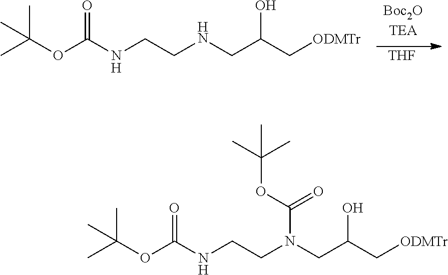

Di-tert-butyl dicarbonate (349 mg, 1.60 mmol) was added to a solution of Boc-EDA-gl-DMTr (859 mg, 1.60 mmol) and TEA (449 μL, 3.20 mmol) in ethyl acetate (4.9 mL) and the mixture was stirred at RT overnight. The product was isolated by flash chromatography (0-100% AcOEt in hexane with 0.5% of TEA) to give after evaporation 955 mg (1.50 mmol, 94%) of 1-(N,N′-bis(tert-butoxycarbonyl)ethylenediamine)-3-(4′,4″-dimethoxytriphenylmethoxy)-propan-2-ol as a white foam. ¹H NMR (500 MHz, DMSO-d6, 25° C.): δ=7.41 (m, 2H, ArH), 7.29 (m, 2H, ArH), 7.26 (m, 4H, ArH), 7.21 (m, 1H, ArH), 6.87 (m, 4H, ArH), 6.77 (m, 1H, —NH-Boc), 4.98 (bs, 1H, —OH), 3.83 (m, 1H, H2), 3.73 (s, 6H, CH_{3 DMTr}), 3.53-2.77 (m, 8H, 4×CH₂), 1.35 (s, 9H, CH_{3 tBu}), 1.34 (s, 9H, CH_{3 tBu}) ppm;

### Synthesis of Boc₂EDA-gl-CEP

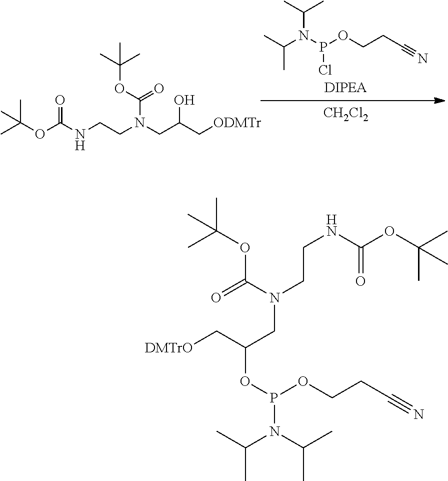

2-Cyanoethyl N,N-diisopropylchlorophosphoramidite (0.665 mL, 2.98 mmol) and DIPEA (1.04 mL, 5.96 mmol) were added to a solution of 1-(3′-N,N′-di-tert-butoxycarbonylethylenediamine)-3-(4′,4″-dimethoxytriphenyl-methoxy) propan-2-ol (949 mg, 1.49 mmol) in dry CH₂Cl₂ (8.3 mL). The mixture was stirred under argon atmosphere for 1 h. Then the reaction mixture was partitioned between AcOEt and 0.1M NaHCO_{3 aq} (100 mL each). The organic fraction was dried over Na₂SO₄ and concentrated in vacuo. The product was isolated by flash chromatography (0-100% AcOEt in hexane with 0.5% (v/v) of TEA) to give after evaporation 937 mg (1.12 mmol, 75%) of phosphoramidite Boc2EDA-gl-CEP (two pairs of diastereomers) as a white foam.³¹P NMR (202 MHz, DMSO-d6, 25° C.): δ=148.8 (s, 1P, P), 148.4 (s, 1P, P), 148.1 (s, 1P, P), 148.0 (s, 1P, P) ppm;

### Example 8. Solid-Phase Synthesis of p-glE-pAₘpG Dinucleotide

2′-O-methyl-N6-benzoyladenosine 3′-O-cyanoethylphosphoramidite (166 mg, 300 μmol) was dissolved in dry acetonitrile (1.0 mL), mixed with 0.3 M BTT in acetonitrile (1.5 mL) and transferred to a 10 mL syringe containing solid-supported guanosine (667 mg of PrimerSupport 5G rG from Cytiva loaded at 300 μmol/g) detritylated previously with 3% dichloroacetic acid in CH₂Cl₂, washed with acetonitrile, and dried in vacuo. The mixture was shaken for 30 min and then the solid support was filtered and washed with acetonitrile. The phosphite was oxidized with 50 mM I2 in pyridine/water (9:1) and detritilated with 3% dichloroacetic acid in CH₂Cl₂. The support was washed with acetonitrile and dried in vacuo. Boc2EDA-gl-CEP (222 mg, 265 μmol) was dissolved in dry acetonitrile (1.0 mL), mixed with 0.3 M BTT in acetonitrile (1.5 mL) and transferred to a 10 ml syringe containing solid-supported AₘpG (200 μmol). The mixture was shaken for 30 min and then the solid support was filtered and washed with acetonitrile. The phosphite was oxidized with 50 mM I2 in pyridine/water (9:1) and detritilated with 3% dichloroacetic acid in CH₂Cl₂. The support was washed with acetonitrile and dried in vacuo. Then, the solid-supported dinucleotide was subjected to phosphitylation using bis(2-cyanoethyl)-N,N-diisopropylphosphoramidite (180 μL, 600 μmol) in acetonitrile (1.0 mL) with 0.3 M BTT (1.5 mL). The product was oxidized with 50 mM I₂ in pyridine/water (9:1) and cleaved from the solid support using AMA (33% ammonium hydroxide/40% methylamine, 1:1 v/v). TBDMS groups were removed using TEA·3HF (250 μL) in DMSO (200 μL) at 65° C. The crude products were isolated by ion-exchange chromatography on DEAE Sephadex and the Boc-protected dinucleotide was separated from pAₘpG by RP HPLC (Grace Reveleris). The Boc groups were removed by heating a solution of protected dinucleotide in 5% acetic acid at 95° C. for 9 h. The product was isolated by ion-exchange chromatography on DEAE Sephadex and purified by RP HPLC to give after evaporation an ammonium salt of p-glE-pAₘpG (28.3 mg, 764 mOD_{260 nm}, 28.2 μmol, purity 95%) as a white solid. The residue was dissolved in 0.6M TEAB (3 mL) and evaporated to dryness with ethanol.

RP-HPLC: Rₜ=5.477 min for D1 isomer and 5.568 min for D2 isomer; HR-MS ESI: m/z calc. for C₂₆H₄₀N₁₂O₁₈P₃⁻ [M−H]⁻: 901.18019, found: 901.18071;

### Example 9. Synthesis of m₂^{7,2′-O}Gppp-glE-pAₘpG (Cap-MW3)

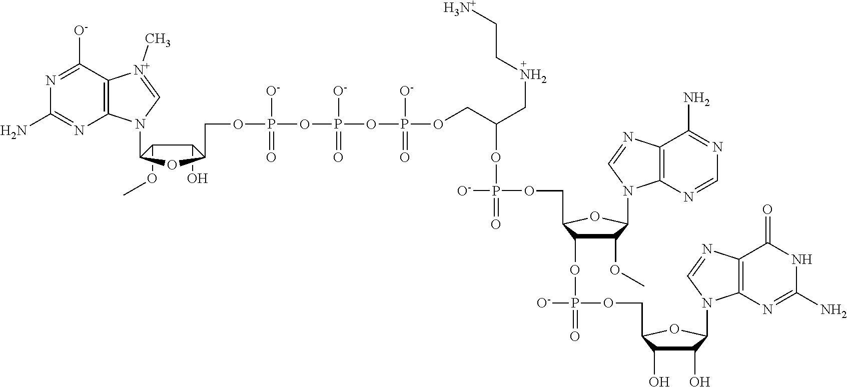

p-glE-pAₘpG (28.3 mg, 764 mOD_{260 nm}, 28.2 μmol), m₂^{7,2′-O}GDP-Im (23.9 mg, 42.3 μmol), and ZnCl₂ (92.0 mg, 677 μmol) were suspended in DMF (565 μmol) and DMSO (565 μmol) mixture and stirred at room temperature for 3 days. The product was isolated by ion-exchange chromatography on DEAE Sephadex and purified by RP HPLC to give after evaporation an ammonium salt of m₂^{7,2-O}Gppp-glE-pAₘpG (6.5 mg, 148 mOD_{260 nm}, 4.63 μmol, 11%) as a white solid. RP-HPLC: Rₜ=7.935 min for D1 isomer and 8.065 min for D2 isomer; HR-MS ESI: m/z calc, for C₃₈H₅₇N₁₇O₂₈P₅⁻ [M−H]⁻: 1354.22525, found: 1354.22542 for D1 isomer and 1354.22669 for D2 isomer.

### Example 10. Synthesis of N6-Propargyl A (Bz)-CE-Phosphoramidite

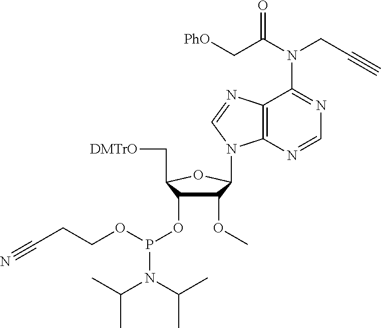

Commercially available 2′-OMe-A (Bz)-CE-Phosphoramidite (500 mg, 540 μmol) was dissolved in 5.5 mL of DCM. To that, 80% wt. solution of propargyl bromide in toluene (233 μL, 4 equiv.) and an aqueous solution of 1 M NaOH with 1 M TBABr (5.5 mL) as a phase transfer catalyst were added. The reaction was stirred vigorously until full conversion of the substrate on TLC was observed. The reaction mixture was extracted with 100 mL of 1:1 (v/v) mixture of diethyl ether and water and the water layer was extracted 3 more times with 50 mL of DCM. The organic layer was dried over anhydrous Na₂SO₄ and evaporated to dryness under reduced pressure. The product was purified by flash column chromatography (0 to 100% AcOEt in hexane+0.5% TEA (v/v)). Final yield: 81% (418 mg, 435 μmol).

### Example 11. Solid-Phase Synthesis of p(N-Propargyl)⁶AₘpG Dinucleotide

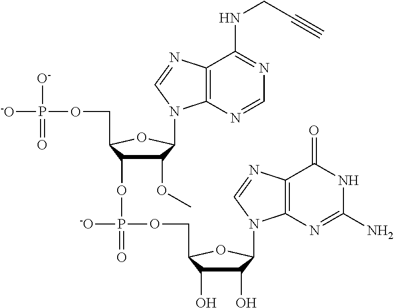

p(N-propargyl)⁶AₘpG dinucleotide dinucleotide was prepared by phosphoramidite approach solid phase synthesis on RiboG solid support (960 mg, 291 μmol, 303 μmol/g loading, 1 equiv.) and compound N6-propargyl A (Bz)-CE-phosphoramidite (418 mg, 437 μmol, 1.5 equiv.). Final yield of the dinucleotide product: 64% (185 μmol, 4995 mOD).

### Example 12. Synthesis of Im-p(N-propargyl)⁶AₘpG

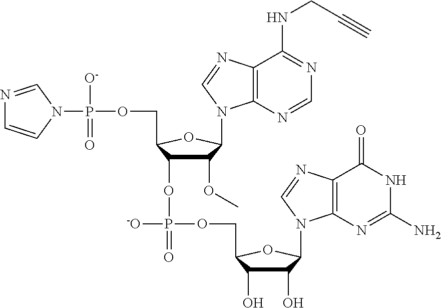

p(N-propargyl)⁶AₘpG dinucleotide (185 μmol, 4995 mOD) was dissolved in 3.7 mL of DMF. To the resulting solution, 2,2′-dithiodipyridine (245 mg, 1110 μmol, 6 equiv.), (202 mg, 2960 μmol, 16 equiv.), TEA (150 μL, 1110 μmol, 6 equiv.) and PPh₃ (291 mg, 1110 μmol, 6 equiv.) were added in that order. The reaction mixture was stirred for 2 h until full conversion of dinucleotide was observed. Then, a solution of NaClO₄ in MeCN (37 mL, 25 mM) was added to precipitate the P-imidazolide sodium salt. The suspension was cooled in the freezer at −20° C. The precipitate was centrifuged and washed with cold MeCN 5 times and dried under vacuum. The final mass of the dried product was 111 mg.

### Example 13. Synthesis of m₂^{2′-O, 7}Gppp (N-propargyl)⁶AₘpG

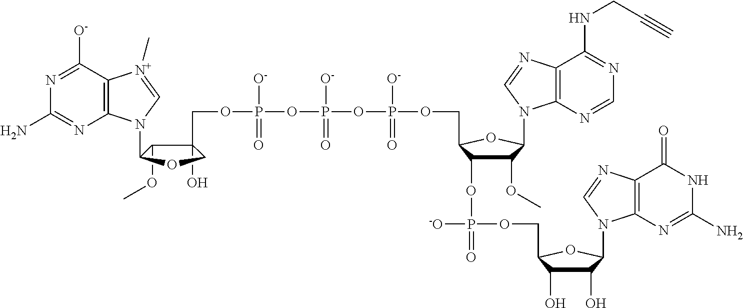

Im-p(N-propargyl)⁶AₘpG (55.7 mg, 93 μmol) was dissolved in 1.2 mL of DMSO then m₂^{2′-O, 7}GDP (121 μmol, 1.3 equiv.) and ZnCl₂ (100 mg, 740 μmol, 8 equiv.) were added. The reaction mixture was stirred at 25° C. and monitored by HPLC. After 16 h full conversion of P-imidazolide was observed. Then, the reaction was quenched with a solution of 20 mg/mL EDTA with 10 mg/mL NaHCO₃ (13.6 mL, 10 equiv. of EDTA). The product was purified by means of ion exchange chromatography on DEAE Sephadex resin (0 to 1.2M TEABaq buffer). The final product (56 μmol) was obtained with 60% yield. MS ESI(−): m/z=597.6, calculated for C₃₆H₄₅N₁₅O₂₄P₄₂⁻ m/z: 598.6.

### Example 14. Synthesis of m₂^{2′-O, 7}Gppp (L1)⁶AₘpG (Cap-AR67)

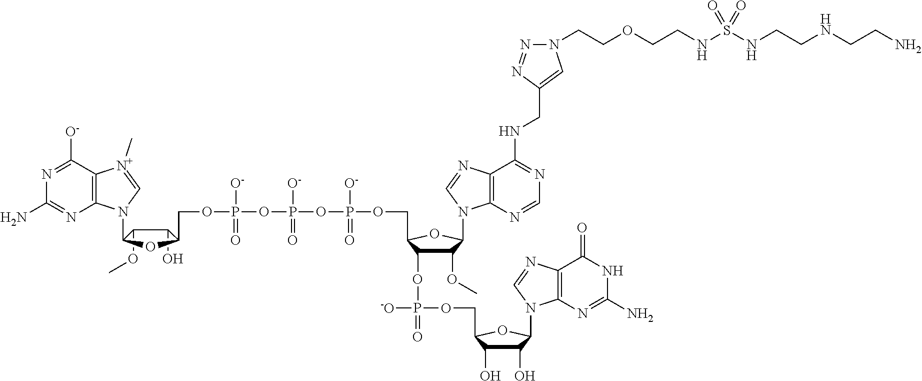

m₂^{2-O, 7}Gppp (N-propargyl)⁶AₘpG (11.6 mg, 320 mOD, 10 μmol) was dissolved in 100 μL of 7:3 (v/v) 1 M Tris pH 7,5 and DMSO mixture. After that azide linker 1 (7.8 mg, 15 μmol, 1.5 equiv.) was added, alongside 50 mM CuSO₄/THPTA (1:1.2) complex solution (100 μL, 5 μmol, 0.5 equiv.) and sodium L-ascorbate (19.8 mg, 100 μmol, 10 equiv.) were added. The reaction mixture was stirred at 25° C. and monitored by HPLC. After 30 minutes full conversion of the substrate was observed and the reaction was quenched with 50 mM EDTA pH 6.5 (200 μL, 10 μmol, 1 equiv.). The final product was separated out of the reaction mixture by preparative RP-HPLC (0% to 30% MeCN in ammonium acetate buffer pH 5.9). The final product (2.2 μmol) was obtained as a white powder after lyophilisation with 22% yield. MS ESI(−): m/z=745.2, calculated for C₄₄H₆₆N₂₂O₂₇P₄S²⁻ m/z: 745.8.

### Example 15. Synthesis of m₂^{2′-O, 7}Gppp (L2)⁶AₘpG (AR-103)

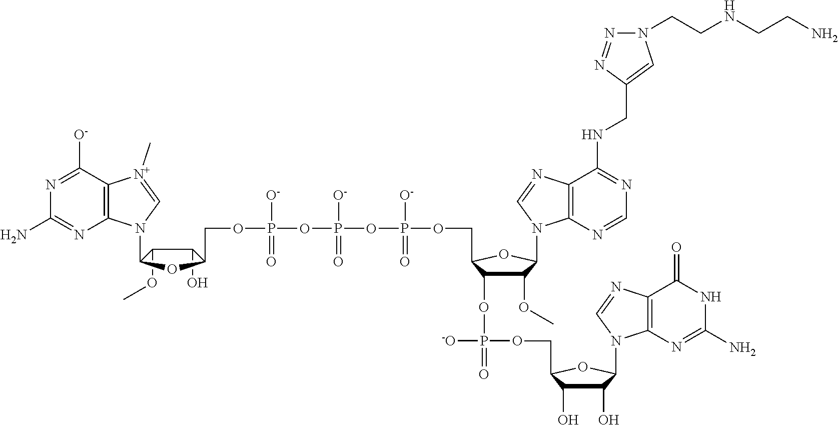

m₂^{2-O, 7}Gppp (N-propargyl)⁶AₘpG (11.6 mg, 320 mOD, 10 μmol) were dissolved in 100 μL of 1:1 (v/v) 1 M Tris pH 7,5 and tert-BuOH mixture. Then azide linker 2 (2.5 mg, 7.5 μmol, 1.5 equiv.) was added, alongside 100 mM CuSO₄/THPTA (1:1.2) complex solution (25 μL, 2.5 μmol, 0.5 equiv.) and sodium L-ascorbate (9.9 mg, 50 μmol, 10 equiv.). The reaction mixture was stirred at 25° C. and monitored by HPLC. After 30 minutes full conversion of the substrate was observed. The reaction was quenched with 50 mM EDTA pH 6.5 (100 μL, 5 μmol, 1 equiv.) and the product was separated out of the reaction mixture by preparative RP-HPLC (0% to 50% MeCN in ammonium acetate buffer pH 5.9). Combined fractions containing pure Boc-protected product were freeze dried. The remaining pellet was dissolved in 1 mL of 100 mM TFA and stirred for 72 h at 30° C. Then, the reaction mixture was diluted with ammonium acetate buffer pH 5.9 to a volume of 5 mL and purified on preparative RP-HPLC (0% to 30% MeCN in ammonium acetate buffer pH 5.9). The final product (0.8 μmol) was obtained as a white powder after lyophilisation with 16% yield. MS ESI(−): m/z=662.1, calculated for C₄₀H₅₆N₂₀O₂₄P₄²⁻ m/z: 661.9.

### Example 16. DNA Template Preparation

The plasmids were prepared by cloning a sequence of choice (Table 4), containing a T7 RNA polymerase promoter and a site for a specific restrictase cleavage, into a pJet vector. To afford the desired DNA template for IVT the plasmids were linearised with a restriction enzyme according to manufacturer's protocol (Thermo). For details see Table 2.

### Example 17. General Protocol for Preparation of EDA-Linker-RNA (Pre-circRNA 2, 4-6, 8, 10, 12, 13, 15, 17)

| | C_{stock} | V_{stock} [μl] | C_{fin} |
|---|---|---|---|
| H₂O | | up to 50 μl | |
| Transcription Buffer x5 | x5 | 10 | x1 |
| U/A/CTP [mM] | 33.3 | 9 | 6 |
| GTP [mM] | 100 | 2 | 4 |
| EDA-linker-AG | 40 | 15 | 12 |
| (or EDA-linker-Cap) [mM] | | | |
| DNA template [ng/μl] | 400 | 5 | 40 |
| MgCl₂ [mM] | 1 000 | 1.25 | 25 |
| Ppase [U/μl] | 0.1 | 1 | 0.002 |
| Ribolock [U/μl] | 40 | 1.25 | 1 |
| T7 RNAP HC [U/μl] | 200 | 5 | 20 |

All ingredients of the reaction mixture listed in the table above were mixed in room temperature in the following order: water, Transcription Buffer ×5 (200 mM Tris-HCl pH 7.9, 30 mM MgCl₂, 50 mM DTT, 50 mM NaCl and 10 mM spermidine, Thermo), NTPs (Thermo), EDA-linker-AG (or EDA-linker-Cap), DNA template, MgCl₂, Ribolock (Thermo), and T7 RNA polymerase HC (Thermo). The reaction mixture was vortexed, gently centrifuged, and incubated for 2 h at 37° C. Next, DNase I (2.0 μl, 1 U/μl, Thermo) was added, and the mixture was incubated for 30 min at 37° C. The RNA product was isolated from the reaction mixture using Monarch RNA cleanup kit (500 μg, NEB). The isolated RNA (~200 μg) was resolved using RP-HPLC—column: PolymerX RP-1 3 μm 100 Å, 4.1×150 mm; flow 1.0 ml/min at 60° C.; solvent A: 100 mM TEAA pH 7.0; solvent B: 200 mM TEAA pH 7.0/acetonitrile 1:1 (v/v); elution: from 20% to 32.5% B in 25 min, then from 32.5% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the RNA product (100-180 μg, 50-90% of the crude product).

### Example 18. General Protocol for Preparation of p-RNA (Pre-circRNA 1, 3, 7, 9, 11, 14, 16)

| | C_{stock} | V_{stock} [μl] | C_{fin} |
|---|---|---|---|
| H₂O | | up to 50 μl | |
| Transcription Buffer x5 | x5 | 10 | x1 |
| U/A/CTP [mM] | 33.3 | 9 | 6 |
| GTP [mM] | 100 | 1.5 | 3 |
| GMP [mM] | 100 | 10 | 20 |
| DNA template [ng/μl] | 400 | 5 | 40 |
| MgCl₂ [mM] | 1 000 | 1.25 | 25 |
| Ppase [U/μl] | 0.1 | 1 | 0.002 |
| Ribolock [U/μl] | 40 | 1.25 | 1 |
| T7 RNAP HC [U/μl] | 200 | 5 | 20 |

All ingredients of the reaction mixture listed in the table above were mixed at 37° C. in the following order: water, Transcription Buffer ×5 (200 mM Tris-HCl pH 7.9, 30 mM MgCl₂, 50 mM DTT, 50 mM NaCl and 10 mM spermidine, Thermo), NTPs (Thermo), GMP (preincubated at 37° C.), DNA template, MgCl₂, Ribolock (Thermo), and T7 RNA polymerase HC (Thermo). The reaction mixture was vortexed, gently centrifuged, and incubated for 2 h at 37° C. Next, DNase I (2.0 μl, 1 U/μl, Thermo) was added, and the mixture was incubated for 30 min at 37° C. The crude RNA product was isolated from the reaction mixture using Monarch RNA cleanup kit (500 μg, NEB). The isolated crude RNA (~150 μg, 2.5-3.5 g of RNA per one litter of IVT reaction mixture) was resolved using RP-HPLC—column: PolymerX RP-1 3 μm 100 Å, 4.1×150 mm; flow 1.0 ml/min at 60° C.; solvent A: 100 mM TEAA pH 7.0; solvent B: 200 mM TEAA pH 7.0/acetonitrile 1:1 (v/v); elution: from 20% to 32.5% B in 25 min, then from 32.5% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the RNA product (70-130 μg, 50-90% of the crude product).

### Example 19. Chemical Circularization of RNA

A diluted solution of the precursor RNA (30-40 μg in 490 μl, ~140 μM, pre-circRNA 2, 4-6, 8, 10, 12, 13, 15, 17) was mixed with buffer (70 μl, 800 mM KH₂PO₄, 200 mM NaCl, pH 7.0) and incubated at 65° C. for 5 min. After that, the hot solution was transferred on bench, where it cooled to room temperature. After 10-15 min at room temperature, a fresh solution of sodium periodate (70 μl, 10 mM) was added, and the reaction mixture was immediately placed in dark and incubated at 25° C. After 30 min, a fresh solution of sodium cyanoborohydride (70 μl, 200 mM) was added, and the incubation was continued for the next 90 min. The crude circRNA product was isolated from the reaction mixture using Monarch RNA cleanup kit (50 μg, NEB). The isolated RNA (80-99% of the initial amount) was resolved using RP-HPLC—column: PolymerX RP-1 3 μm 100 Å, 4.1×150 mm; flow 1.0 ml/min at 60° C.; solvent A: 100 mM HAA pH 7.0; solvent B: 100 mM HAA pH 7 in 75% acetonitrile; elution: from 55% to 60% B in 25 min, then from 60% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (5-10 μg, 15-25% of the initial amount). For additional purification and removal of linear RNA, the product was treated with RNase R as described below (see section RNase R digestion).

### Example 20. Circularization of RNA in the Presence of a Complementary Oligonucleotide

A diluted solution of the precursor RNA (30-40 μg in 480 μl, ~145 μM, pre-circRNA 2, 4-6, 8, 10, 12, 13, 15, 17, 18, 19, 21, 24, 25, 27, 28, 30-32, 34, 35) was mixed with buffer (70 μl, 800 mM KH₂PO₄, 200 mM NaCl, pH 7.0), and complementary oligonucleotide (~11 μl, 10 μM, 1.5 eq, see Table 3 for sequence) and incubated at 65° C. for 5 min. After that, the hot solution was transferred on bench, where it cooled to room temperature. After 10-15 min at room temperature, a fresh solution of sodium periodate (70 μl, 10 mM) was added, and the reaction mixture was immediately placed in the dark and incubated at 25° C. After 30 min, a fresh solution of sodium cyanoborohydride (70 μl, 200 mM) was added, and the incubation was continued for the next 90 min. The crude circRNA product was isolated from the reaction mixture using Monarch RNA cleanup kit (50 μg, NEB). The isolated RNA was resolved using RP-HPLC—column: PolymerX RP-1 3 μm 100 Å, 4.1×150 mm; flow 1.0 ml/min at 60° C.; solvent A: 100 mM HAA pH 7.0; solvent B: 100 mM HAA pH 7 in 75% acetonitrile; elution: from 55% to 60% B in 25 min, then from 60% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (5-10 μg, 15-25% of the initial amount). For additional purification and removal of linear RNA, the product was treated with RNase R as described below (see section RNase R digestion).

### Example 21. Enzymatic Circularization with T4 RNA Ligase I

A diluted solution of the precursor RNA (10-15 μg in 95 μl, ~150 μM, pre-circRNA 1, 3, 7, 9, 11, 14, 16, 20) was mixed with buffer (5 μl, 100 mM Tris-HCl, 500 mM NaCl, pH 7.0) and incubated at 65° C. for 5 min. After that, the hot solution was transferred on bench, where it cooled to room temperature. After 10-15 min at room temperature, T4 RNA ligase 1 buffer ×10 (15 μl, 500 mM Tris-HCl, 100 mM MgCl₂, 10 mM DTT, pH 7.5, NEB), PEG 8000 (15 μl, 50%, NEB), ATP (0.75 μl, 10 mM, NEB), RiboLock RNase inhibitor (3.75 μl, 40 U/μl, Thermo), and T4 RNA ligase 1 (15 μl, 10 U/μl, NEB) were added, and the reaction mixture was incubated at 25° C. for 150 min. The crude circRNA product was isolated from the reaction mixture using Monarch RNA cleanup kit (10 μg, NEB). The isolated RNA (80-99% of the initial amount) was resolved using RP-HPLC—column: RNASept Prep 2 μm 100 Å, 7.8×50 mm; flow 1.0 ml/min at 60° C.; solvent A: 100 mM HAA pH 7.0 in 10% acetonitrile; solvent B: 100 mM HAA pH 7 in 75% acetonitrile; elution: from 20% to 70% B in 25 min, then from 70% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (1-4 μg, 10-30% of the crude product). For additional purification and removal of linear RNA, the product was treated with RNase R as described below (see section RNase R digestion).

### Example 22. Enzymatic Circularization with T4 RNA Ligase II

A diluted solution of the precursor RNA (30-40 μg in 220 μl, ~320 μM, pre-circRNA 1, 3, 7, 9, 11, 14, 18A, 23, 26, 29) was mixed with complementary oligonucleotide (~11 μl, 10 μM, 1.5 eq, see Table 3 for sequence) and incubated at 65° C. for 5 min. After that, the hot solution was transferred on bench, where it cooled to room temperature. After 10-15 min at room temperature, T4 RNA ligase 2 buffer ×10 (35 μl, 500 mM Tris-HCl, 20 mM MgCl₂, 10 mM DTT, 4 mM ATP, pH 7.5, NEB), PEG 8000 (70 μl, 50%, NEB), RiboLock RNase inhibitor (8.75 μl, 40 U/μl, Thermo), and T4 RNA ligase 2 (3.5 μl, 10 U/μl, NEB) were added, and the reaction mixture was incubated at 25° C. for 150 min. The crude circRNA product was isolated from the reaction mixture using Monarch RNA cleanup kit (500 μg, NEB). The isolated RNA (80-99% of the initial amount) was resolved using RP-HPLC—column PolymerX RP-1 3 μm 100 Å, 4.1×150 mm; flow 1.0 ml/min at 60° C.; solvent A: 100 mM HAA pH 7.0; solvent B: 100 mM HAA pH 7 in 75% acetonitrile; elution: from 55% to 60% B in 25 min, then from 60% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (5-10 μg, 15-25% of the initial amount). For additional purification and removal of linear RNA, the product was treated with RNase R as described below (see section RNase R digestion).

### Example 23. RP-HPLC of circRNA

All HPLC separations were performed on modular, low-pressure gradient HPLC apparatus manufactured by Shimadzu (LC-40/LC-20 series) with a thermostated column holder or column oven, DAD detector and fluorescence detector. The mobile phase was composed of non-autoclaved MQ water, HPLC-grade acetonitrile (J. T. Baker), high quality triethylamine (Bioultra, Sigma), HPLC-grade n-hexylamine (Ottokemi), and HPLC-grade glacial acetic acid (J. T. Baker). Solvents of desired composition, concentration and pH were filtered with 0.45 μm filter and stored in the dark at 4° C. for longer periods of time. Chromatograms were recorded with the detection of absorbance at 260 nm (ref 360 nm, 10 nm bandwidth), absorbance spectrum (220-700 nm), and fluorescence of interest (Cy3-550/565, Cy5-650/665).

### Example 24. RNase R Digestion

A solution of RNA (43 μl, 10 μg) was incubated at 65° C. for 5 min and rapidly cooled in an ice bath. After 5 min at 0-4° C., RNase R buffer ×10 (5 μl, 200 mM Tris-HCl, 1 M KCl, 1 mM MgCl₂, pH 7.5, ABM), RiboLock RNase inhibitor (1.25 μl, 40 U/μl, Thermo), and RNase R (1 μl, 10 U/μl, ABM) were added. After 30 min of incubation at 37° C. the products of digestion were analyzed with PAGE directly or isolated from the reaction mixture using Monarch RNA cleanup kit (10 μg, NEB).

### Example 25. RNase H Assay

A solution (8.5 μl) containing circRNA2 (100 ng), DNA oligonucleotide (0 or 150 nM, CCTTCAGCTCGATGCGGTTCA), and RNase H buffer ×1 (20 mM Tris-HCl, 10 mM KCl, 8 mM MgCl₂, 1 mM DTT, Thermo) was heated to 65° C. for 5 min and gradually cooled to 37° C. for 30 min. Next, RiboLock RNase inhibitor (0.5 μl, 40 U/μl, Thermo), and RNase H (1 μl, 5 U/μl, Thermo) were added. After 30 min of incubation at 37° C. the products of digestion were directly analyzed with PAGE.

### Example 26. Translation Inhibition in RRL System

Flexi Rabbit Reticulocyte Lysate (4 μL, Promega) was diluted with 4 μL solution containing amino acid mixture (100 μM), KOAc (500 mM, and MgCl₂ (2.5 mM), and incubated for 1 h at 30° C. Next, 2 μL of RNA (50 ng/μl) mixed with m⁷GpsppG (0-200 μM) was added to 8 μL of the lysate mixture and incubated for 1 h at 30° C. Next, the reaction was immediately placed into a microplate reader (Synergy H1, BioTek). 50 μL of h-coelenterazine (xyz) in 1×PBS was added and the luminescence was measured at 25° C. The assay was repeated to calculate the mean and standard error (SEM).

### Example 27. FRET Probe Synthesis

The Cy5-RNA1-Cy3 FRET probe was synthesized according to the previously reported protocol.31 First in vitro transcription of RNA1 sequence was performed in the presence of N3-AG transcription initiator. The crude product of IVT was isolated by Monarch RNA cleanup kit (NEB) and subjected to 5′-Cy5 and 3′-Cy3 dual labeling. The crude labeling product was isolated from the labeling reaction mixture by Monarch RNA cleanup kit (NEB) and subsequently resolved with HPLC. The HPLC eluate containing the desired product was concentrated by precipitation dried in vacuo and suspended in water to afford the Cy5-RNA1-Cy3 FRET probe solution.

### Example 28. FRET Experiments

The Cy5-RNA1-Cy3 FRET probe solution was diluted with buffer (80 mM KH₂PO₄, 20 mM NaCl, pH 7.0, final volume of 100 μl) to afford a final probe concentration of 100 nM and transferred to a quartz fluorescence cuvette (1×1×350 mm). Optionally, a solution of a complementary DNA oligonucleotide (2 μl, 10 μM, ON2-ON5) was added. The mixture was heated from 20 to 90° C. in 8 min and gradually cooled to 20° C. or 4° C. in 20-30 min. Emission spectra were recorded on a Cary Eclipse spectrofluorometer (Agilent), equipped with a xenon lamp, single cell peltier accessory (spvf 1x0, Agilent), excitation at 500 nm, emission 510-800 nm, 10 mm slit.

### Example 29. Cell Culture and Transfections

The analysis was performed in four cell lines, i.e. HEK 293T, Hep G2, A549, and HeLa, as well as in mouse dendritic cells and macrophages established from bone marrow monocytes. One day before transfection, 8×10³ cells were seeded per well of a 96-well plate. The next day, cells were transfected with RNA. In each cell line, each RNA variant was transfected in triplicate. Transfection was performed with Lipofectamine Messenger MAX (Invitrogen, Waltham, MA, USA), according to the manufacturer's instructions. During transfection, the molar concentration of each RNA was taken into account so that an equal amount of RNA molecules was added to each well. Briefly, for each reaction 0.15 μL of Lipofectamine was first diluted (Mix 1) in 5 μL of Opti-MEM (GIBCO, Grand Island, NY, USA) medium and incubated at room temperature for 10 minutes. In another tube, the RNA was diluted in 5 μL of Opti-MEM medium (Mix 2). Next, both Mix 1 and Mix 2 were mixed and incubated for 5 minutes at room temperature. Afterward, 10 μL of the mixture was transferred to the appropriate well of a 96-well plate containing previously seeded cells. After the transfection, the cells were incubated in standard culture conditions (37° C., 5% CO2, saturating humidity). At appropriate time points, 20 μL of medium from each well was withdrawn to test the amount of produced protein, and the remaining medium was completely replaced with the fresh one. The amount of luciferase released into the medium was analyzed using the GLuc GLOW Assay (NanoLight Technologies, Norman, OK, USA) reagents, according to the manufacturer's instructions. Luminescence was read in white, opaque plates. Measurements were made using an En Vision plate reader (Perkin Elmer, Waltham, MA, USA).

### REFERENCES

(1) Qin, S.; Tang, X.; Chen, Y.; Chen, K.; Fan, N.; Xiao, W.; Zheng, Q.; Li, G.; Teng, Y.; Wu, M.; et al. mRNA-based therapeutics: powerful and versatile tools to combat diseases. Signal Transduction and Targeted Therapy 2022, 7 (1). DOI: 10.1038/s41392-022-01007-w (acccessed 2022-10-07T12:03:06). Janowski, M.; Andrzejewska, A. The legacy of mRNA engineering: A lineup of pioneers for the Nobel Prize. Molecular Therapy—Nucleic Acids 2022, 29, 272-284. DOI: 10.1016/j.omtn.2022.07.003 (acccessed 2022-10-07T11:43:22).
(2) Garneau, N. L.; Wilusz, J.; Wilusz, C. J. The highways and byways of mRNA decay. Nature Reviews Molecular Cell Biology 2007, 8 (2), 113-126. DOI: 10.1038/nrm2104. Houseley, J.; Tollervey, D. The Many Pathways of RNA Degradation. Cell 2009, 136 (4), 763-776. DOI: 10.1016/j.cell.2009.01.019 (acccessed 2022-10-07T12:34:14).
(3) Jeck, W. R.; Sharpless, N. E. Detecting and characterizing circular RNAs. Nature Biotechnology 2014, 32 (5), 453-461. DOI: 10.1038/nbt.2890 (acccessed 2022-10-07T12:45:35).
(4) Dolinnaya, N. G.; Blumenfeld, M.; Merenkova, I. N.; Oretskaya, T. S.; Krynetskaya, N.; Ivanovskaya, M. G.; Vasseur, M.; Shabarova, Z. A. Oligonucleotide circularization by template-directed chemical ligation. Nucleic Acids Research 1993, 21 (23), 5403-5407. DOI: 10.1093/nar/21.23.5403 (acccessed Oct. 7, 2022). Fedorova, O. A.; Gottikh, M. B.; Oretskaya, T. S.; Shabarova, Z. A. Cyanogen Bromide-Induced Chemical Ligation: Mechanism and Optimization of the Reaction Conditions. Nucleosides and Nucleotides 1996, 15 (6), 1137-1147. DOI: 10.1080/07328319608007382.
(5) Nakamoto, K.; Abe, N.; Tsuji, G.; Kimura, Y.; Tomoike, F.; Shimizu, Y.; Abe, H. Chemically synthesized circular RNAs with phosphoramidate linkages enable rolling circle translation. Chemical Communications 2020, 56 (46), 6217-6220, 10.1039/DOCC02140G. DOI: 10.1039/DOCC02140G.
(6) Rigden, J. E.; Rezaian, M. A. In Vitro synthesis of an infectious viroid: Analysis of the infectivity of monomeric linear CEV. Virology 1992, 186 (1), 201-206. DOI: https://doi.org/10.1016/0042-6822 (92) 90074-Y.
(7) Chen, C.-y.; Sarnow, P. Initiation of Protein Synthesis by the Eukaryotic Translational Apparatus on Circular RNAs. Science 1995, 268 (5209), 415-417. DOI: 10.1126/science.7536344 (acccessed 2022 Oct. 7).
(8) Litke, J. L.; Jaffrey, S. R. Highly efficient expression of circular RNA aptamers in cells using autocatalytic transcripts. Nature Biotechnology 2019, 37 (6), 667-675. DOI: 10.1038/s41587-019-0090-6 (acccessed 2022-10-07T13:55:58).
(9) Kumar, A.; Palmer, N.; Miyasaki, K.; Finburgh, E.; Xiang, Y.; Portell, A.; Dailamy, A.; Suhardjo, A.; Chew, W. L.; Kwon, E. J.; et al. Extensive <i> in vitro</i> and <i> in vivo</i>protein translation via <i> in situ</i>circularized RNAs. Cold Spring Harbor Laboratory: 2022.
(10) Ford, E.; Ares, M. Synthesis of circular RNA in bacteria and yeast using RNA cyclase ribozymes derived from a group I intron of phage T4. Proceedings of the National Academy of Sciences 1994, 91 (8), 3117-3121. DOI: 10.1073/pnas.91.8.3117 (acccessed 2022-10-07T13:11:48).
(11) Wesselhoeft, R. A.; Kowalski, P. S.; Anderson, D. G. Engineering circular RNA for potent and stable translation in eukaryotic cells. Nature Communications 2018, 9 (1). DOI: 10.1038/s41467-018-05096-6 (acccessed 2022-10-07T13:35:12).
(12) Wesselhoeft, R. A.; Kowalski, P. S.; Parker-Hale, F. C.; Huang, Y.; Bisaria, N.; Anderson, D. G. RNA Circularization Diminishes Immunogenicity and Can Extend Translation Duration In Vivo. Molecular Cell 2019, 74 (3), 508-520.e504. DOI: 10.1016/j.molcel.2019.02.015 (acccessed 2022-10-07T13:26:51).
(13) Ni, L.; Yamada, T.; Murata, A.; Nakatani, K. Mismatch binding ligand upregulated back-splicing reaction producing circular RNA in a cellular model. Chemical Communications 2022, 58 (22), 3629-3632. DOI: 10.1039/dlcc06936e (acccessed 2022-10-07T13:11:41).
(14) Qu, L.; Yi, Z.; Shen, Y.; Lin, L.; Chen, F.; Xu, Y.; Wu, Z.; Tang, H.; Zhang, X.; Tian, F.; et al. Circular RNA vaccines against SARS-COV-2 and emerging variants. Cell 2022, 185 (10), 1728-1744.e1716. DOI: 10.1016/j.cell.2022.03.044 (acccessed 2022-10-07T13:26:16).
(15) Nichols, N. M.; Tabor, S.; McReynolds, L. A. RNA Ligases. Current Protocols in Molecular Biology 2008, 84 (1), 3.15.11-13.15.14, https://doi.org/10.1002/0471142727.mb0315s84. DOI: https://doi.org/10.1002/0471142727.mb0315s84 (acccessed 2022 Oct. 7).
(16) Kaufmann, G.; Klein, T.; Littauer, U. Z. T4 RNA ligase: Substrate chain length requirements. FEBS Letters 1974, 46 (1-2), 271-275. DOI: 10.1016/0014-5793 (74) 80385-6 (acccessed 2022-10-07T13:11:45).
(17) Puttaraju, M.; Been, M. Group I permuted intron-exon (PIE) sequences self-splice to produce circular exons. Nucleic Acids Research 1992, 20 (20), 5357-5364. DOI: 10.1093/nar/20.20.5357 (acccessed 2022-10-10T16:55:04).
(18) Ramanathan, A.; Robb, G. B.; Chan, S.-H. mRNA capping: biological functions and applications. Nucleic Acids Research 2016, 44 (16), 7511-7526. DOI: 10.1093/nar/gkw551 (acccessed Oct. 7, 2022). Topisirovic, I.; Svitkin Yuri, V.; Sonenberg, N.; Shatkin Aaron, J. Cap and cap-binding proteins in the control of gene expression. Wiley Interdisciplinary Reviews: RNA 2010, 2 (2), 277-298. DOI: 10.1002/wrna.52 (acccessed 2018 May 6). Mitchell, S. F.; Walker, S. E.; Algire, M. A.; Park, E.-H.; Hinnebusch, A. G.; Lorsch, J. R. The 5′-7-Methylguanosine Cap on Eukaryotic mRNAs Serves Both to Stimulate Canonical Translation Initiation and to Block an Alternative Pathway. Molecular Cell 2010, 39 (6), 950-962. DOI: 10.1016/j.molcel.2010.08.021 (acccessed 2022-10-07T14:25:08).
(19) Koch, A.; Aguilera, L.; Morisaki, T.; Munsky, B.; Stasevich, T. J. Quantifying the dynamics of IRES and cap translation with single-molecule resolution in live cells. Nature Structural & Molecular Biology 2020, 27 (12), 1095-1104. DOI: 10.1038/s41594-020-0504-7.
(20) Warminski, M.; Sikorski, P. J.; Kowalska, J.; Jemielity, J. Applications of Phosphate Modification and Labeling to Study (m) RNA Caps. Topics in Current Chemistry 2017, 375 (1), 16. DOI: 10.1007/s41061-017-0106-y.
(21) Sikorski, P. J.; Warminski, M.; Kubacka, D.; Ratajczak, T.; Nowis, D.; Kowalska, J.; Jemielity, J. The identity and methylation status of the first transcribed nucleotide in eukaryotic mRNA 5′ cap modulates protein expression in living cells. Nucleic acids research 2020, 48 (4), 1607-1626.
(22) Chen, C.-K.; Cheng, R.; Demeter, J.; Chen, J.; Weingarten-Gabbay, S.; Jiang, L.; Snyder, M. P.; Weissman, J. S.; Segal, E.; Jackson, P. K.; et al. Structured elements drive extensive circular RNA translation. Molecular Cell 2021, 81 (20), 4300-4318.e4313. DOI: 10.1016/j.molcel.2021.07.042 (acccessed 2022 Oct. 7). Yang, Y.; Fan, X.; Mao, M.; Song, X.; Wu, P.; Zhang, Y.; Jin, Y.; Yang, Y.; Chen, L.-L.; Wang, Y.; et al. Extensive translation of circular RNAs driven by N6-methyladenosine. Cell Research 2017, 27 (5), 626-641. DOI: 10.1038/cr.2017.31 (acccessed 2022-10-07T13:35:12). Fan, X.; Yang, Y.; Chen, C.; Wang, Z. Pervasive translation of circular RNAs driven by short IRES-like elements. Nature Communications 2022, 13 (1). DOI: 10.1038/s41467-022-31327-y (acccessed 2022-10-10T11:25:34).
(23) Lei, M.; Zheng, G.; Ning, Q.; Zheng, J.; Dong, D. Translation and functional roles of circular RNAs in human cancer. Molecular Cancer 2020, 19 (1). DOI: 10.1186/s12943-020-1135-7 (acccessed 2022-10-07T13:34:49). Prats, A.-C.; David, F.; Diallo, L. H.; Roussel, E.; Tatin, F.; Garmy-Susini, B.; Lacazette, E. Circular RNA, the Key for Translation. International Journal of Molecular Sciences 2020, 21 (22), 8591. DOI: 10.3390/ijms21228591 (acccessed 2022-10-07T13:30:21).
(24) Martinez-Salas, E.; Francisco-Velilla, R.; Fernandez-Chamorro, J.; Embarek, A. M. Insights into Structural and Mechanistic Features of Viral IRES Elements. Frontiers in Microbiology 2018, 8, Review. Marques, R.; Lacerda, R.; Romão, L. Internal Ribosome Entry Site (IRES)-Mediated Translation and Its Potential for Novel mRNA-Based Therapy Development. Biomedicines 2022, 10
(8), 1865. DOI: 10.3390/biomedicines10081865 (acccessed 2023-02-13T14:08:04). Chen, R.; Wang, S. K.; Belk, J. A.; Amaya, L.; Li, Z.; Cardenas, A.; Abe, B. T.; Chen, C.-K.; Wender, P. A.; Chang, H. Y. Engineering circular RNA for enhanced protein production. Nature Biotechnology 2022. DOI: 10.1038/s41587-022-01393-0 (acccessed 2022-10-07T11:58:17).
(25) Nowotny, M.; Gaidamakov, S. A.; Ghirlando, R.; Cerritelli, S. M.; Crouch, R. J.; Yang, W. Structure of human RNase H1 complexed with an RNA/DNA hybrid: insight into HIV reverse transcription. Molecular cell 2007, 28 (2), 264-276.
(26) Jacques, J.-P.; Susskind, M. M. Use of electrophoretic mobility to determine the secondary structure of a small antisense RNA. Nucleic Acids Research 1991, 19 (11), 2971-2977. DOI: 10.1093/nar/19.11.2971 (acccessed 2022-10-07T16:18:29). Cebrián, J.; Kadomatsu-Hermosa, M. J.; Castán, A.; Martínez, V.; Parra, C.; Fernández-Nestosa, M. J.; Schaerer, C.; Martínez-Robles, M.-L.; Hernández, P.; Krimer, D. B.; et al. Electrophoretic mobility of supercoiled, catenated and knotted DNA molecules. Nucleic Acids Research 2015, 43 (4), e24-e24. DOI: 10.1093/nar/gku1255 (acccessed Oct. 7, 2022).
(27) Abe, B. T.; Wesselhoeft, R. A.; Chen, R.; Anderson, D. G.; Chang, H. Y. Circular RNA migration in agarose gel electrophoresis. Molecular Cell 2022, 82 (9), 1768-1777.e1763. DOI: 10.1016/j.molcel.2022.03.008 (acccessed 2022-10-07T13:26:14).
(28) Haque, F. M.; Grayson, S. M. The synthesis, properties and potential applications of cyclic polymers. Nature Chemistry 2020, 12 (5), 433-444. DOI: 10.1038/s41557-020-0440-5 (acccessed 2022-10-07T13:56:08).
(29) Vicens, Q.; Kieft, J. S. Thoughts on how to think (and talk) about RNA structure. Proceedings of the National Academy of Sciences 2022, 119 (17). DOI: 10.1073/pnas.2112677119 (acccessed 2022-10-07T12:03:32).
(30) Kibbe, W. A. OligoCalc: an online oligonucleotide properties calculator. Nucleic Acids Research 2007, 35 (suppl_2), W43-W46. DOI: 10.1093/nar/gkm234 (acccessed Oct. 10, 2022).
(31) Mamot, A.; Sikorski, P. J.; Siekierska, A.; de Witte, P.; Kowalska, J.; Jemielity, J. Ethylenediamine derivatives efficiently react with oxidized RNA 3′ ends providing access to mono and dually labelled RNA probes for enzymatic assays and in vivo translation. Nucleic Acids Research 2022, 50 (1), e3-e3. DOI: 10.1093/nar/gkab867 (acccessed Feb. 17, 2022).

1. A circRNA analog according to formula 1 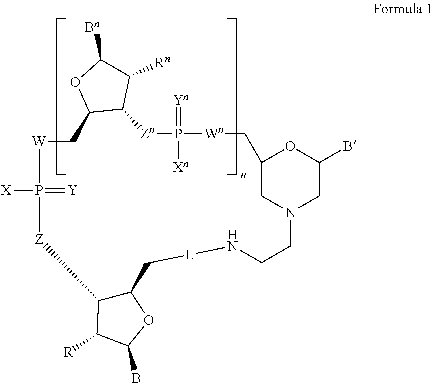
wherein:
n is any integer with a value from 1 to 20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker, wherein said linker L comprises a natural, modified or unnatural 7-methylguanosine cap structure.

2. A circRNA analog according to claim 1, wherein L is a structure according to Formula 2 or Formula 3: 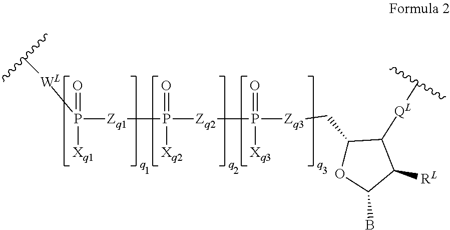 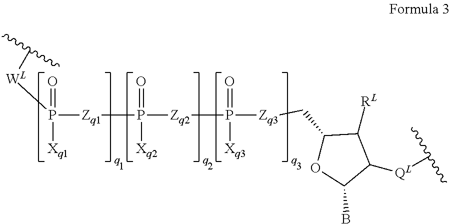
wherein:
B is 7-methylguanine, natural or unnatural 7-methylguanine derivative or analog,
each of q₁ through q₃ is independently 0, 1 or 2, wherein q₁+q₂+q₃ is equal 3 or 4;
X_{q1}, X_{q2}, X_{q3} is independently O, BH₃, or S;
Z_{q1} and Z_{q2} is independently O, NH, CH₂, C (halogen)₂ or CH (halogen);
Z_{q3} is O, S, NH, CH₂, C (halogen)₂ or CH (halogen);
W^{L} is a single chemical bond, O, NH, S, CH₂ or a linear or branched linker;
Q′ is a single chemical bond, CH₂, or a linear or branched linker;
R^{L} is H, OH, O-Me, O-alkyl, O-[(substituted)alkyl].

3. A circRNA analog according to claim 1, wherein L is a structure according to Formula 4 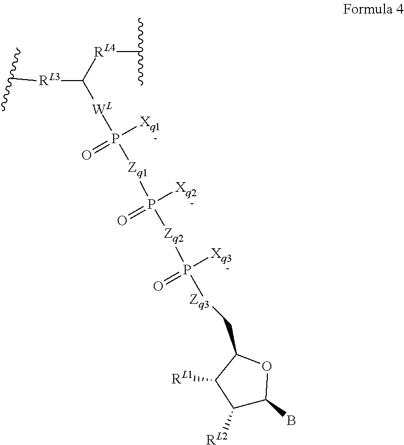
wherein:
B is 7-methylguanine, natural or unnatural 7-methylguanine derivative or analog,
each of q₁ through q₃ is independently 0, 1 or 2, wherein q₁+q₂+q₃ is equal 3 or 4
X_{q1}, X_{q2}, X_{q3} is independently O, BH₃, or S;
Z_{q1} and Z_{q2} is independently O, NH, CH₂, C (halogen)₂ or CH (halogen);
Z_{q3} is O, S, NH, CH₂, C (halogen)₂ or CH (halogen);
W^{L} is a single chemical bond, O, NH, S, CH₂ or a linear or branched linker;
R^{L1} and R^{L2} are independently H, OH, O-Me, O-alkyl, O-[(substituted)alkyl], wherein at least one of R^{L1} and R^{L2} is not OH;
R^{L3} and R¹⁴ are independently single chemical bonds, CH₂ or a linear or branched linker.

4. A circ analog according to claim 2, wherein L is a structure selected among of following formulas:

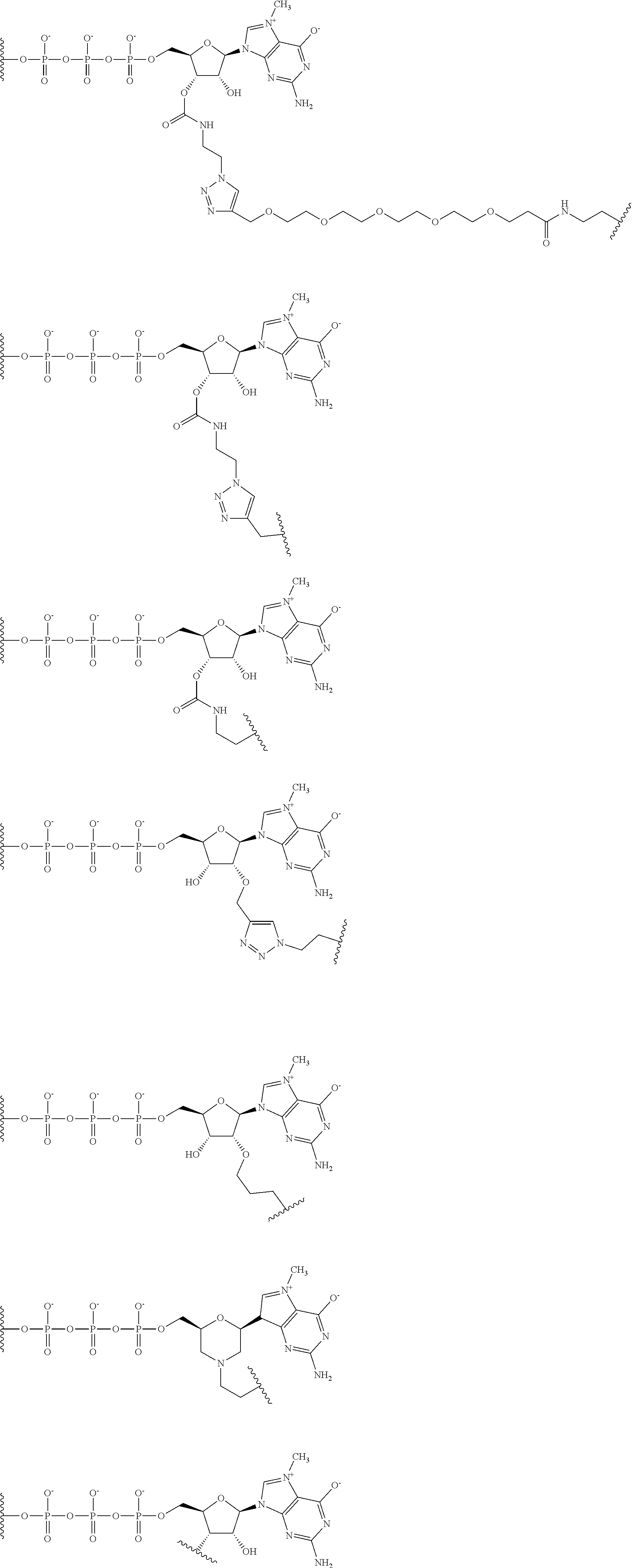

5. A circ analog according to claim 3, wherein L is a structure selected among of following formulas:

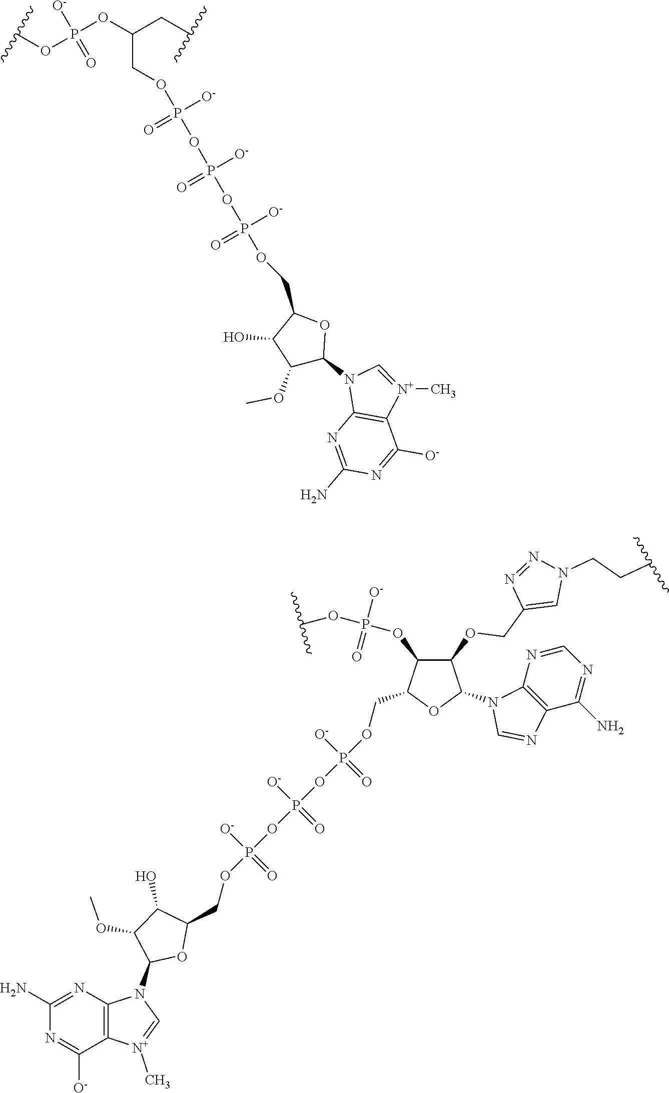 [SMILES: *CC(COP(=O)([O-])OP(=O)([O-])OP(=O)([O-])OC[C@H]1O[C@@H](n2c[n+](C)c3c([O-])nc(N)nc32)[C@H](OC)[C@@H]1O)OP(=O)([O-])O* ; *CCn1cc(CO[C@@H]2[C@H](OP(=O)([O-])O*)[C@@H](COP(=O)([O-])OP(=O)([O-])OP(=O)([O-])OC[C@H]3O[C@@H](n4c[n+](C)c5c([O-])nc(N)nc54)[C@H](OC)[C@@H]3O)O[C@H]2n2cnc3c(N)ncnc32)nn1]

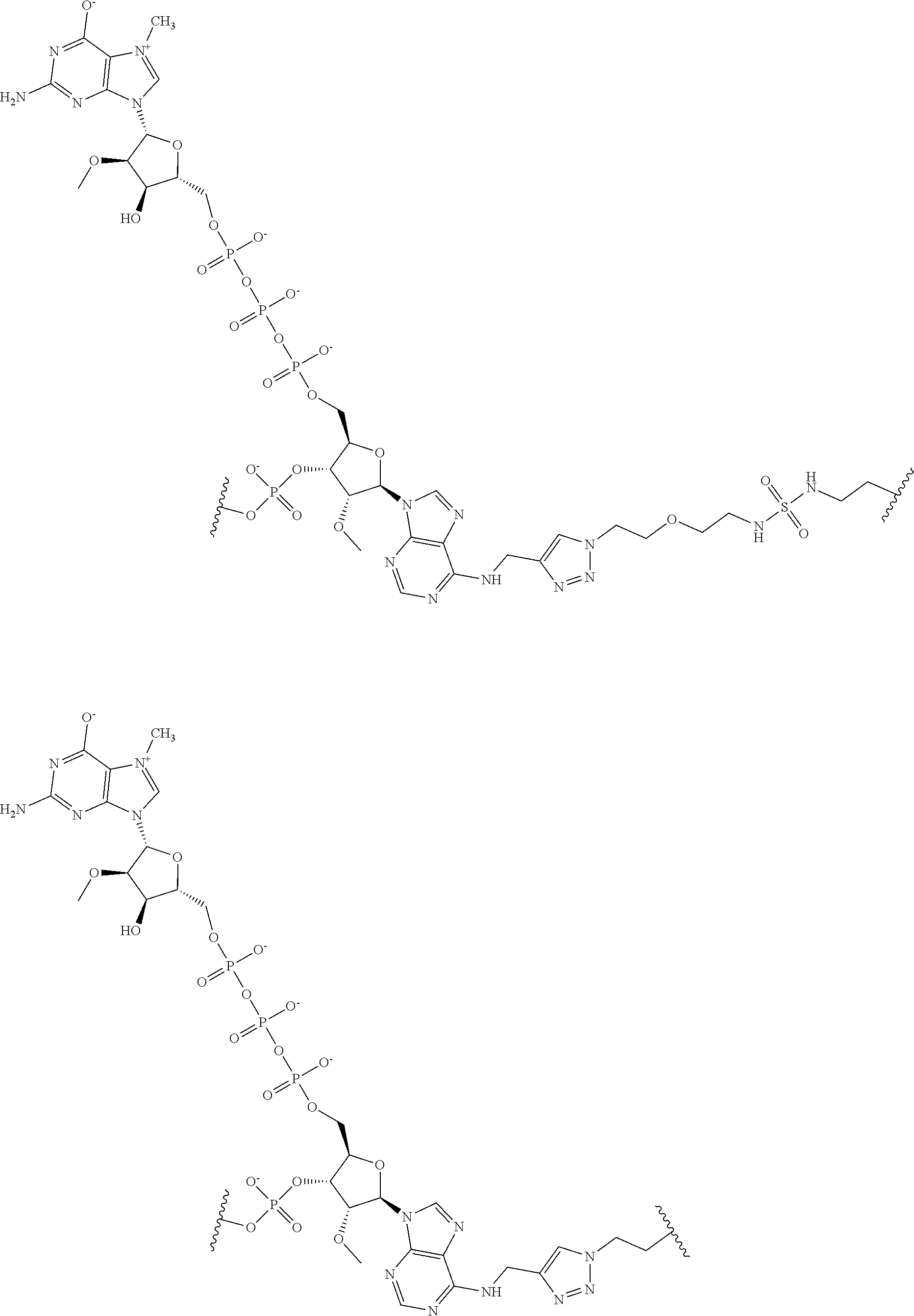 [SMILES: *CCNS(=O)(=O)NCCOCCn1cc(CNc2ncnc3c2ncn3[C@@H]2O[C@H](COP(=O)([O-])OP(=O)([O-])OP(=O)([O-])OC[C@H]3O[C@@H](n4c[n+](C)c5c([O-])nc(N)nc54)[C@H](OC)[C@@H]3O)[C@@H](OP(=O)([O-])O*)[C@H]2OC)nn1 ; *CCn1cc(CNc2ncnc3c2ncn3[C@@H]2O[C@H](COP(=O)([O-])OP(=O)([O-])OP(=O)([O-])OC[C@H]3O[C@@H](n4c[n+](C)c5c([O-])nc(N)nc54)[C@H](OC)[C@@H]3O)[C@@H](OP(=O)([O-])O*)[C@H]2OC)nn1]

6. A circRNA analog according to claim 1, wherein the linker L comprises additionally a detectable label.

7. A method of synthesis of circRNA analogs defined in claims 1-6, characterized by that a circRNA analog precursor (formula 8) 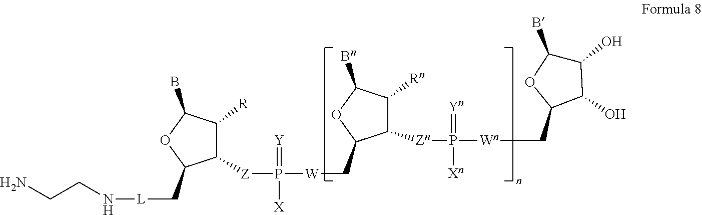
wherein:
n=1-20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker, wherein said linker L comprises a natural, modified or unnatural 7-methylguanosine cap structure;
is first oxidized during step (i) of the method, yielding a dialdehyde derivative (formula 9), 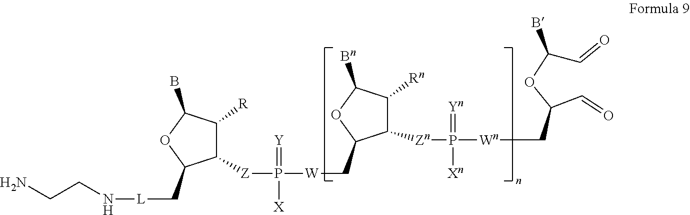
wherein:
n=1-20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker, wherein said linker L comprises a natural, modified or unnatural 7-methylguanosine cap structure;
which is next incubated in reductive environment during step (ii) of the method, yielding the desired circRNA analog.

8. A method, according to claim 7, characterized by that step (i) and step (ii) are carried out in one reactor.

9. A method, according to claim 7, characterized by that step (i) of the method is carried out in the presence of NaIO₄, preferably at a concentration below 10 mM, at a temperature below 60° C.

10. A method, according to claim 7, characterized by that step (ii) of the method is carried out in buffered solution, preferably at pH 5-8, in presence of NaBH₃CN below 100 mM.

11. A method, according to claim 7, characterized by that step (i) or step (ii) of the method are carried out in presence of a natural, modified or unnatural nucleic acid oligomer that is complementary to a RNA precursor sequence.

12. A method, according to claim 7, characterized by that the circRNA analog is isolated from the reaction mixture by a known method of RNA isolation, such as precipitation in alcohol, size exclusion chromatography, gel electrophoresis, or high-performance liquid chromatography (HPLC).

13. Use of a circRNA analog according to claims 1-6 in protein production in an in vitro setup, which includes but is not limited to artificial reaction mixture, cellular lysates, nuclear lysates, cell cultures, and tissue cultures.

14. A circRNA analog according to claims 1-6 for use in therapeutic or diagnostic method, in particular for protein production in an in vivo setup, which includes but is not limited to whole organisms, organs, cellular and tissue transplants.

15. A pharmaceutical composition comprising of a circRNA analog according to claims 1-6.
